(19) United States
(12) Patent Application Publication
(10) Pub. No.: US 2025/0010092 A1
(43) Pub. Date: Jan. 9, 2025

(54) DEVICES AND METHODS FOR REPAIR AND RAPID HEALING OF INTERNAL BODY STRUCTURES USING PHOTOBIOMODULATION THERAPY

(71) Applicant: Prometheus Therapeutics Inc., Miramar, FL (US)

(72) Inventors: Baruch B. LIEBER, Aventura, FL (US); Ajay K. WAKHLOO, Weston, MA (US); Timothy Michael MCSWEENEY, South Miami, FL (US)

(73) Assignee: Prometheus Therapeutics Inc., Miramar, FL (US)

(21) Appl. No.: 18/894,943

(22) Filed: Sep. 24, 2024

Related U.S. Application Data
(63) Continuation of application No. PCT/US2023/074511, filed on Sep. 18, 2023.
(60) Provisional application No. 63/483,962, filed on Feb. 8, 2023.
(60) Provisional application No. 63/407,546, filed on Sep. 16, 2022.

(51) Int. Cl. A61N 5/06

(52) U.S. Cl. CPC A61N 5/0603; A61N 2005/063; A61N 2005/066; A61N 2005/0663

(57) ABSTRACT

In some embodiments, a distal end of a catheter can be disposed within a body cavity (e.g., a CSF-filled space) of a subject near a target region of tissue (e.g., brain tissue) to be treated. The catheter can include a catheter body and a light emitter configured to emit light asymmetrically and at a non-zero angle relative to a central axis of the catheter body. Light can be emitted from the light emitter asymmetrically and a non-zero angle relative to a central axis of the catheter body onto the target region of tissue.

### CROSS-REFERENCE TO RELATED APPLICATIONS

This application is a continuation of International Application No. PCT/US2023/074511, entitled “Devices and Methods for Repair and Rapid Healing of Internal Body Structures Using Photobiomodulation Therapy,” filed Sep. 18, 2023, which claims priority to and the benefit of U.S. Provisional Patent Application No. 63/407,546, entitled “Devices and Methods for Repair and Rapid Healing of Internal Body Structures Using Photobiomodulation Therapy,” filed Sep. 16, 2022, and U.S. Provisional Patent Application No. 63/483,962, entitled “Devices and Methods for Repair and Rapid Healing of Internal Body Structures Using Photobiomodulation Therapy,” filed Feb. 8, 2023, the disclosures of each of which are incorporated herein by reference in their entireties.

This application is related to U.S. patent application Ser. No. 15/976,199, now U.S. Pat. No. 11,191,976, entitled “Devices and Methods for Repair of a Selected Blood Vessel or Part Thereof and Rapid Healing of Injured Internal Body Cavity Walls,” filed May 10, 2018, which claims priority to and the benefit of U.S. Provisional Patent Application Ser. No. 62/508,690, entitled “Devices and Methods for Repair of a Selected Blood Vessel or Part Thereof and Rapid Healing of Injured Internal Body Cavity Walls,” filed May 19, 2017, the disclosures of each of which are incorporated herein by reference in their entireties.

### TECHNICAL FIELD

The present disclosure relates generally to systems, apparatus, and methods for therapeutic intervention in blood vessels and other body lumens or cavities.

### BACKGROUND

The embodiments described herein relate generally to imparting therapeutic light energy to cellular elements to invoke a healing response, such as modulating blood flow (e.g., reducing flow rate or stopping flow rate completely) to a selected blood vessel or part thereof.

The delivery of photobiomodulation (PBM) therapy includes the application of low level energy of coherent or non-coherent (e.g., LED) light selected from a range of wavelengths to target tissue. The light energy can optionally be applied in conjunction with a photosynthesizer (e.g., introduced intravenously or locally), such as sodium fluorescein, erythrosine B, or rose bengal dye, to aid in the absorption of the light energy into the target tissue. For example, a photosensitizer can be absorbed onto an endothelial luminal surface of an injured artery and light energy can be applied to the endothelial luminal surface to invoke a healing response.

A mechanistic explanation of the healing response to the incident light is that light sensitive cellular chromophores such as cytochrome c oxidase (unit IV in the mitochondrial respiratory chain) absorb the incident photons that dissociate inhibitory nitric oxide from the enzyme, leading to an increase in electron transport, mitochondrial membrane potential and adenosine triphosphate (ATP) production. Another hypothesis suggests that light-sensitive ion channels can be activated allowing calcium to enter the cell. After the initial photon absorption events, numerous signaling pathways are activated via reactive oxygen species, cyclic adenosine monophosphate (AMP), nitric oxide (NO), and calcium ions (Ca2+), leading to activation of transcription factors. These transcription factors can lead to increased expression of genes related to protein synthesis, cell migration and proliferation, anti-inflammatory signaling, anti-apoptotic proteins, and antioxidant enzymes. Stem cells and progenitor cells appear to be particularly susceptible to PBM.

Known approaches to the treatment of neurogenerative disorders using PBM rely primarily on noninvasive transcutaneous and trans-bone transmission of light energy. Thus, the transmission of light energy to target issue is limited to a range of wavelengths that can penetrate the soft and hard tissue. Due to attenuation, a much larger energy density (i.e., energy per unit area) than is needed at the target tissue is typically applied. Additionally, energy density of the light onto the target tissue typically cannot be well controlled to be relatively uniform (i.e., to be maintained within a range of energy density values that is high enough to be therapeutically effective and low enough not to damage the target tissue).

Additionally, patients having a chronic subdural hematoma (cSDH) present with a variety of symptoms of different severity. They can range from headaches, isolated seizure, cognitive decline, stroke-like symptoms like numbness, facial asymmetry, to aphasia, weakness, paralysis, altered mental status, and death. Current research suggests that cSDH may result from an initial injury to the dural border cell layer on the inner surface of the dura, a thick membrane that covers the brain, which tears and leads to the extravasation of cerebrospinal fluid and blood in the space between the broken cell layer and the rest of the dura. The initial injury to the dural border cell layer in some patients is unable to be repaired and thus stimulates a cycle of hyperfibrinolysis, inflammation, angiogenesis, and the resultant development of subdural neo-membranes.

For the patients with moderate or severe symptoms, surgical drainage, either through burr-holes or a craniotomy, is the typical standard of care. Surgical drainage improves neurologic status via decompression of the brain matter and prevents further progression in patients with large cSDH. Additionally, minimally invasive clinical trials are assessing the benefit of middle meningeal artery (MMA) embolization. In this approach, a microcatheter is placed into an MMA or its branches and embolic agents such as polyvinyl alcohol particles, detachable coils, liquid embolics, e.g., Onyx, Squid, Phil, and cyano-acrylates are infused or injected into the MMA to stop the blood supply to the inflamed dura and thus halt the inflammatory cycle. Embolization either is accomplished alone for treatment of a cSDH or as an adjunct to surgical decompression.

A significant problem with surgical drainage, however, is recurrence. Previous studies report a recurrence rate of approximately 10-30% and a mortality rate of 4% after surgical drainage. Complications of middle meningeal artery embolization by minimally invasive microcatheterization related to inadvertent occlusion of important branches include stroke, hemorrhage, blindness and facial paralysis. Other problems are associated with MMA embolization, such as recurrence of cSDH and a lack of clinical improvement.

Thus, there is a need for systems, apparatus, and methods capable of more effectively treating target tissue with PBM, particularly with respect to treating diseases and repairing injuries that impact degenerative disorders of the brain and the spinal cord as well as target tissue included in or accessible via large synovial joints.

### SUMMARY

In some embodiments, a distal end of a catheter can be disposed within a body cavity (e.g., a CSF-filled space) of a subject near a target region of tissue (e.g., brain tissue) to be treated. The catheter can include a catheter body and a light emitter configured to emit light asymmetrically and at a non-zero angle relative to a central axis of the catheter body. Light can be emitted from the light emitter asymmetrically and a non-zero angle relative to a central axis of the catheter body onto the target region of tissue.

### BRIEF DESCRIPTION OF THE DRAWINGS

FIG. 1 is a schematic block diagram of a system, according to an embodiment.

FIGS. 2A-2C are schematic illustrations of a light scatterer of the system of FIG. 1.

FIGS. 3A-3D are schematic illustrations of a fluid conduit and inner body for a light conduit of the system of FIG. 1.

FIGS. 4A-4G are schematic illustrations of a spacing member of the system of FIG. 1.

FIGS. 5A-5B are schematic illustrations of an imager of the system of FIG. 1.

FIG. 6 is a schematic illustration of a mesh tube usable with the system of FIG. 1.

FIGS. 7A and 7B are schematic illustrations of occlusion devices usable with the system of FIG. 1.

FIG. 8 is a schematic illustration of an introducer usable with the system of FIG. 1.

FIG. 9 a flowchart illustrating a method, according to an embodiment.

FIG. 10 is a schematic illustration of a kit including elements of a system for use in a method according to an embodiment.

FIGS. 11A-11E are schematic illustrations of devices and methods for treating an aneurysm in a blood vessel, according to embodiments.

FIGS. 12A-12E are schematic illustrations of devices and methods for treating an aneurysm at a bifurcation in a blood vessel, according to embodiments.

FIG. 13 is a schematic illustration of a device and method for treating a fusiform aneurysm in a blood vessel, according to an embodiment.

FIG. 14 is a schematic illustration of a device and method for occluding a blood vessel, according to an embodiment.

FIG. 15 is a schematic illustration of a device and method for embolizing a malformation in a blood vessel, according to an embodiment.

FIG. 16 is a schematic illustration of a device and method for treating a cavernous malformation, according to an embodiment.

FIG. 17A is a schematic illustration of a device and method for treating a joint, e.g., a knee capsule.

FIG. 17B is an enlarged side view of the device of FIG. 17A, and FIG. 17C is an end view of the device of FIG. 17B.

FIGS. 17D and 17E are side and end views, respectively, of an alternative embodiment of a device suitable for use in the method for treating a joint as shown in FIG. 17A.

FIGS. 17F and 17G are side and end views, respectively, of yet another alternative embodiment of a device suitable for use in the method for treating a joint as shown in FIG. 17A.

FIG. 17H is an end view of an alternative embodiment of a device suitable for use in the method for treating a joint as shown in FIG. 17A.

FIGS. 18A-20 are various schematic illustrations of portions of a human brain, spinal cord, associated CSF spaces, and other related body portions.

FIG. 21 is a schematic block diagram of a system, according to an embodiment.

FIG. 22A is a schematic illustration of a cross-section of a portion of a system, according to an embodiment.

FIG. 22B is a schematic illustration of a cross-section of a portion of the system of FIG. 22A including a light scatterer, a reflective surface portion, and a radiopaque marker having two projecting portions.

FIG. 22C is a schematic illustration a radiopaque marker of the system of FIG. 22A.

FIG. 22D is a schematic illustration a radiopaque marker of the system of FIG. 22B.

FIG. 22E is a schematic illustration of a cross-section of a portion of the system of FIG. 22A including imaging conduits and a reflective surface portion.

FIG. 22F is a schematic illustration of cross-section of a portion of the system of FIG. 22A including an end cap and the radiopaque marker shown in FIG. 22D.

FIG. 23A is a schematic illustration of a cross-section of a portion of a system, according to an embodiment.

FIG. 23B is a schematic illustration of a cross-section of a portion of a system, according to an embodiment.

FIGS. 24A-24G are schematic illustrations of cross-sections of various spacing members, according to an embodiment.

FIGS. 24H and 241 are schematic illustrations of a perspective view and a cross-sectional view of a spacing members, according to an embodiment.

FIG. 25 is a schematic illustration of cross-sections of various expanded configurations of spacing members, according to an embodiment

FIGS. 26A and 26B are schematic illustrations of a cross-section of a portion of a system in a first configuration and a second configuration, respectively, according to an embodiment.

FIG. 27 is a schematic illustration of a cross-section of a portion of a system, according to an embodiment

FIG. 28 is a schematic illustration of a cross-section of a portion of a system, according to an embodiment

FIG. 29 is a schematic illustration of a cross-section of a portion of a system, according to an embodiment.

FIG. 30 is a schematic illustration of a cross-section of a portion of a system, according to an embodiment.

FIG. 31 is a schematic illustration of a cross-section of a portion of a system, according to an embodiment.

FIG. 32 is a schematic illustration of a cross-section of a portion of a system, according to an embodiment.

FIG. 33 is a flow chart illustrating a method, according to an embodiment.

FIG. 34 is a schematic illustration of a device and method for treating a stroke region of a brain, according to an embodiment.

### DETAILED DESCRIPTION

Systems and methods are disclosed that are suitable for effectively treating blood vessels, including cerebral, coronary, and peripheral vessels, for aneurysms or other malformations (such as arteriovenous and dural malformations), as well as for blood vessel occlusion to devascularize tumors and to treat varicose and spider veins to exclude them from the circulation. The systems and methods may also be used for treating ulcerations in internal body cavity walls such as stomach ulcers, parenchymal tumors, such as brain tumors, liver tumors and other soft tissue non-vascular lesions of the body, bleeding vascular structures, arterial or venous (not by endovascular access), hemorrhoidal bleeding, esophageal varices, spider hemangiomas, bleeding of the joints, amyloid generative diseases, lymphangiomas, cartilage damage, joint inflammation such as rheumatoid arthritis, synovial joint inflammation and traumatic joint injury, bone repair, kidney tumor and inflammation disorders such as fibrosis, lungs Broncho-pulmonary system bleeding, myocardial injury, carotid disease, neurodegenerative disorders, and splenomegaly.

The disclosed system includes a catheter device that may include a catheter body with a distal end and a proximal end and a light emitter disposed at the distal end of the catheter body and configured to emit light. A fluid conduit is disposed in the catheter body, extending from the proximal end to the distal end of the catheter body. The fluid conduit has an inlet at the proximal end of the catheter body and is coupleable to a source of fluid, and has an outlet at the distal end and is configured to discharge fluid from the source via the conduit and out of the distal end. A spacing member is disposed at the distal end of the catheter body and is reconfigurable from a collapsed configuration to an expanded configuration. In the expanded configuration, the spacing member is disposed about the light emitter to maintain the light emitter approximately centered within the spacing member with respect to at least one axis of the spacing member. The spacing member is at least partially transmissive and/or transflective of the light emitted from the light emitter. The apparatus is configured for the distal end of the catheter body to be inserted at least partially into a body lumen having an interior wall, for the spacing member to be disposed in the expanded configuration within the body lumen, for fluid to be discharged into the body lumen, and for light to be emitted from the light emitter to illuminate the interior wall of the body lumen.

In some embodiments, a method includes disposing into a blood vessel of a subject adjacent to a region of a wall of the blood vessel to be treated, a distal end of a catheter. The distal end of the catheter is disposed at the center or proximal end of a treatment region. The catheter has disposed at the distal end thereof: a light emitter configured to emit light; an outlet of a fluid conduit coupled to a source of fluid; and a spacing member reconfigurable from a collapsed configuration to an expanded configuration. The spacing member can be at least partially transmissive and/or transflective of the light emitted from the light emitter, and porous to fluid discharged from the fluid outlet. The spacing member can be moved to the expanded configuration when disposed at the treatment location. When in the expanded configuration, the spacing member can be disposed about the light emitter to maintain the light emitter approximately centered within the spacing member with respect to at least one axis of the spacing member. The method further includes disposing the spacing member approximately centered within the blood vessel lumen. A fluid is discharged from the outlet of the fluid conduit into the blood vessel to dilute the blood in the blood vessel with the fluid. Light is emitted from the light emitter through the diluted blood in the blood vessel lumen and onto the region of the wall of the blood vessel to be treated.

In some embodiments, a distal end of a catheter can be disposed within a body cavity (e.g., a CSF-filled space) of a subject near a target region of tissue (e.g., brain tissue) to be treated. The catheter can include a catheter body and a light emitter configured to emit light asymmetrically and at a non-zero angle relative to a central axis of the catheter body. Light can be emitted from the light emitter asymmetrically and a non-zero angle relative to a central axis of the catheter body onto the target region of tissue.

In some embodiments, a system, includes: a catheter body; a light conduit at least partially disposed within the catheter body; a light emitter disposed at a distal end of the light conduit and, when disposed in a body cavity of a patient near a target tissue region, configured to emit a first portion of light received through the light conduit asymmetrically and at a non-zero angle relative to a central axis of the light conduit such that the first portion is transmitted to the target tissue region at a first intensity and to emit a second portion of the light received through the light conduit distally of the light emitter; and a light scatterer coupled to the light emitter and configured to diffuse the second portion of light such that the second portion of light is transmitted to a non-target tissue region at second intensity lower than the first intensity.

In some embodiments, a system includes a catheter body, a light conduit, and a light emitter. The light conduit can be at least partially disposed within the catheter body. The light emitter can be disposed at the distal end of the light conduit and configured to emit light asymmetrically and at a non-zero angle relative to a central axis of the light conduit such that the light is transmitted to a target region of tissue when the light emitter is disposed in a body cavity of a patient near (e.g., adjacent) to the target tissue region.

In some embodiments, a system includes a catheter body defining a working channel, an inner body translatable within the working channel and including a light emitter on a distal end of the inner body, and a spacing member. The spacing member can be coupled to the inner body proximal of the light emitter and configured to transition between a collapsed configuration and an expanded configuration. The spacing member can have a conical shape in the expanded configuration such that spacing member defines a conical space within which the light emitter is at least partially disposed, and the spacing member can be configured to maintain the light emitter approximately centered with respect to a central axis of the spacing member when the spacing member is in the expanded configuration within a body lumen or between opposing walls of a body cavity.

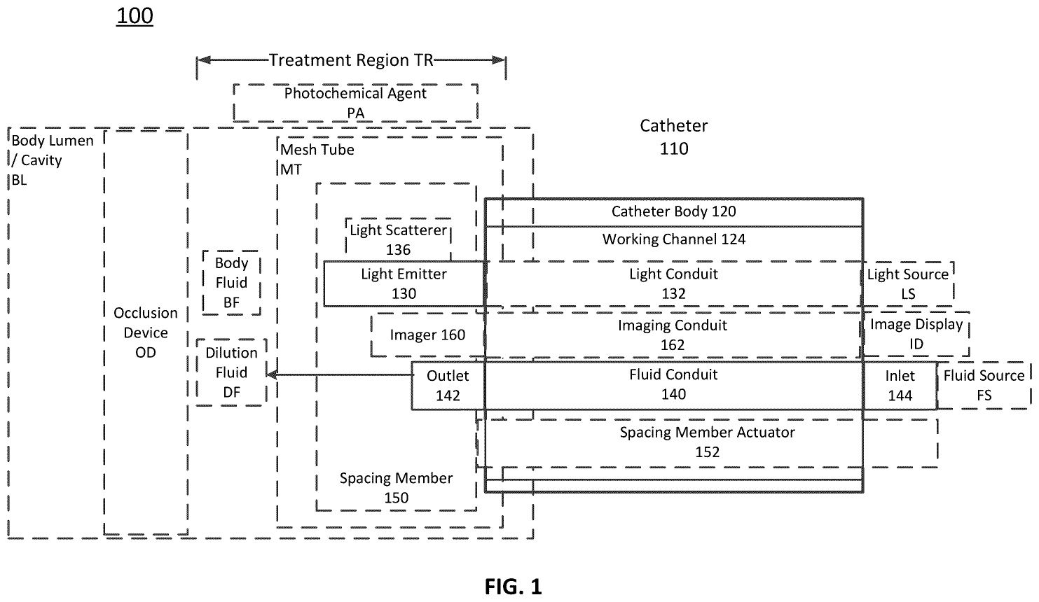

As illustrated schematically in FIG. 1, a treatment system 100 can include a catheter 110, which may be operatively coupled to other devices or systems, including a light source LS, a fluid source FS, and an image display ID, and may be used in conjunction with other devices, including a mesh tube MT, an occlusion device OD, and an introducer (not shown in FIG. 1), and with compositions such as a photochemical agent PA.

Catheter 110 may have an elongate catheter body 120 with a proximal end and a distal end suitable for insertion into a body lumen or cavity BL, such as a blood vessel, adjacent to a treatment region TR of the body lumen or cavity BL. Catheter body 120 may define an internal working channel 124, in which other components of catheter 110 can be disposed, and may be moveable therethrough. Thus, in some embodiments catheter body 120 may be inserted into the body of the patient, such as by delivery over a guidewire through the vasculature of the patient, until the distal end is disposed adjacent to the treatment region TR. The guidewire can then be removed and the other components of catheter 110 can be delivered through working channel 124 until their distal ends are disposed at the treatment region TR in appropriate working relation to the distal end of catheter body 120. In other embodiments, some or all of the other components of catheter 110 may be disposed in and/or coupled to catheter body 120 before catheter 110 is inserted into the body of the patient and the distal end delivered to the treatment region TR.

Catheter 110 includes a light emitter 130, which is disposed at the distal end of catheter body 120 when catheter 110 is configured for use. Light emitter 130 may be optically coupled to the light source LS by a light conduit 132, which may be disposed within catheter body 120, e.g. in working channel 124, and extends from the proximal to the distal end of the catheter body 120.

Catheter 110 also includes a fluid conduit 140, which may be disposed within catheter body 120 and extending from an inlet 144 at the proximal end of catheter body 120 to an outlet 142 at the distal end of catheter body 120. Fluid conduit 140 may be coupled at inlet 144 to the fluid source FS.

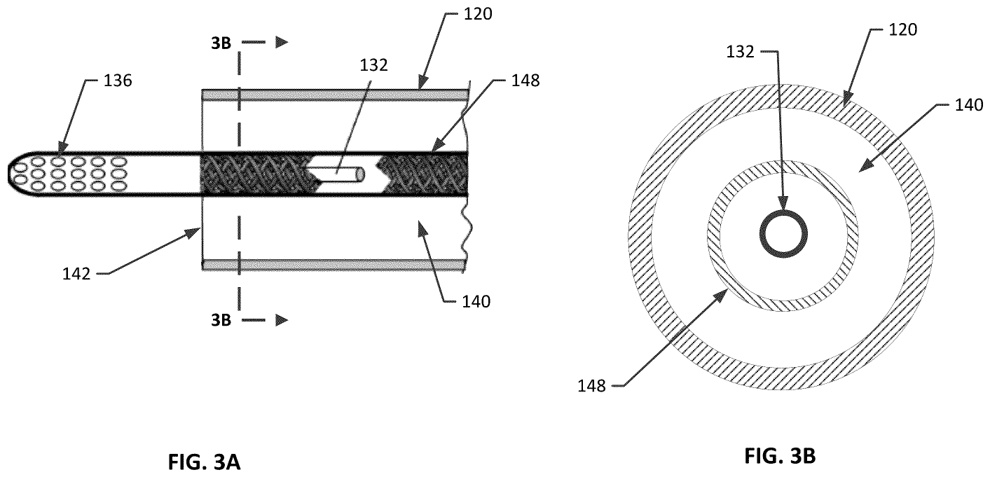

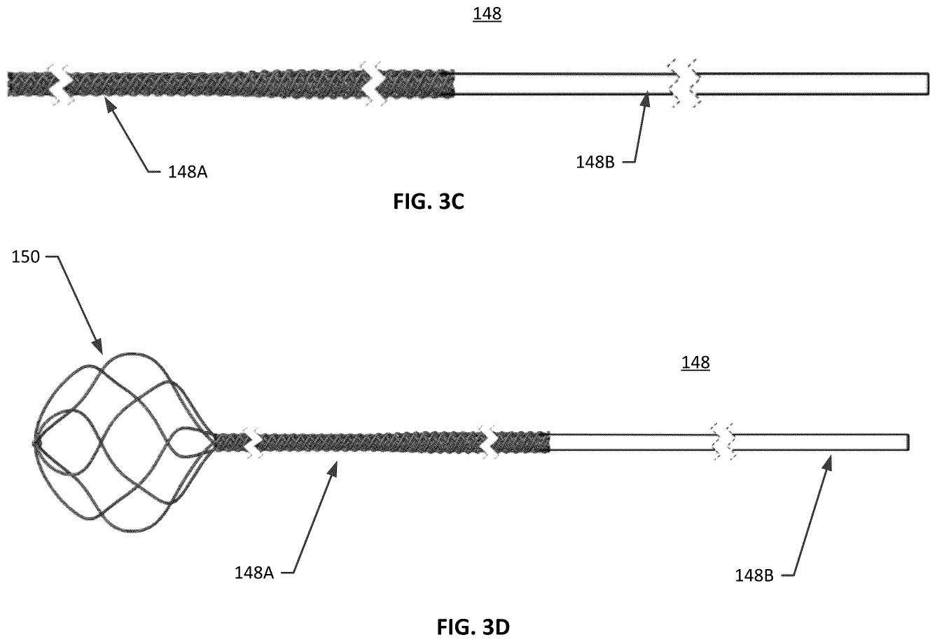

Catheter 110 may also include a spacing member 150 disposed at the distal end of catheter body 120. Depending on the implementation of spacing member 150, it may be attached to the distal end of catheter body 120 and actuated by fluid through fluid conduit 140. In some embodiments, spacing member 150 is coupled to or integrally formed with another component of the catheter 110. For example, the spacing member 150 can be formed with or coupled to inner body 148 (as described for example, with respect to FIG. 3D) that can now function as a spacing member actuator 152 that is disposed within catheter body 120. The spacing member actuator 152 extends from the proximal end to the distal end of the catheter body 120, and can be used to move the spacing member 150 between a collapsed configuration and an expanded configuration.

Catheter 110 may also include an imager 160 coupled to the distal end of catheter body 120. Imager 160 may be optically coupled to the image display ID by an imaging conduit 162 that may be disposed within catheter body 120 and extend from the proximal end to the distal end of catheter body 120.

Each component of treatment system 100 can be implemented in various ways. For applications in which catheter 110 is to be used to access treatment region TR of body lumen BL endovascularly, catheter 110 can be implemented as a conventional endovascular catheter, including its construction and materials, the ability to steer or not steer or deflect the distal end, to be deliverable over a guide wire or not, and include user controls and fittings at the proximal end. In some embodiments, the guide wire (not shown) can be disposed in working channel 124 of catheter body 120. In other embodiments, e.g., in which catheter body 120 may be relatively large, catheter body 120 may include a dedicated guide wire lumen, separate from working channel 124. The proximal portion of the catheter body 120 can be stiffer than the distal portion to provide sufficient rigidity for a user to push catheter body 120 over the guide wire and through the lumen, e.g., vasculature. The more flexible distal portion can facilitate navigation of catheter body 120 through, for example, tortuous vasculature. Catheter body 120 may be introduced into the body lumen BL, such as a blood vessel, via a cut down or other percutaneous technique for accessing the vessel lumen. In some applications, catheter 110 may be used to access treatment region TR directly, rather than through the subject's vasculature, and may be implemented accordingly. For example, if catheter 110 is used to access a treatment region TR directly through soft tissue, it may be implemented as a relatively rigid needle inserted through a trocar.

Light emitter 130 may be implemented with any known, suitable construction for emitting light of the desired wavelength and intensity from the distal end of catheter 110 to the treatment region TR of the body lumen BL. In some embodiments, light emitter 130 may simply be the end of an optical fiber, and the optical fiber may function as light conduit 132 to convey light from the light source LS coupleable to the proximal end of catheter 110. Light source LS may be any suitable source of light of the desired wavelength and intensity, and may be a source of coherent light such as a laser (pulsed or continuous wave), or incoherent light (such as a xenon or halogen light and a suitable bandpass filter). In other embodiments, light emitter 130 may be a relatively compact light source, e.g., a light emitting diode (LED) or a laser diode, disposed at the distal end of catheter 110, to which electrical power is provided by a conductor extending from the proximal end of catheter 110 through catheter body 120 to the light source. In alternative embodiments, a LED or a laser diode can be disposed at the proximal end of catheter 110.

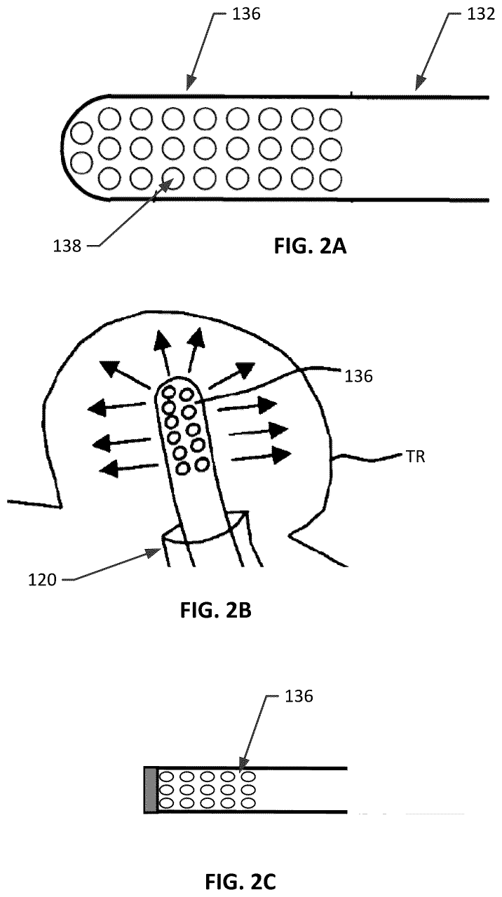

To produce a desired distribution of light at the treatment region TR, i.e., a distribution different from that produced by light source LS, in some embodiments a light scatterer 136 (see, e.g., FIG. 2A) is operatively associated with light source LS to scatter light from the light source LS across treatment region TR. In some embodiments, for example, as shown schematically in FIG. 2A, light scatterer 136 may be implemented as a convex end cap on the distal tip of light conduit 132, e.g., an optical fiber. The end cap may include light scattering particles, illustrated in FIG. 2A as circular regions 138. Such particles may be, for example, titanium dioxide. Other light scattering materials (high refractive index of ˜2.5) or refracting structures may be used, such as, for example, diffraction gratings.

As shown schematically in FIG. 2B, the light emitter 130 is extended out a distal end of the catheter body 120, and the end cap and light scattering particles diffuse and distribute the light emitted from the distal tip of optical fiber 132 around the lateral sides of the tip and end cap to apply light to the treatment region TR, which in this instance is the surface of an aneurysm. The distribution pattern for the light may be tailored to the shape of the treatment region TR, with the objective of producing a relatively uniform energy density at the surface of treatment region TR, avoiding regions of excessively high energy density, i.e., “hot spots.” For example, in the embodiment shown in FIG. 2B, the distribution is approximately spherical, to correlate with the approximately spherical shape of the vascular aneurysm that is the treatment region TR. In other embodiments, such as that shown in FIG. 2C, rather than a convex end cap, light scatterer 136 may be implemented as a cylindrical tip that scatters light only radially and not axially, thus producing a light distribution that better correlates to a cylindrical treatment region TR, such as the wall of a body lumen such as a blood vessel.

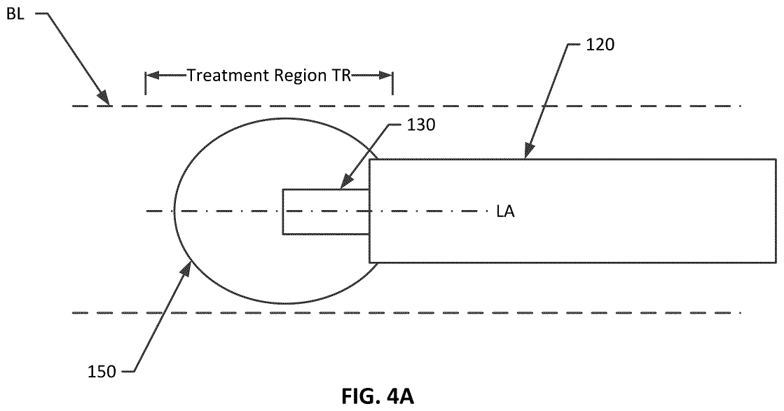

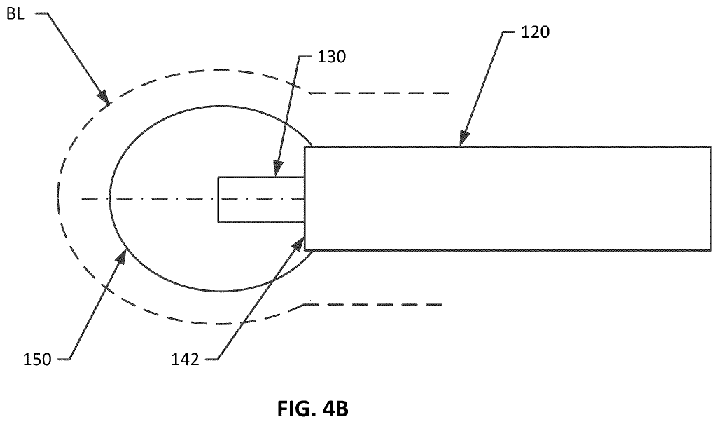

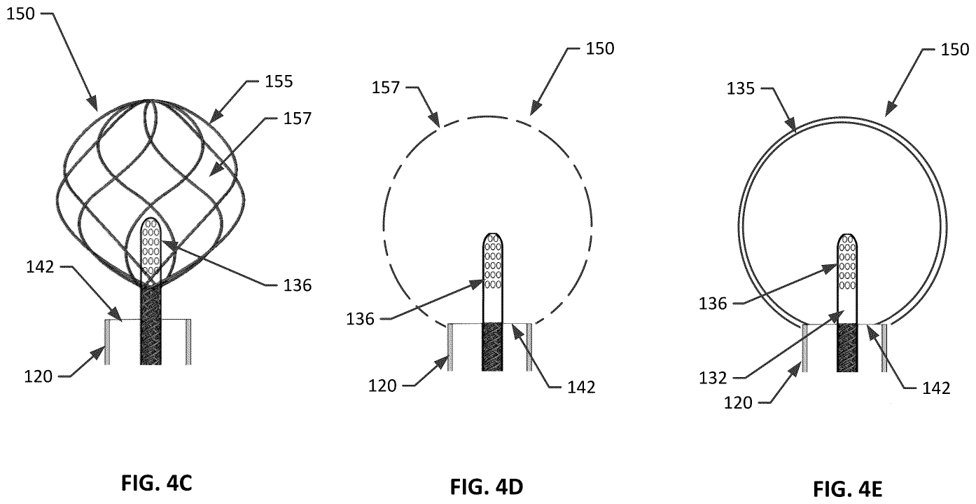

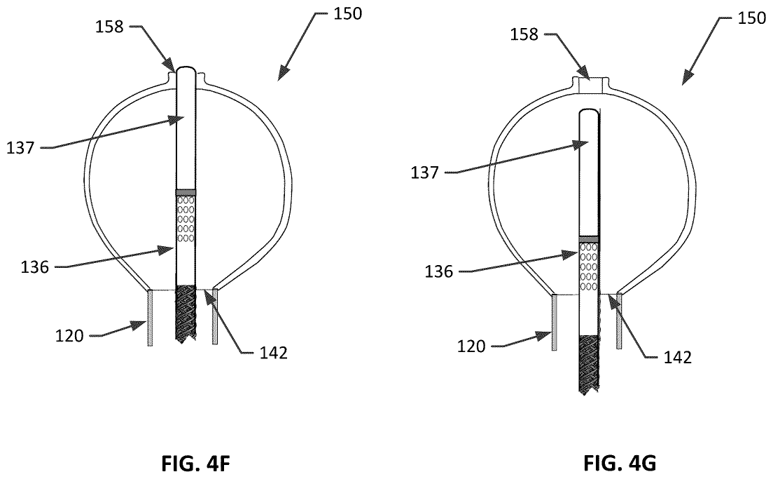

In other embodiments, described in more detail below with reference to FIG. 4E, the light scatterer 136 may be spaced from the light emitter 130, and instead be coupled to or form a portion of another structure, such as spacing member 150.

Fluid conduit 140 may be implemented with any known, suitable construction for conveying a fluid, such as saline, through catheter 110 to be discharged at the distal end of catheter body 120. The fluid may provide dilution, visualization, and/or cooling. For example, as shown schematically in FIGS. 3A and 3B, fluid conduit 140 may be an annular conduit defined between catheter body 120 and an inner body 148. Inner body 148 may provide a lumen or passage within which other structures, for example light conduit 132, may pass through catheter body 120. In the embodiment shown in FIGS. 3A and 3B, inner body 148 is a braided reinforcement or overwrap for optical fiber 132, to protect the delicate optical fiber and to provide a stiffer combined structure so that optical fiber, and attached scattering element 136, can be delivered distally through catheter body 120. Inner body 148 also supports light scatterer 136 on its distal end. In this embodiment, fluid conduit 140 is essentially the annular space left within the working channel 124 of catheter body 120 around inner body 148. In other embodiments, fluid conduit 140 may be arranged side-by-side with, rather than concentrically around, other structures such as light conduit 132. As shown schematically in FIG. 3A, outlet 142 may be configured simply as an opening at the annular distal end of the fluid conduit 140. In other embodiments, other geometries or structures may be employed to, for example, direct the flow of the dilution fluid laterally to the axis of catheter body 120, constrict the flow to reduce flow rate, accelerate the flow velocity, etc.

The inner body 148 of FIGS. 3A and 3B is shown in more detail in FIG. 3C. Inner body 148 has a distal portion 148A and a proximal portion 148B of different constructions. Proximal portion 148B may be formed as a solid tubular structure, e.g., a hypotube, that accounts for most of the length of inner body 148, e.g., 1 m, whereas the distal portion 148A may be formed as a braid or coil that accounts for a small portion of the length of inner body 148, e.g., about 25 cm. As described above, inner body 148 includes a central lumen that can receive the light conduit, such as optical fiber 132, and a distal tip on which the light scatterer 136 may be mounted. This construction provides a relatively stiff structure that enables the inner body 148 to be pushed distally through working channel 124 of catheter body 120, while the flexible distal portion 148A can be readily navigated into and through a tortuous body lumen BL. This embodiment is suitable for use with a catheter in which the spacing member 150 is coupled to the catheter body 110, as shown in FIGS. 4D-4G, described below. In an alternative embodiment, shown in FIG. 3D, spacing member 150 may be integrally formed with, or otherwise attached to the distal end 148A of inner body 148, as described in more detail below with reference to FIG. 4C.

Spacing member 150 may be implemented with a variety of structures and materials to provide the desired functions. A primary function of spacing member 150 is to maintain a minimum spacing between light emitter 130 and the treatment region TR, i.e., to prevent light emitter 130 from being disposed too close to treatment region TR, such that the light energy density of light emitter 130 is not above an acceptable upper limit. It may be further desirable for spacing member 150 to maintain a relatively uniform spacing between light emitter 130 and treatment region TR, for example keeping light emitter 130 relatively centered within body lumen BL. These related functions can be achieved in different ways. For example, in some embodiments, the light emitter 130 and spacing member 150 can be fixed relative to each other, and both can be moved through working channel 124 of catheter body 120 to the desired working position, as shown in FIG. 4C. In other embodiments, such as shown in FIGS. 4D and 4E, spacing member 150 may be fixed relative to catheter body 120, and light emitter 130 is movable relative to both.

As shown schematically in FIG. 4A, with spacing member 150 having a shape or geometry that is approximately symmetrical about the longitudinal axis LA of catheter body 120, and with light emitter 130 disposed approximately on longitudinal axis LA, light emitter 130 is approximately centered within spacing member 150. When the catheter 110 is disposed within a body lumen BL, and spacing member 150 is in an expanded configuration in which its diameter is close to the diameter of body lumen BL, light emitter 130 is approximately centered in body lumen BL. If the light emitted by light emitter 130 is then relatively uniformly distributed angularly around longitudinal axis LA, the light energy density on treatment region TR will be relatively uniform circumferentially; said another way, the light energy density on treatment region TR will be between desired upper and lower energy density values. Although schematically illustrated in FIG. 4A as being ellipsoidal in shape, spacing member 150 may be configured to be of any other shape desired, depending for example on the shape of the body lumen and/or treatment region. As shown schematically in FIG. 4B, if the body lumen is a sac-like shape, for example, an aneurysm, rather than tube-like, for example, a blood vessel, an ellipsoidal or spherical shape may be more appropriate to best center light emitter 130 within body lumen BL.

Another function of spacing member 150 may be to permit dilution fluid DF to pass therethrough and into body lumen BL. For example, as described in more detail below, it may be desirable for spacing member 150 to be filled with the dilution fluid DF, e.g., to displace blood or other body fluid BF, and/or to cause or aid in reconfiguration of spacing member 150 from a collapsed to an expanded configuration. It may also be desirable for the dilution fluid to dilute and/or displace blood or other body fluid between spacing member 150 and the treatment region TR and/or body lumen BL. It may also be desirable to use the fluid to distend or otherwise change the geometry or shape of the treatment region TR and/or the body lumen BL. Thus, the side wall of spacing member 150 may be porous or otherwise permeable to the dilution fluid. In some embodiments, the proximal end of spacing member 150 may surround all or part of the outlet 142 of fluid conduit 140, as shown schematically in FIG. 4B, in which case the dilution fluid will enter the interior of spacing member 150 and the fluid can be passed out of spacing member 150 (e.g., through porous or permeable walls of spacing member 150). In other embodiments, outlet 142 of fluid conduit 140 may be disposed outside of spacing member 150, and it may be desirable to permit the fluid to enter spacing member 150.

Another function of the spacing member 150 may be to distend or otherwise change the geometry or shape of the treatment region TR and/or the body lumen BL mechanically, i.e., by engaging the spacing member 150 with the surface of treatment region TR and/or body lumen BL.

Several possible constructions of spacing member 150 are shown schematically in FIGS. 4C to 4E. In the embodiment shown in FIG. 4C, spacing member 150 can be in the form of a wire cage that is formed of multiple wires or struts 155, defining numerous apertures 157 therebetween, through which dilution fluid discharged by fluid outlet 142 may pass. Spacing member 150 may be formed of a braided wire, or a laser cut tube, or of other known constructions. Spacing member 150 may be self-expanding, for example may be formed of a shape memory material such as Nitinol, set or biased to the expanded configuration but retained in the collapsed configuration, for example by being disposed within the fluid conduit 140 (or working channel 124) of catheter body 120. Alternatively, spacing member 150 may be biased to the collapsed configuration and require application of a force to urge it towards the expanded configuration.

In the embodiment shown in FIG. 4D, spacing member 150 is formed of an elastomeric material, and thus is essentially a balloon. However, rather than being a sealed balloon it includes perforations 157 to permit passage of dilution fluid, and thus is referred to as a “leaky” balloon. The proximal end of spacing member 150 is disposed around fluid outlet 142, and thus dilution fluid discharged from fluid outlet 142 enters the interior of spacing member 150 and may exit via perforations 157. The dilution fluid may be used to flush the interior of the balloon, and expel any air via a bleed channel or tube (not shown) before the light emitter is 136 is inserted through working channel 124 of catheter body 120 (not shown) distally into position within spacing member 150. Dilution fluid may also be used to inflate the balloon, i.e. to urge spacing member 150 from a collapsed configuration (not shown) to the expanded configuration shown in FIG. 4D. As the balloon expands, the perforations 157 expand, and thus provide a larger flow area for the dilution fluid to exit the balloon. Thus, the size of the balloon may be controlled by controlling the flow rate and pressure of the dilution fluid exiting fluid outlet 142. After the treatment procedure, dilution fluid may be withdrawn from the elastic balloon, allowing the spacing member 150 to move to a collapsed configuration in which it can be withdrawn from the patient's body.

In the embodiment shown in FIG. 4E, spacing member 150 is also formed of an elastomeric material, and thus is also essentially a balloon. However, in this embodiment, the balloon is sealed, i.e., it does not include perforations as with the previous embodiment. Fluid may be introduced into body lumen BL via a port through catheter body 120 (not shown). As with the previous embodiment, the interior of the balloon may be flushed, purged of air, and then may be expanded, i.e., the spacing member 150 may be urged from a collapsed configuration (not shown) to the expanded configuration shown in FIG. 4E, by introduction of dilution fluid from fluid outlet 142. Thus, the size of the balloon may be controlled by controlling the amount of dilution fluid that exits fluid outlet 142. In this embodiment, spacing member 150 also provides some or all of the function of light scattering, in that it includes on an inner surface or an outer surface of the spacing member 150, a layer 135 (shown in the inner surface in FIG. 4E) of light scattering material. Although FIG. 4E schematically also illustrates a light scatterer 136 on the tip of light conduit 132, in some embodiments, the layer of material 135 on spacing member 150 may be the only light scatterer. In some embodiments, light scattering can be provided by light scattering particles mixed or suspended in the dilution fluid. In some embodiments, the spacing member 150 can be at least partially transmissive and/or transflective of the light emitted from the light emitter 130.

The embodiment shown in FIGS. 4F and 4G is similar to that shown in FIG. 4E, except that spacing member 150 includes a fluid port 158 in the distal end thereof, and light scatterer 136 includes a valve extension 137 on its distal tip. Valve extension 137 can cooperate with fluid port 158 to selectively establish and prevent fluid communication between body lumen BL and the interior of spacing member 150. For example, the valve extension functions as a closure element and the fluid port 158 of spacing member 150 provides a distal closure surface such that when the valve extension 137 is moved distally, a tight seal is formed between the surface of port 158 and the surface of valve extension 137. Thus, as shown in FIG. 4F, with valve extension 137 disposed in fluid port 158 to fluidically isolate the interior of elastic spacing member 150, fluid discharged from fluid outlet 142 can expand spacing member 150 to the desired configuration. Valve extension 137 can then be withdrawn proximally, and thus disengaged from fluid port 158, as shown in FIG. 4G, to establish fluidic communication between the interior of spacing member 150 and body lumen BL. Fluid discharged from fluid outlet 142 can then be discharged via fluid port 158 into body lumen BL.

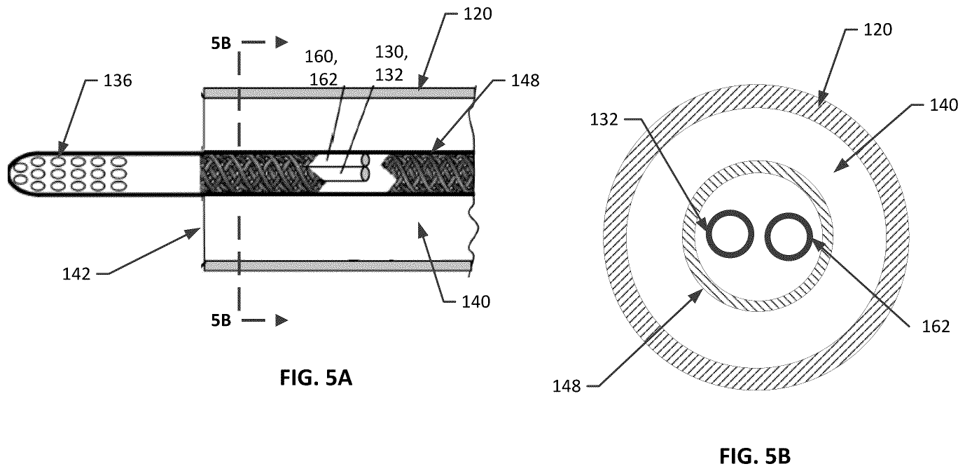

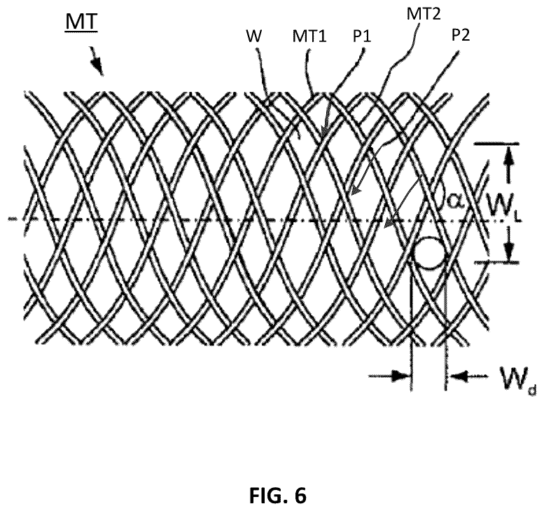

Imager 160 may be implemented with any known, suitable construction for collecting an image of treatment region TR or other portions of body lumen BL. Various imaging modalities may be employed, including optical (in wavelengths including visible, near infrared, and/or other portions of the spectrum), ultrasound, and optical coherence tomography (OCT). As shown schematically in FIGS. 5A and 5B, an imager 160 that is an optical imager in this example, may include an imaging conduit 162, for example an optical fiber. Imager 160 may provide a measurement of the light energy density applied to the treatment region TR by light emitter 130 over time, and thus the total energy applied. In other embodiments, imager 160 may enable acquisition of image information from treatment region TR, for example to aid in positioning of the distal end of the treatment system 100 relative to the treatment region TR, to evaluate the condition of the treatment region TR before, during, and after treatment, etc.

The light to be applied to treatment region TR, i.e., from light emitter 130, and optionally via light scatterer 136, may have wavelength(s) in the range of 400 nm to 1,100 nm. A convenient, and suitable, wavelength is 532 nm, which can be produced by readily available and inexpensive lasers and laser diodes. The power of the light applied to treatment region TR may be in the range from 1 mW to 500 mW, and preferably in a range of 100 mW to 200 mW. The power density of the light applied to treatment region TR may be in the range of 1 mW/cm² to 5 W/cm², preferably in a range of 50-500 mW/cm², and more preferably in a range of 175-200 mW/cm².

Although the mechanism of action is not well understood, it is believed that the application of light in the wavelengths and intensities described above may activate and/or accelerate hematopoiesis whereby stem cells differentiate to blood and blood vessel cells, which may then participate in rapid conversion of fresh thrombus to scar tissue and healing of the treatment region TR.

Mesh tube MT may be used in conjunction with catheter 110 in treatment of some indications and anatomical structures. Mesh tube MT may be of varied constructions, geometries, sizes, etc. suitable for the desired treatment. Examples of suitable mesh tubes are described in U.S. Pat. No. 7,942,925 to Yodfat et al., entitled “Implantable Intraluminal Device and Method of Using Same in Treating Aneurysms,” the entire disclosure of which is incorporated by reference herein. One suitable embodiment of mesh tube MT is shown schematically in FIG. 6, in an expanded configuration in which mesh tube MT would be disposed within a body lumen BL. Mesh tube MT includes a plurality of filaments of elastic or non-elastic bio-conpatible material, metal or plastic, extending helically in an interlaced manner to define a braided tube. Thus, a first group of filaments MT1 extend helically in one direction, and a second group of filaments MT2 extend helically in the opposite direction, with the two groups of filaments being interwoven such that a filament MT1 overlies a filament MT2 at some points as shown at P1, and underlies a filament MT2 at other points as shown at P2. Filaments MT1 and MT2 thus define a braided tube having a plurality of windows W. The inscribed diameter and the length of each window W are shown at W_{d} and W_{L}, respectively, in the implanted condition (e.g., expanded configuration) of the mesh tube MT. These characteristics depend on, among other factors: the number of filaments; the cross section of the filaments; and the implanted angle “a” at the cross-over points of the two groups of filaments MT1, MT2, Mesh tube MT may be disposed across the neck of a vascular aneurysm, along a straight section of a blood vessel or at or near a bifurcation of a blood vessel, and function to divert a portion of the blood flow through the vessel away from the aneurysm. The mesh tube MT can also be used to reduce blood flow to the selected part of a blood vessel in which blood coagulation is to be promoted. In some embodiments, the mesh tube MT is detached from the catheter 110 and deployed within the blood vessel. In some cases, the blood passing through the mesh tube MT into an aneurysm is subjected to a long residence time in the aneurysm, and therefore, platelets that have been activated during the passage into the aneurysm can initiate a thrombus formation that is now “jailed” or trapped inside the aneurysm. The light activation of the stem cells significantly accelerates the thrombus conversion to scar tissue and healing of the pathology.

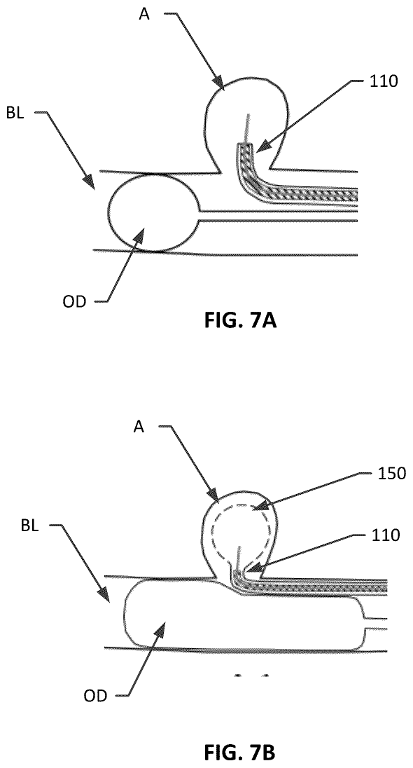

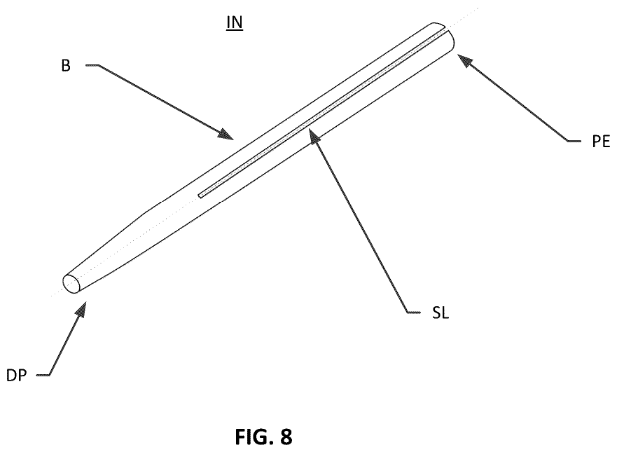

FIGS. 7A and 7B illustrate the use of an occlusion device OD that may be used in conjunction with catheter 110 in treatment of some indications and anatomical structures. As shown schematically in FIG. 7A, occlusion device OD may be placed in a body lumen BL, such as a blood vessel, and moved to an expanded configuration in which it engages the inner wall of body lumen BL and occludes it, i.e., reduces or prevents the flow of fluid, e.g. blood, through body lumen BL. In this embodiment, catheter 110 is shown with a distal end portion disposed in an aneurysm A upstream of occlusion device OD. As such, the catheter 110 can discharge fluid into aneurysm A and dilute blood therein to a desired dilution ratio without the fluid being carried away with blood through body lumen BL, since body lumen BL is occluded by occlusion device OD. In the embodiment in FIG. 7A, the distal tip of catheter 110 is steerable, and thus the light emitter may be disposed in a desired location within aneurysm A without the need for a spacing device. For delivery to the desired location in body lumen BL before treatment of the aneurysm A, and for withdrawal after conclusion of the treatment, occlusion device OD may be disposed in a collapsed configuration, and may be expanded to its expanded configuration to occlude body lumen BL by inflating the balloon with fluid, as is well known in the art. Suitable balloon occlusion devices include, for example, the HyperForm Occlusion Balloon distributed by Medtronic.

In another embodiment, shown in FIG. 7B, occlusion device OD may be an elongate balloon, such that it spans the neck of aneurysm A, and “jails” the distal tip of catheter 110. That is, the proximal portion of occlusion device OD can trap a portion of the distal end of catheter 110 against the wall of body lumen BL, thus immobilizing the catheter 110, in addition to occluding the lumen as with the previous embodiment. Suitable elongate balloon occlusion devices include, for example, the HyperGlide Occlusion Balloon distributed by Medtronic.

FIG. 8 illustrates an embodiment of an introducer IN that may be used in conjunction with catheter 110 in treatment of some indications and anatomical structures. Introducer IN has a generally cylindrical body B with a tapered distal portion DP, a proximal end PE, a central lumen extending through body B, and a slot SL in communication with the central lumen. The introducer IN can facilitate introduction of the inner body 148 into the catheter 110. For example, an incision or cutdown can be made in the patient's skin at the site where catheter 110 is to be introduced into the patient's body. Catheter 110 is then inserted into the patient's body through a standard introducer sheath. The proximal tip of catheter 110 is then fitted with a standard hemostatic valve. The tip of tapered distal portion DP can be inserted into the hemostatic valve, and introducer IN can be pushed into the hemostatic valve. When the distal end of introducer IN is in the desired position, the distal end of the inner body 148 can be inserted into the lumen of the introducer IN at proximal end PE and pushed through the lumen and out of the distal tip of introducer IN into the desired portion of the patient's anatomy, e.g., body lumen BL. Alternatively, the distal end of the inner body 148 can be preloaded into the lumen of the introducer IN

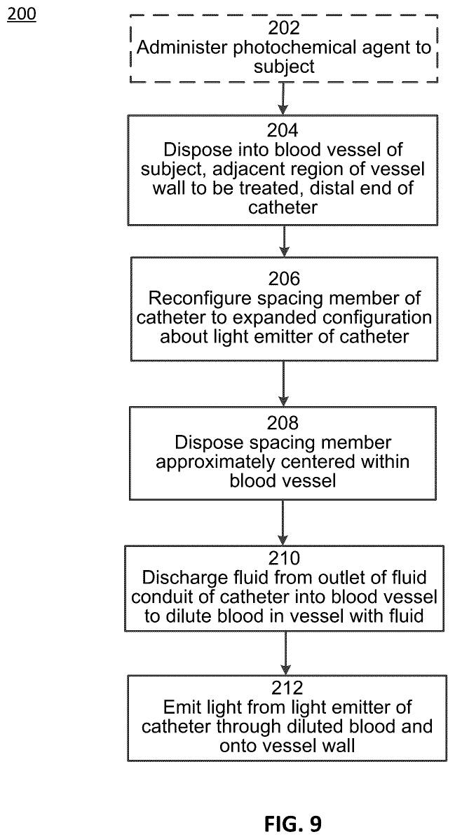

A method of treating a treatment region TR of a body lumen, in particular a portion of a lumen wall of a blood vessel, is illustrated schematically in FIG. 9. At 202, an optional photochemical agent can be administered to the subject (e.g., patient) to be treated. For example, a light-energy absorption agent, or a biochemical thrombosing agent, may also be applied to the interior of the selected part of the blood vessel to be treated, including the neck and all layers of the malformation. In some cases, a photochemical agent such as erythrosin B or rose bengal can be infused into the treatment region of the blood vessel before irradiation to enhance light absorption by the vascular wall and accelerate the photochemical reaction. The agent can be administered intravenously (IV) (i.e., systemically) or locally into blood vessel (or aneurysm or malformation) to be treated either through catheter 110 or via a separate microcatheter. Thereafter, an optical translucent or transparent field is established before the light energy is applied thereto.

At 204, a catheter (e.g., catheter 110) can be inserted into a blood vessel of the subject and a distal end of the catheter can be disposed adjacent or near a region of a vessel wall to be treated. In some embodiments, prior to inserting the catheter into the blood vessel, a guide wire is inserted into the blood vessel and positioned near the treatment region. The catheter can then be inserted over the guide wire (e.g., a lumen of the catheter can be received over the guidewire) and moved along the guidewire to the desired location at the treatment region.

At 206, a spacing member (e.g., spacing member 150) and a light emitter 130 (e.g., light emitter 130 with light scatterer 136) of the catheter 110 can be moved out a distal end of a lumen of the catheter 110, and the spacing member can be moved to an expanded configuration about the light emitter. The spacing member can prevent contact between the light emitter and the blood vessel wall and can ensure the centering of the light emitter within the blood vessel such that an even distribution of the photon energy flux to the surrounding vessel walls can be achieved. At 208, the spacing member can be positioned approximately centered within the blood vessel to be treated.

At 210, fluid can be discharged from an outlet of the fluid conduit of the catheter 110, and into the blood vessel to dilute blood within the blood vessel. For example, infusion of saline can be started to establish a clear or translucent optical field within the treatment region of the blood vessel. At 212, light energy can be emitted from the light emitter of catheter through the diluted blood and onto the wall of the blood vessel. The light energy can initiate and/or accelerate coagulation of blood therein within the treatment region, during which, the spacing member can prevent emboli, resulting from the coagulation, from moving through the blood vessel in the downstream direction. In some cases, the spacing member can be detached from the delivery system and remain permanently in the therapeutic site for protection. After treatment, the catheter can be removed from the blood vessel.

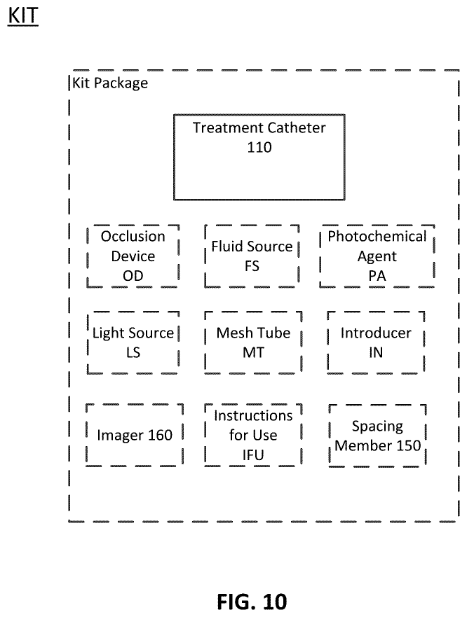

FIG. 10. is a schematic illustration of a kit, according to an embodiment. As described above, a treatment system 100 can be provided as a kit that includes one or more components to perform various functions to treat a treatment region TR. In some embodiments, a KIT can be a single use set of disposable components. In some embodiments, a KIT can include a catheter 110 disposed within a kit package, such as a sterile package used to protect the catheter 110 from contamination during transport and storage. In some embodiments, a kit package can include an outer package and one or more inner sterile packaging components to contain and protect one or more of the components of the KIT. The catheter 110 can include, for example, a catheter body 120 with a working channel 124, a light emitter 130, and a fluid conduit 140, as described above. A KIT can also optionally include one or more of each of the following components that can be used in conjunction with the catheter 110: an occlusion device OD, a light source LS, a fluid source FS, a photochemical agent PA, a mesh tube MT, an introducer IN, a spacing member 150, an imager 160, and/or instructions for use IFU. In some embodiments, a KIT can include, for example, multiple types of spacing member (e.g., 150) such that a user (e.g., physician) can select the appropriate spacing member 150 for a particular treatment. In some embodiments, a KIT can include, for example, multiple types of light emitter (e.g., 130) such that a user (e.g., physician) can select the appropriate light emitter 130 (e.g., with various types of a light scatterer 136, etc.), for a particular treatment. Each of the components of a KIT can be disposed within one or more sterile kit packages.

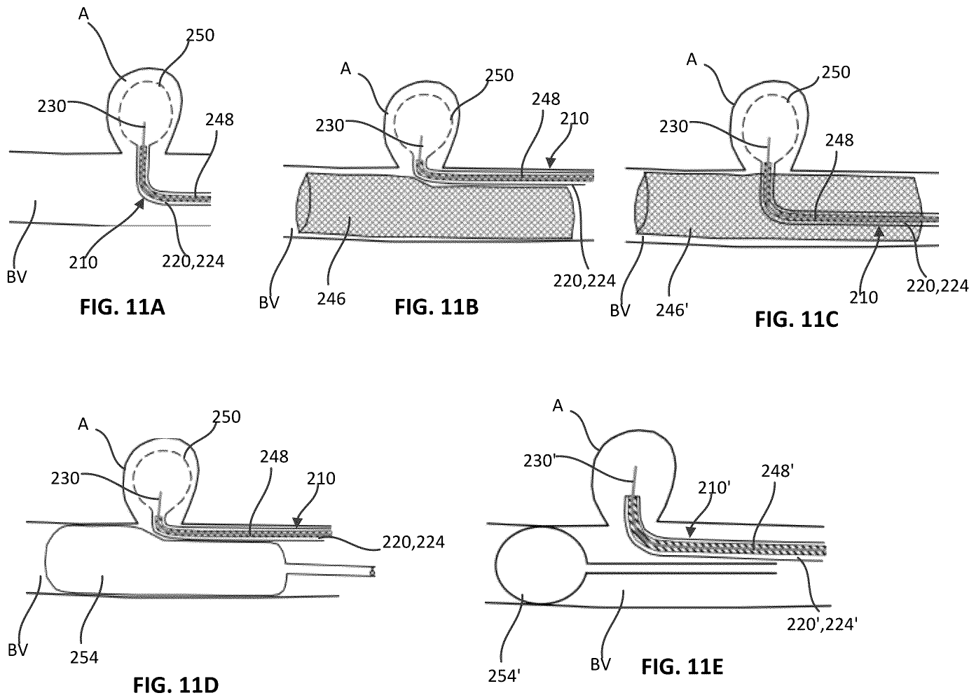

FIGS. 11A-11E illustrate various approaches to using treatment systems such as those described above to treat an aneurysm disposed laterally off a side wall of a blood vessel. The embodiments and particular components of the treatment systems shown and described with respect to FIGS. 11A-11E can be constructed the same as or similar to, and include the same or similar features as corresponding components of the system 100 described above. The system and components shown can be used to treat the aneurysm with, for example, light energy, as described above.

In the treatment approach shown in FIG. 11A, the treatment system includes a catheter 210 with a distal end portion of the catheter 210 disposed within an aneurysm A disposed laterally off a side wall of a blood vessel BV. The catheter 210 may include any of the features described above, the details of which are omitted from FIG. 11A for simplicity. For example, spacing member 250 disposed at the distal end of the catheter body 224 may be implemented with any of the options described above, include a porous balloon, a non-porous balloon, a wire cage, etc. The spacing member 250 can be moved between a collapsed configuration (not shown) during delivery to the treatment site and an expanded configuration (shown in FIG. 11A) during the treatment procedure using any of the techniques described above. The spacing member 250 can be used to maintain a minimum spacing between the light emitter 230 and the walls of the aneurysm A to be treated. With the light emitter 230 and spacing member 250 disposed within the aneurysm A, the light emitter 230 can be actuated to emit a desired light energy to treat the aneurysm A. Optionally, a fluid (e.g., saline) can be introduced into the treatment region as described above. Optionally, immediately following treatment (e.g., irradiation) of the aneurysm A with the light emitted from the light emitter 230, an intra-aneurysm implant, such as one or more coils, a Woven EndoBridge (WEB) device, or any other intra-aneurismal implant, can be deposited inside the aneurysm A, for example with the system in one of the configurations shown in FIGS. 11A, 11D and/or 11E. Alternatively, the spacing member 250 may be detached from the catheter body 224 and left inside the aneurysm A after the light treatment and withdrawal of the distal end of the catheter body 224 from the aneurysm A.

In the approach illustrated in FIG. 11B, the catheter 210 is being used in conjunction with a mesh tube 246. The mesh tube 246 can be formed and configured the same as or similar to the mesh tube MT described with respect to FIG. 6. The mesh tube 246 can be disposed within the blood vessel BV outside the aneurysm A such that it extends across (i.e., straddles) the opening of the aneurysm A. The mesh tube 246 can be deployed in a contracted or collapsed configuration and moved to an expanded configuration within the blood vessel BV. In this embodiment, the catheter 210 is inserted between the wall of the blood vessel BV and the mesh tube 246 prior to the mesh tube 246 being expanded. The mesh tube 246 is then expanded such that the mesh tube 246 holds or traps the catheter 210 against the wall of the blood vessel BV during treatment with the light emitter 230. A fluid (e.g., saline) can be introduced into the treatment region to establish a clear or translucent optical field and light energy is then applied via the light emitter 230 to irradiate the therapeutic target vessel wall to initiate or accelerate coagulation of the blood within the aneurysm. The mesh tube 246 can prevent emboli resulting from the coagulation from moving into the blood vessel. The mesh tube 246 can also function to divert a portion of the blood flow through the blood vessel BV away from the aneurysm A.

The approach illustrated in FIG. 11C is similar to that illustrated in FIG. 11B except that the catheter 210 is being used in conjunction with a mesh tube 246′, which can be formed and configured the same as or similar to the mesh tube MT described with respect to FIG. 6. The mesh tube 246′ can be disposed within the blood vessel BV outside the aneurysm A such that it extends across (i.e., straddles) the opening of the aneurysm A. In this approach, the mesh tube 246′ is expanded at the treatment location before the catheter 210 is inserted through a lumen defined by the mesh tube 246′ and inserted out a side wall of the mesh tube 246′ and into the aneurysm. The mesh tube 246′ can function to divert a portion of the blood flow through the blood vessel BV away from the aneurysm A. The mesh tube 246′ can also help maintain the position of the catheter 210 in relation to the aneurysm A during treatment with the light emitter 230. Fluid can be injected into the treatment region and light energy applied via the light emitter 230 as described above.

In the approach illustrated in FIG. 11D, catheter 210 is being used in conjunction with an occlusion device 254. The occlusion device 254 can be formed and configured the same as or similar to the occlusion device OD described above with respect to FIGS. 7A and 7B. For example, the occlusion device 254 can be an expandable balloon that can be moved between a collapsed configuration for delivery to the treatment location within the blood vessel BV, and an expanded configuration as shown in FIG. 11D. The occlusion device 254 can be disposed within the blood vessel BV in the collapsed configuration and deployed into the blood vessel outside the aneurysm A such that it extends across (i.e., straddles) the opening of the aneurysm A. The catheter 210 is inserted between the wall of the blood vessel BV and the occlusion device 254, and then the occlusion device 254 is moved to its expanded configuration such that the occlusion device 254 holds or traps the catheter 210 against the wall of the blood vessel BV. Fluid (e.g., saline) can be introduced into the treatment region to establish a clear or translucent optical field, and light energy is then applied via the light emitter 230 to irradiate the therapeutic target vessel wall to initiate or accelerate coagulation of the blood within the aneurysm A. During the light treatment, the occlusion device 254 prevents emboli resulting from the coagulation from moving into the blood vessel BV. The occlusion device 254 can also obstruct the flow of blood within the blood vessel during treatment of the aneurysm A.

In the approach illustrated in FIG. 11E, a catheter 210′ has a steerable distal end portion, and thus can be used without the spacing member 250. A distal end portion of the catheter 210′ is disposed within an aneurysm A as described for FIG. 11A with the light emitter 230 approximately centered within the aneurysm A by the steering control for the distal end portion. In this illustration, the catheter 210′ is being used in conjunction with an occlusion device 254′. The occlusion device 254′ can be formed and configured the same as or similar to the occlusion device OD described above with respect to FIGS. 7A and 7B. For example, the occlusion device 254′ can be an expandable balloon that can be moved between a collapsed configuration for delivery to the treatment location within the blood vessel BV to an expanded configuration as shown in FIG. 11E.

In this approach, the occlusion device 254′ is deployed into the blood vessel BV in a collapsed configuration to a position downstream of the aneurysm A to block the flow of blood through the blood vessel BV, and then expanded to fix the occlusion device 254 within the blood vessel distal of the aneurysm A. The catheter 210′ is inserted between an inflation conduit of the occlusion device 254′ and the wall of the blood vessel BV, and the distal end portion steered so that light emitter 230 is approximately centered in the aneurysm A. Fluid (e.g., saline) can be introduced into the treatment region to establish a clear or translucent optical field, and light energy is then applied via the light emitter 230 to irradiate the therapeutic target vessel wall to initiate or accelerate coagulation of the blood within the aneurysm A. During the light treatment, the occlusion device 254 also prevents emboli resulting from the coagulation from moving downstream into the blood vessel BV.

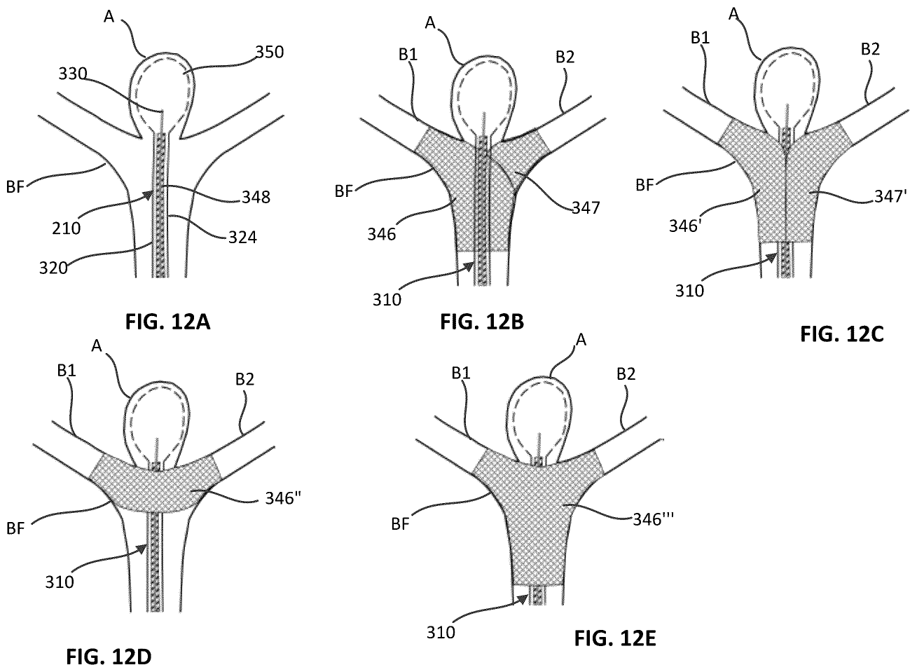

FIGS. 12A-12E illustrate various approaches to using treatment systems such as those disclosed above to treat an aneurysm disposed at a bifurcation in a blood vessel. The embodiments and particular components of the treatment systems shown and described with respect to FIGS. 12A-12E can be constructed the same as or similar to and include the same or similar features as corresponding components of the system 100 described above. The system and components shown can be used to treat the aneurysm with, for example, light energy, as described above.

In the treatment approach shown in FIG. 12A, the treatment system includes a catheter 310 with a distal end portion of the catheter 310 disposed within an aneurysm A disposed at a bifurcation BF of a blood vessel BV. The catheter 310 may include any of the features described above, the details of which are omitted from FIG. 12A for simplicity.

For example, spacing member 350 disposed at the distal end of the catheter body 324 may be implemented with any of the options described above, including a porous balloon, a non-porous balloon, a wire cage, etc. The spacing member 350 can be moved between a collapsed configuration (not shown) for delivery to the treatment location and an expanded configuration (shown in FIG. 12A) during treatment using any of the techniques described above. The spacing member 350 can be used to maintain a minimum spacing between the light emitter 330 and the walls of the aneurysm A to be treated.

The catheter 310 is inserted into the blood vessel BV and the distal end portion of the catheter 310, including the spacing member 350 and light emitter 330, are disposed within the aneurysm A. The spacing member 350 is moved to its expanded configuration and fluid is injected to establish a clear or translucent optical field. Light energy is then applied via the light emitter 330 to initiate and/or accelerate coagulation of blood at the treatment region. The spacing member 350 can help prevent emboli, resulting from the coagulation, from moving into the blood vessels in the downstream direction.

In the approach illustrated in FIG. 12B, the catheter 310 is being used in conjunction with a mesh tube 346 and a mesh tube 347 to further secure emboli from migrating into the blood vessels during treatment. The mesh tubes 346 and 347 can also serve as scaffold for vascular remodeling after photon therapy as well as serving as a filter for preventing emboli from migrating into the blood vessel. The mesh tubes 346 and 347 can be formed and configured the same as or similar to the mesh tube MT described with respect to FIG. 6. The mesh tube 346 includes a first portion that can be disposed within the blood vessel BV outside the aneurysm A and a second portion that extends into a branch B1 of the blood vessel BV. Similarly, mesh tube 347 includes a first portion that can be disposed within the blood vessel BV outside the aneurysm A engaging the mesh tube 346 and a second portion that extends into a branch B2 of the blood vessel BV. The mesh tubes 346 and 347 can be delivered in a collapsed configuration and moved to an expanded configuration at the treatment location. In this embodiment, the mesh tube 346 and the mesh tube 347 collectively define an elongate space between each other within the blood vessel BV and that terminates at the opening of the aneurysm. The distal end portion of the catheter 310 can be inserted between the vessel wall and the elongate space defined by the mesh tubes 346 and 347, effectively being “jailed” between the vessel wall and the mesh tube. Spacing member 350 and light emitter 330 are inserted into the aneurysm A, and the spacing member 350 can then be expanded within the aneurysm A and fluid injected to establish a clear or translucent optical field. Light energy is then applied via the light emitter 330 to initiate and/or accelerate coagulation of blood at the treatment region. The mesh tube as well as the spacing member 350 can also help prevent emboli, resulting from the coagulation, from moving into the blood vessels in the downstream direction.

The approach illustrated in FIG. 12C is similar to that illustrated in FIG. 12B, except in this illustration, the catheter 310 is being used in conjunction with a mesh tube 346′ and a mesh tube 347′, which can be formed and configured the same as or similar to the mesh tube MT described with respect to FIG. 6. The mesh tube 346′ includes a first portion that can be disposed within the blood vessel BV outside the aneurysm A and a second portion that can extend into a branch B1 of the blood vessel BV. Similarly, mesh tube 347′ includes a first portion that can be disposed within the blood vessel BV outside the aneurysm A and a second portion that can extend into a branch B2 of the blood vessel BV. In this embodiment, the mesh tube 346′ and the mesh tube 347′ abut each other within the blood vessel BV such that collectively the mesh tubes 346′ and 347′ are substantially Y-shaped (called the “double barrel technique”). In this embodiment, the catheter 310 is inserted and the distal end portion positioned within the aneurysm A, prior to the mesh tubes 346′ and 347′ being inserted into the blood vessel BV. After the catheter 310 is positioned, the mesh tubes 346′ and 347′ can be positioned within the blood vessel BV and branches B1 and B2 and expanded such that the mesh tubes 346′ and 347′ hold or trap the catheter 310 in the space formed between the mesh tubes 346′ and 347′ and the artery wall. The mesh tubes 346′ and 347′ can function to divert a portion of the blood flow through the blood vessel BV away from the aneurysm A. The mesh tubes 346′ and 347′ can also help maintain the position of the catheter 310 relative to the aneurysm during treatment with the light emitter 330.

In the approach of FIG. 12D, the catheter 310 is being used in conjunction with a single mesh tube 346″, which can be formed and configured the same as or similar to the mesh tube MT described with respect to FIG. 6. In this embodiment, the mesh tube 346″ includes a first portion that extends into the branch B1 and second portion that extends into the branch B2 of the blood vessel BV. The catheter 310 is inserted into the aneurysm and mesh tube 346″ is then opened to the expanded position essentially trapping the catheter 310 between the artery wall and the mesh tube 346″ with its distal tip inside aneurysm A as shown in FIG. 12D. The mesh tube 346″ can also help maintain the position of the catheter 310 relative to the aneurysm A during treatment with the light emitter 330.

In the approach illustrated in FIG. 12E, the catheter 310 is being used in conjunction with a single mesh tube 346′″, which can be formed and configured the same as or similar to the mesh tube MT described with respect to FIG. 6. The mesh tube 346′″ is substantially Y-shaped and includes a middle portion disposed within the blood vessel BV outside the opening of the aneurysm A, and two branch portions that extend into the branch B1 and branch B2 of the blood vessel BV. In this embodiment, the catheter 310 is inserted into the aneurysm A and then the mesh tube 346′″ is moved to the open position essentially “jailing” or trapping catheter 310 between the artery wall and the mesh tube 346′″ as shown in FIG. 12E. The mesh tube 346′″ can also help maintain the position of the catheter 310 relative to the aneurysm during treatment with the light emitter 330.

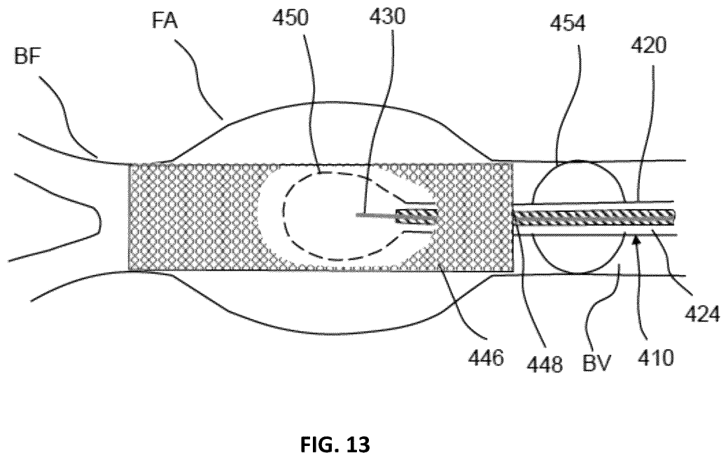

FIG. 13 illustrates the use of a treatment system, such as those described above, to treat a fusiform aneurysm FA disposed at a wall of a blood vessel BV near a bifurcation BF. The embodiments and particular components of the treatment system shown and described with respect to FIG. 13 can be constructed the same as or similar to and include the same or similar features as corresponding components of the system 100 described above. The system and components shown can be used to treat the fusiform aneurysm FA with, for example, light energy, as described above.

As shown in FIG. 13, the treatment system includes a catheter 410 shown with a distal end portion of the catheter 410 disposed within a fusiform aneurysm FA disposed at a side wall of a blood vessel BV. The catheter 410 may include any of the features described above, the details of which are omitted from FIG. 12A for simplicity.

The spacing member 450 may be implemented with any of the options described above, include a porous balloon, a non-porous balloon, a wire cage, etc. The spacing member 450 can be moved between a collapsed configuration (not shown) and an expanded configuration (shown in FIG. 13) using any of the techniques described above. The spacing member 450 can be used to maintain a minimum spacing between the light emitter 430 and the walls of the fusiform aneurysm FA to be treated. With the light emitter 430 and spacing member 450 disposed within the fusiform aneurysm FA, the light emitter 430 can be actuated to emit a desired light intensity to treat the aneurysm.

In this embodiment, the catheter 410 includes an occlusion device 454 coupled to the catheter body 420. The occlusion device 454 can be formed and configured the same as or similar to the occlusion device OD described above with respect to FIGS. 7A and 7B. For example, the occlusion device 454 can be an expandable balloon that can be moved between a collapsed configuration for delivery to the treatment location within the blood vessel BV and an expanded configuration as shown in FIG. 13. In this embodiment, the occlusion device 454 is disposed within the blood vessel BV ahead of the fusiform aneurysm FA to impede or to block the flow of blood through the blood vessel BV.

The catheter 410 can also be used in conjunction with a mesh tube 446, as shown for example in FIG. 13, which can be formed and configured the same as or similar to the mesh tube MT described with respect to FIG. 6. In this embodiment, the mesh tube 446 is elongate and is shown disposed inside the blood vessel BV traversing the fusiform aneurysm FA with “landing zones” in the artery on both ends of the aneurysm FA. The catheter 410 is inserted through a lumen defined by the mesh tube 446 and remains disposed within the lumen of the mesh tube 446 during treatment of the fusiform aneurysm FA with the light emitter 430.

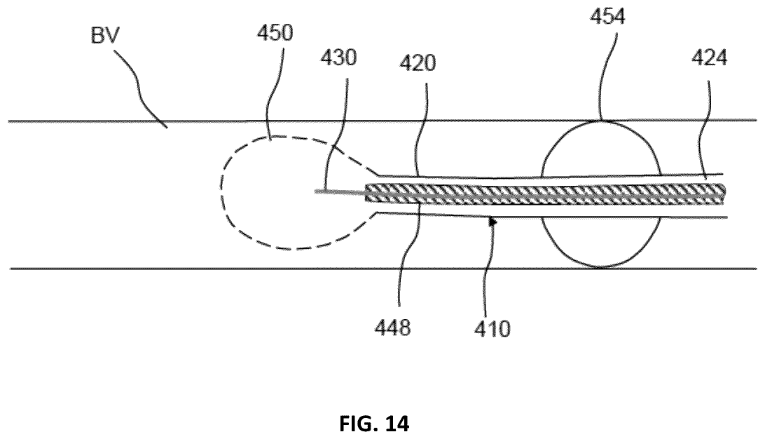

FIG. 14 illustrates the catheter 410 disposed within a blood vessel BV illustrating use of the catheter 410 for treatment of a wall of the blood vessel BV (for example, to treat a varicose vein) and using the occlusion device 454 to occlude blood flow within the blood vessel BV during treatment, for example, with the light emitter 430. Following the light treatment with the light emitter 430, devices suitable to occlude blood vessels, such as, for example, one or more thrombogenic coils or other suitable devices described herein, can be deposited inside the blood vessel BV to affect healing.

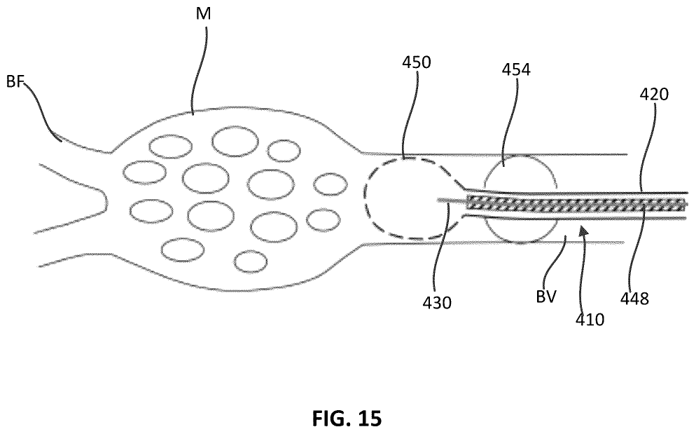

FIG. 15 illustrates the catheter 410 disposed within a blood vessel BV to treat a malformation M within the blood vessel BV. In FIG. 15, the bifurcation BF at the exit of the malformation M is illustrative of potentially multiple exits from the malformation M. In this example, the catheter 410 that includes an occlusion device 454 coupled to the catheter body 420 is positioned within the blood vessel BV leading directly into the malformation. Spacing member 450 is moved to the open position and fluid flow is initiated through flow channel 440. Irradiation of the malformation M is started by light emitted from the light emitter 430 to embolize the malformation within the blood vessel BV. Light penetration into the small frail arteries of the malformation M can initiate thrombus formation, followed by its conversion to scar tissue and can result in excluding the malformation M from the circulation. Following the light treatment with the light emitter 430, devices suitable to occlude blood vessels, such as, for example, one or more thrombogenic coils or other suitable devices described herein, can be deposited inside the blood vessel BV to affect healing.

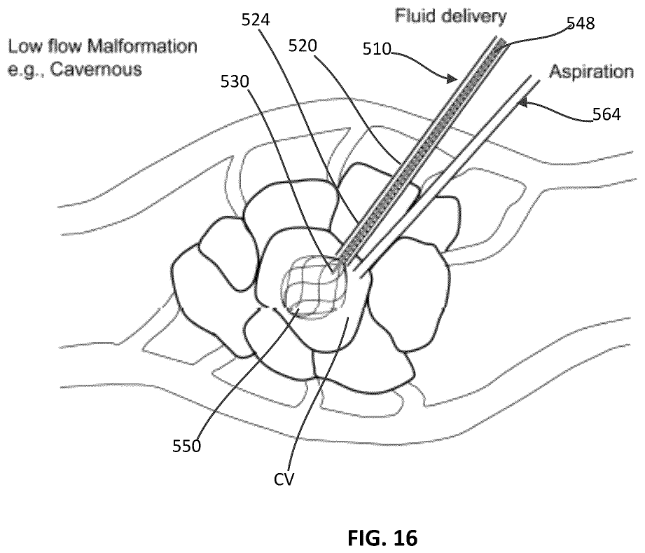

FIG. 16 illustrates components of a treatment system, such as those described above, being used to treat a low flow malformation, for example, a cavernous malformation within a subject's body. The embodiments and particular components of the treatment system shown and described with respect to FIG. 16 can be constructed the same as or similar to and include the same or similar features as corresponding components of the system 100 described above. The system and components shown can be used to embolize a malformation with, for example, light energy, as described above.

As shown in FIG. 16, the treatment system includes a catheter 510 shown with a distal end portion of the catheter 510 disposed within a cavernous region CV. The catheter 510 may include any of the features described above, the details of which are omitted from FIG. 16 for simplicity. In some embodiments, catheter 510 can be a blunt needle inserted over a trocar into the lesion.

For example, spacing member 550 is disposed at the distal end of the catheter body 520 through working channel 524. The spacing member 550 shown in FIG. 16 is constructed the same or similar to the spacing member 150 shown and described with respect to FIG. 4C. More specifically, the spacing member 550 includes multiple wires or struts that define numerous apertures therebetween through which dilution fluid discharged by the catheter 410 may pass. The spacing member 550 can be used to maintain a minimum spacing between the light emitter 530 and the walls of the treatment region. Any of the other spacing member designs described above can be used.

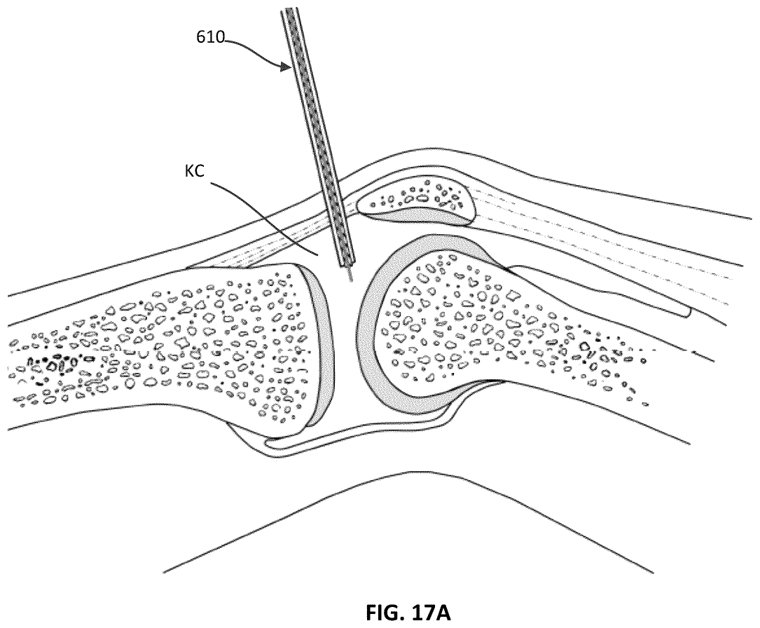

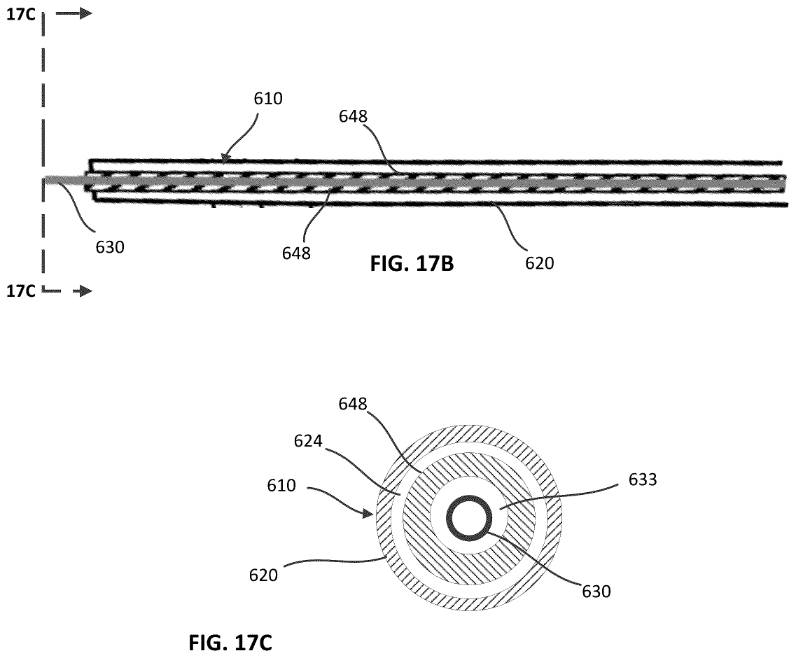

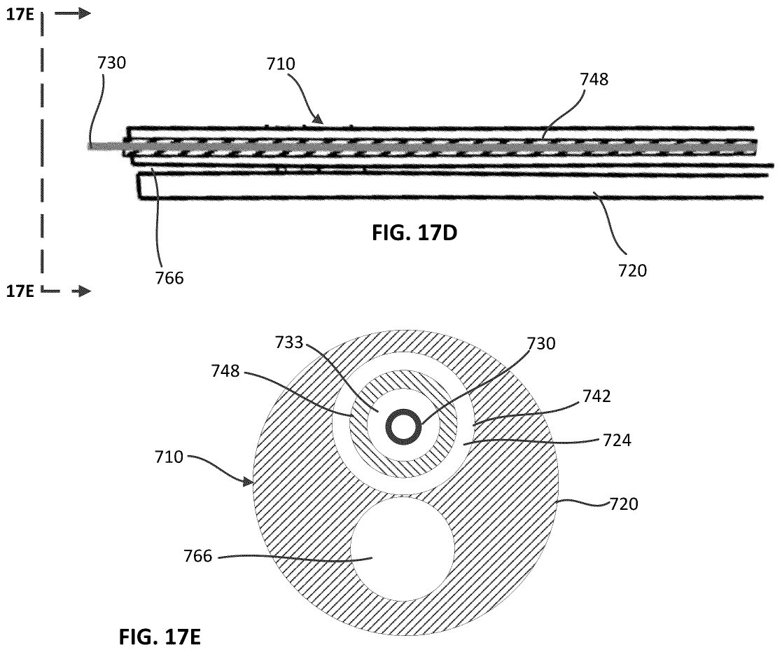

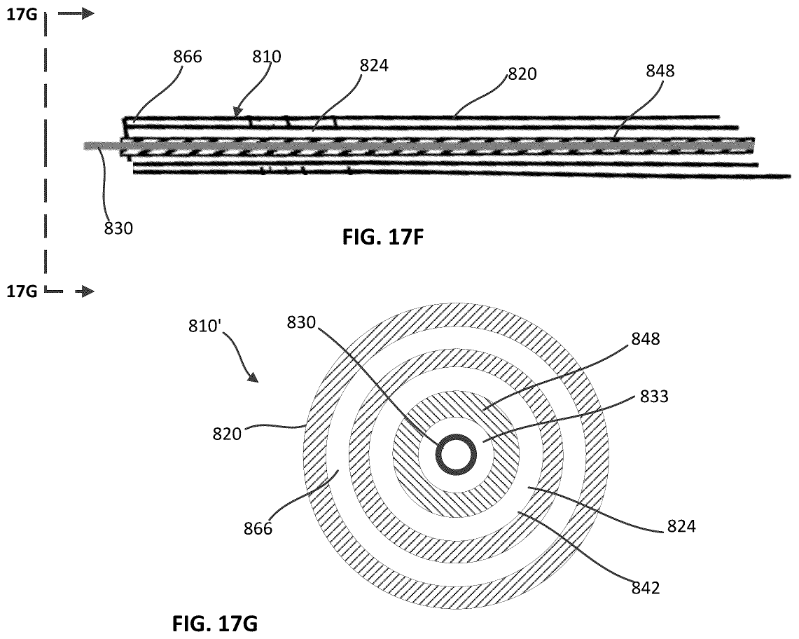

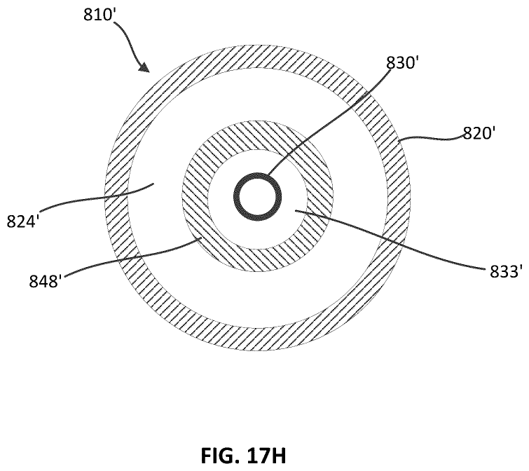

In this treatment approach, the catheter 510 is inserted into the cavernous malformation directly through the skin and intervening tissue of the patient (e.g., percutaneously), rather than intra arterially or intravenously. For example, the catheter 510 can be introduced into the patient's body via a delivery sheath inserted into the body through an opening in the skin and/or bony structures such as the skull of the patient's body. In this embodiment, the working channel 524 of the catheter body 520 can be used to introduce or inject fluid from a fluid source (not shown) into the treatment region, i.e., the cavernous region CV. A separate aspiration device 564 can be used to aspirate (e.g., remove) excess fluid and/or other material from the treatment region. In some embodiments, the catheter 510 can include an aspiration channel incorporated with the catheter body 524. Examples of such an embodiment are described below with respect to FIGS. 17A-17H.

FIGS. 17A-17C illustrate components of a treatment system being used to treat, for example, a cavernous malformation or a joint, e.g., a knee capsule KC (FIG. 17A). The embodiments and particular components of the treatment system shown and described with respect to FIGS. 17A-17C can be constructed the same as or similar to and include the same or similar features as corresponding components of the system 100 described above. The system and components shown can be used to treat the treatment region within the knee capsule KC with, for example, light energy, as described above.

As with the previous embodiment of FIG. 16, the treatment system is introduced directly (e.g., percutaneously) into the joint (e.g., knee capsule) of the patient, as shown in FIGS. 17A-17C. The treatment system includes a catheter 610 (e.g., a blunt needle) shown with a distal end portion of the catheter 610 disposed within a knee capsule KC. The catheter 610 includes a catheter body 620 that defines a lumen 624 through which an inner body 648 is movably disposed. The lumen 624 can also be used to introduce fluid (e.g., saline) from a fluid source (not shown) into the treatment region. In this embodiment, the inner body 648 defines a lumen 633 (see FIG. 17C) that can receive therein an optical fiber (not shown) coupled to a light emitter 630. Light scattering elements can be incorporated into light emitter 630 and attached to inner body 648. The light emitter 630 (and optical fiber) can be coupled to a light source (not shown). Since fluid in body joints is transparent or translucent, no dilution of such fluid with a transparent fluid such as saline is necessary in order to have the light irradiation reach the soft tissue to be treated. Further, if light scattering material that is fully transmissive, e.g., diamond dust, is used no cooling is required.

FIGS. 17D and 17E illustrate another embodiment of a catheter that can provide fluid flush and aspiration functions, and can be used, for example, to treat a joint, such as a knee capsule KC, as shown in FIG. 17A. The catheter 710 includes a catheter body 720 that defines a lumen 724 through which an inner body 748 is movably disposed. The inner body 748 defines a lumen 733 (see FIG. 17E) that can receive therein an optical fiber (not shown) coupled to a light emitter 730. Although not shown, light scattering elements can also be attached to or incorporated with light emitter 730. The light emitter 730 (and optical fiber) can be coupled to a light source (not shown).

In this embodiment, the lumen 724 of the catheter body 720 can also be used to introduce or convey a fluid, such as a saline, from a fluid source (not shown) into the treatment region, e.g., a cavernous malformation or a knee capsule KC. The fluid can exit the lumen 724 through a distal opening or outlet 742. As described previously, the fluid may provide dilution, visualization, and/or cooling within the treatment region. The catheter body 720 also defines a separate aspiration lumen 766 disposed parallel to the lumen 724. The aspiration lumen can be used to aspirate (e.g., remove) excess fluid from the treatment region, for example, either continuously during treatment or when the light treatment is completed.

FIGS. 17F and 17G illustrate another alternative embodiment of a catheter that can provide fluid flush and aspiration functions, and can be used, for example, to treat a joint, such as a knee capsule KC or a cavernous malformation, as shown in FIG. 17A and FIG. 16, respectively. The catheter 810 includes a catheter body 820 that defines a lumen 824 through which an inner body 848 is movably disposed. The inner body 848 defines a lumen 833 (see FIG. 17G) that receives therein an optical fiber (not shown) coupled to a light emitter 830. Although not shown, a light scattering element can also be attached to or incorporated with light emitter 830. The light emitter 830 (and optical fiber) can be coupled to a light source (not shown).

In this embodiment, the lumen 824 of the catheter body 820 can also be used to introduce or convey a fluid from a fluid source (not shown) into the treatment region, e.g., a cavernous malformation or a knee capsule KC. The fluid can exit the lumen 824 through a distal opening or outlet 842. The catheter body 820 also defines an aspiration lumen 866 that can be used to aspirate the treatment region (e.g., remove fluid and/or other material from the treatment region). In this embodiment, the lumen 824 (used for introducing fluid) and the aspiration lumen 866 are coaxial. Alternatively, the lumen 824 could be used for aspiration and the lumen 866 could be used for conveying fluid into the treatment region.

In an alternative embodiment shown in FIG. 17H, a catheter 810′ can include a catheter body 820′ that defines a lumen 824′ that can be used to both convey a fluid into a treatment region and aspirate the treatment region. For example, the catheter body 824′ can be coupled to a fluid source to convey fluid into the treatment region. The flow of fluid to the lumen 824′ can then be terminated and the catheter body 820′ can be coupled to a device to provide for aspiration through the lumen 824′. Alternately, lumen 824′ can be coupled to a valve that switches between a fluid irrigation source and an aspiration source.

As described above, in some embodiments, a system, such as any of the systems described herein, can be used to treat a target region (e.g., tissue) accessible from body cavities. For example, systems and methods described herein can be used to access body cavities containing cerebrospinal fluid (CSF) to target brain tissue and/or spinal disks. Additionally, systems and methods described herein can be used to access synovial joint cavities filled with synovial fluid to treat target regions of synovial joints.

In some embodiments, systems and methods described herein can be used to treat brain tissue damage resulting from an ischemic stroke, chronic neuropathic pain treatment, and/or a neurogenerative disorder (e.g., using photobiomodulation (PBM)). Neurodegenerative disorders that can be addressed include but are not limited to: Alzheimer's disease (AD) and other dementias; Parkinson's disease (PD) and PD-related disorders; prion disease; motor neuron diseases (MNDs); Huntington's disease (HD); spinocerebellar ataxia (SCA); spinal muscular atrophy (SMA); traumatic and non-traumatic spinal cord injury; seizure disorders; and neuropsychiatric conditions (e.g., anxiety and depression). Additionally, the systems and methods described herein can be used to induce neuroprotection, to cause neuro-regenerative effects (e.g., for ischemic and hemorrhagic stroke, and/or for improvement of cognitive function). For example, application of PBM via a Burr Hole (BH) or via a CSF space to ischemic damaged brain tissue in an acute stage following the ischemic event or in an already stroked brain tissue can generate and differentiate new neurons, enhance dendritic spine densities, foster new neuronal connectivity, and drive neuroplasticity. As another example, to treat chronic intractable neuropathic pain, PBM can be applied to the thalamus via a CSF space of the third ventricle and to the spinal cord via the anterior and posterior CSF space. As another example, to treat movement disorders, PBM can be applied to the deep brain (e.g., the thalamus, the subthalamic nucleus, and/or the caudate nucleus) through the CSF space of the ventricular system (e.g., the third and lateral ventricles).

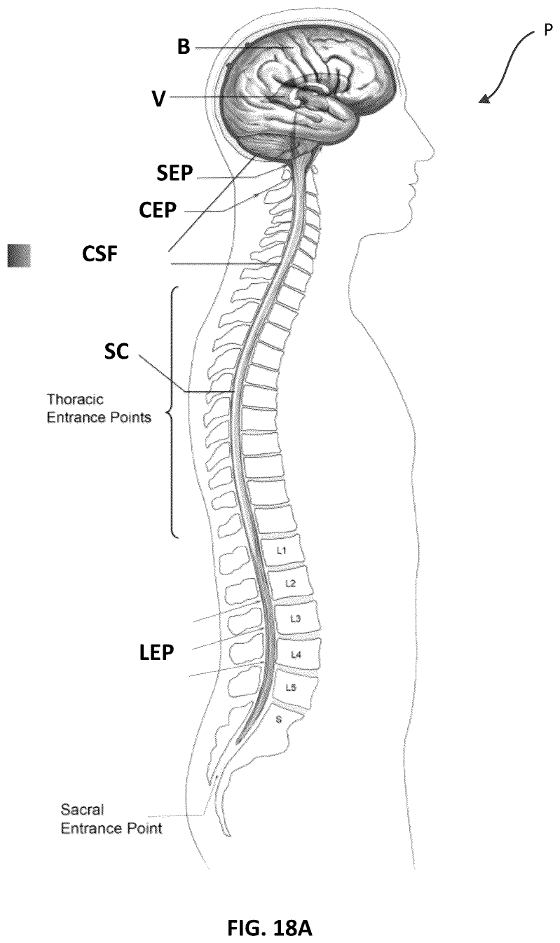

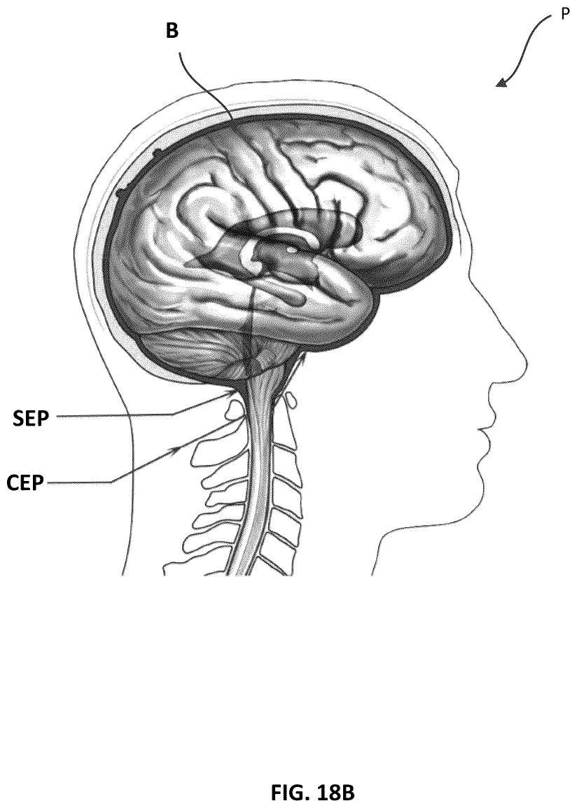

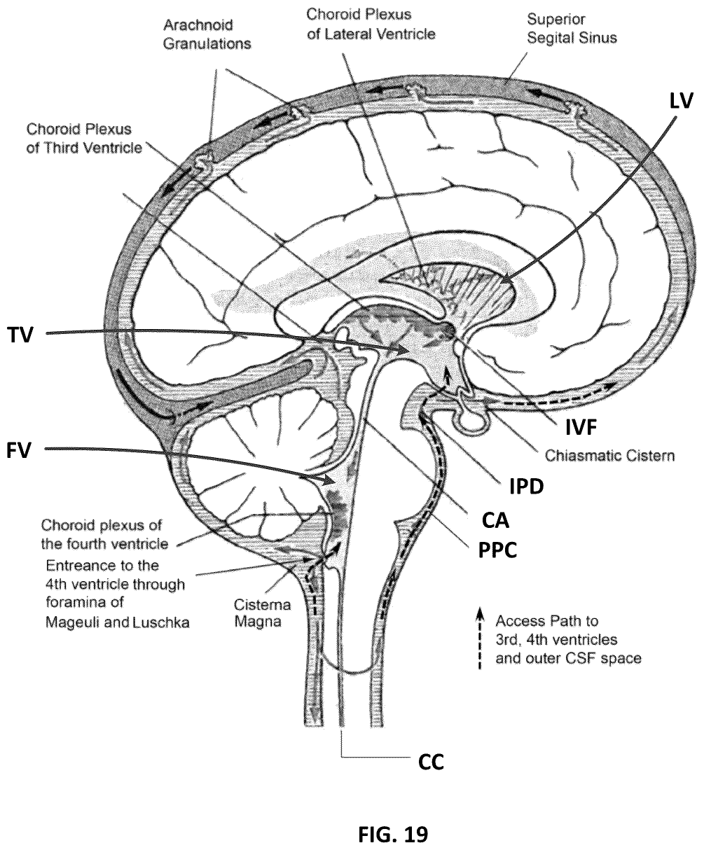

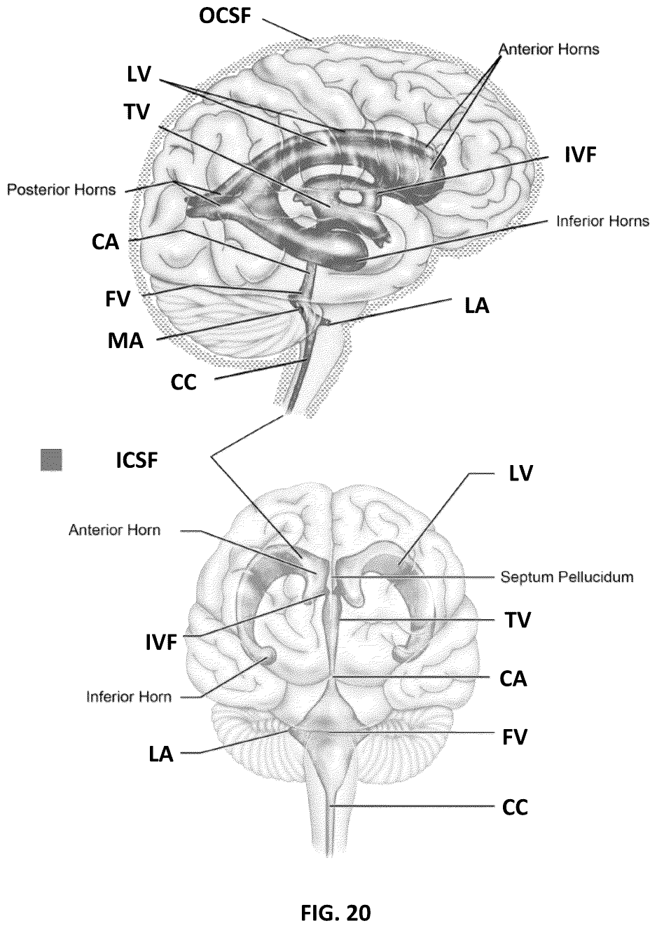

As described above, the CSF space is among the body cavities and spaces that can be accessed via the methods and systems described herein for treatment of tissue (e.g., brain tissue). FIGS. 18A-20 are various schematic illustrations of portions of a human brain, spinal cord, associated CSF spaces, and other related body portions. For example, FIG. 18A shows a patient P having a brain B and spinal cord SC. As shown in FIGS. 18A-20, CSF space surrounds the inner parts of the brain (e.g., filling the ventricles V and central canal CC), the outer parts of the brain B, the brain surface, and the spinal cord SC (e.g., by circulating within the spinal canal). Specifically, the CSF space includes an outer CSF space OCSF that surrounds the brain convexity, fills fissures (e.g., Sylvian fissure), and includes the spinal canal, as well as surrounds brain inner surfaces (e.g., filling the basal cistern, the interpeduncular cistern IPD, the pre pontine cistern PPC, etc.). The CSF space also includes inner CSF space(s) ICSF including a closed ventricular system which is a closed CSF space within the brain, as shown in FIGS. 18A, 18B, and 19. The ventricular system includes four ventricles (i.e., two lateral ventricles LV, a third ventricle TV, and a fourth ventricle FV). The third ventricle TV is connected to both lateral ventricles LV via an inter-ventricular foramen IVF (foramen of Monroe) and to the fourth ventricle FV via a cerebral aqueduct CA. The CSF space continues from the fourth ventricle FV into the central canal CC within the spinal cord SC. The fourth ventricle is coupled with the outer CSF space OCSF (including the spinal canal) and the exit site for CSF through lateral apertures LA (i.e., the foramina of Luschka, which are paired apertures located in the lateral recesses of the fourth ventricle) and a median aperture MA (i.e., the Foramen of Magendie) as shown in FIG. 20.

In some embodiments, the CSF space can be accessed surgically (e.g., via a burr hole or a craniectomy). In some embodiments, the CSF space can be accessed via a minimally invasive approach (e.g., using a micropuncture kit at cervical, thoracic, or lumbar spine level). In some embodiments, the CSF space can be accessed using a pre-existing access site. One or more catheter(s) can be navigated (e.g., steered over one or more wires) through the access site to a target location (e.g., within the CSF space and/or within brain tissue). In some embodiments, the target location can be accessed via the spinal canal. When in the target location, PBM therapy (e.g., light) can be emitted from the one or more catheter(s) to a target treatment area. For example, an electric and/or light carrying device (e.g., an optical fiber device) can be coupled to or inserted through the one or more catheter(s) to the target location (i.e., the treatment area), and PBM therapy can be delivered from the electric and/or light carrying device to a target treatment region (e.g., target tissue) associated with (e.g., aligned with or therapeutically accessible from) the target location. In some embodiments, the PBM therapy can be delivered from the distal end of an optical fiber and/or from miniature LEDs of the electric and/or light carrying device. The PBM therapy can be used to stimulate, regenerate, and/or heal injuries of the brain and/or spinal cord and/or to prevent further injury, e.g., in acute ischemic stroke of brain and spinal cord or traumatic brain injury (TBI). PBM therapy can also be used to reduce inflammation and relieve both acute and chronic neurogenic pain.

In some embodiments, for treatment via the CSF space using any of the systems and methods described herein, the CSF space of a patient can be accessed at any point of the spinal axis. For example, in some embodiments, the access location can be at the lumbar spine at the back and below the spinal cord SC. Various example lumbar CSF entrance points are represented as LEP in FIG. 18A. In some embodiments, the access location to the CSF space can be via a suboccipital approach (represented as SEP in FIGS. 18A and 18B) or at the cervical vertebral body at level C1/C2 (represented as CEP in FIGS. 18A and 18B) following a puncture with a fine needle (see, e.g., FIGS. 18A and 19). In some embodiments, the CSF space can be accessed directly after creating a burr hole in the head of the patient or via a craniectomy. Once access to the CSF space is created, in some embodiments, a guide sheath (e.g., a tube) can be placed either temporarily or permanently through the access and secured to the skin (e.g., in a sterile fashion). Steerable catheters can then be navigated through the guide sheath (e.g., with or without the aid of a guidewire) through the entrance point in the CSF space (e.g., anterior to the spinal cord) to the area of interest (i.e., target location) along the spinal cord SC or the brain surface defining the CSF space (e.g., brain convexity, fissures, and/or cisterns).

For example, in some embodiments, for treatment via the ventricular system (i.e., inner CSF spaces) using the systems and methods described herein, access to the ventricular system can be obtained either through use of a burr hole or via a craniectomy. A guide sheath (e.g., tube) can be placed through the brain tissue into a lateral ventricle LV. In some embodiments, CSF access can be gained via a path traveling through the CSF space along the spinal cord SC after placement of a sheath as described above. A steerable catheter can be navigated along or over a guidewire anterior of the spinal cord SC apically via the prepontine cistern PPC and interpeduncular cistern IPD to the floor of the third ventricle TV (shown in dashed lines in FIG. 19). The third ventricle TV can be accessed using a wire, a balloon, mechanical instruments, and/or laser light to create a fenestration (also referred to as a window) in the floor of the third ventricle TV (i.e., a ventriculostomy). A catheter, such as any of the catheters of the systems described herein, can then be advanced through the fenestration. In some embodiments, the catheter can be navigated to a target location (e.g., to and through the fenestration) using fluoroscopy and a road map generated after injecting dye into the CSF space. In some embodiments, the catheter can be navigated to a target location using a segmentation process based on MRI and/or CT images. For example, the catheter can be navigated to a target location (e.g., region or area) within the ventricular system and adjacent to various deep nuclei involved in neurodegenerative disorders, such as caudate nucleus, thalamus, subthalamic nucleus. In some embodiments, once the third ventricle TV is accessed, and the steerable catheter is placed at the target location within the CSF space or advanced into the brain tissue, light can be emitted from the catheter to therapeutically treat target tissue via any of the methods described herein. For example, fiber optic element(s) can be advanced relative to the catheter (e.g., through a lumen or channel of the catheter, such as co-axially) and/or activated to deliver low-level laser therapy (LLLT) to a target tissue.

In some embodiments, the ventricle system can be accessed through CSF space behind the spinal cord SC via the Foramen of Magendie MA or the foramina of Luschka LA into the fourth ventricle FV (see, e.g., dashed lines in FIG. 19). The third ventricle TV and the lateral ventricles LV can be accessed directly from the fourth ventricle FV via the cerebral aqueduct CA (i.e., the Aqueduct of Sylvius) and foramen of Monro IVF, respectively.

In some embodiments, systems and methods described herein can be used to treat patients having a chronic subdural hematoma (cSDH). As described above, the pathophysiology involved in cSDH appears to be a result of an initial injury to the dural border cell layer on the inner surface of the dura, which tears and leads to the extravasation of cerebrospinal fluid and blood in the space between the broken cell layer and the rest of the dura. The injured cells activate inflammatory mediators including interleukins and other cytokines that recruit inflammatory cells. This cascade induces the release of vascular growth factors including vascular endothelial growth factor, cyclooyxygenase-2, transforming growth factor-β1, and platelet-derived growth factor, which have been demonstrated to be heavily concentrated in this fluid. More inflammatory cells are recruited into the cavity, producing ongoing cell injury, and further stimulating inflammatory cells and angiogenetic factors. As described above, the initial injury to the dural border cell layer in some patients is unable to be repaired and thus stimulates a cycle of hyperfibrinolysis, inflammation, angiogenesis, and the resultant development of subdural neo-membranes.

In some embodiments, PBM can be applied to address this problem associated with cSDH using different biological pathways. For example, in some embodiments, systems and methods described herein can be used to dispose a light emitter between an inner and outer membrane of a brain, between a neo-membrane and dura mater of the brain, and/or between a dural border cell layer and a remainder of the dura (e.g., within the cSDH cavity) and used to emit light to the cSDH cavity and/or adjacent tissue regions to treat patients having cSDH. In some embodiments, low-level laser light therapy can be directly delivered into the cSDH cavity and the photons can dissociate inhibitory nitric oxide from the enzyme, leading to an increase in electron transport, mitochondrial membrane potential and ATP production. In some embodiments, light-sensitive ion channels can be activated allowing calcium to enter the cell. After the initial photon absorption events, numerous signaling pathways can be activated via reactive oxygen species, cyclic AMP, NO and Ca2+, leading to activation of transcription factors. These transcription factors can lead to increased expression of genes related to protein synthesis, cell migration and proliferation, anti-inflammatory signaling, anti-apoptotic proteins, and antioxidant enzymes. Stem cells and progenitor cells appear to be particularly susceptible to PBM. In some embodiments, these cellular pathways in the cavity wall and in the fluid within can be activated to initiate recruitment of remote cellular elements to repair (e.g., seal) the cavity. In some embodiments, an optical cable can be used to occlude the MMA through the interaction of the light energy with the cellular elements of the vascular wall (e.g., as shown in FIG. 4A or FIG. 14). In some embodiments, such as is shown in FIG. 30, electric conduits imbedded in the optical cable can be used to initiate a slight thermal injury to the vascular wall (e.g., without detaching a spacing member such as the spacing member 1150), and then low level light therapy can optionally be applied to stimulate reparative pathways of the patient to induce an injury repair response.

In some embodiments, a system, such as any of the systems described herein, can include a catheter device that can include a slender tubular body and/or an optical cable, with a distal end, a proximal end, and a light emitter disposed at the distal end (e.g., of the optical cable) and configured to emit light. The optical cable can include, for example, one or more optical fibers configured to deliver laser light and/or one or more electric wires coupled to light emitting diodes (LEDs) and/or light detectors. A spacing member can be disposed at or near the distal end of the optical cable and is reconfigurable from a collapsed configuration to an expanded configuration. In the expanded configuration, the spacing member can be disposed about the light emitter to maintain the light emitter approximately centered within the spacing member with respect to at least one axis of the spacing member. The spacing member can be at least partially transmissive and/or transflective of the light emitted from the light emitter. The catheter device can be configured such that the distal end of the catheter body can be inserted at least partially into a body cavity or lumen having an interior wall, the spacing member can be transitioned to the expanded configuration within the body cavity or lumen (e.g., into contact with the interior wall but without applying sufficient pressure to the interior wall to expand the body cavity or lumen), and light can be emitted from the light emitter to illuminate the interior wall of the body cavity or lumen. In some embodiments, the light emitter may be or have a distal end that is configured to deliver the light in a lateral or side trajectory, rather than axially down the cable, such that the light emitter can illuminate a region of the interior wall that is on only one side of the light emitter and/or catheter (e.g., a minor arc portion relative to the emitter), rather than illuminating a region around the entire circumference of the light emitter and/or catheter.

In some embodiments, a method includes disposing a distal end of a catheter into a body lumen (also called a passageway) or a body cavity (e.g., a blood vessel, an airway duct, urinary tract, CSF tract or other body cavity) of a subject adjacent to a region of a wall of the vessel or body cavity to be treated. For example, the distal end of the catheter can be disposed within any of the body lumens or cavities described herein using any of the access procedures described herein. The distal end of the catheter can be disposed at the center or proximal end of a target treatment area or region. The catheter can include (e.g., disposed at the distal end thereof): a light emitter configured to emit light; optionally, an outlet of a fluid conduit coupled to a source of fluid; optionally, an inlet of a fluid conduit coupled to a fluid sink; and, optionally, a spacing member reconfigurable from a collapsed configuration to an expanded configuration. The spacing member can be at least partially transmissive and/or transflective of the light emitted from the light emitter, and porous to fluid discharged from the fluid outlet. The spacing member can be transitioned to the expanded configuration when disposed at the treatment location. When in the expanded configuration, the spacing member can be disposed about the light emitter to maintain the light emitter approximately centered within the spacing member with respect to at least one axis of the spacing member. The method further includes disposing the spacing member approximately centered within the vessel lumen or body cavity and/or disposing the light emitter a predetermined distance from target tissue based on the diameter of the spacing member in the expanded configuration. In some embodiments, a fluid can be discharged from the outlet of the fluid conduit into the vessel or body cavity to cool the light emitter. In some embodiments, such as if blood is disposed in the target treatment area or region (e.g., a vessel or body cavity), fluid can be discharged from the outlet of the fluid conduit into the vessel of the body cavity to dilute the blood with the fluid to establish a translucent or transparent optical domain (also referred to as an optical field). In some embodiments, such as if the body fluid in the body cavity is clear (e.g., CSF fluid), fluid can be discharged from the body cavity via a fluid sink (e.g., proportionally to any fluid added via a fluid conduit to maintain a pressure of the body cavity within a threshold range). Light can be emitted from the light emitter through diluted blood (e.g., in a blood vessel lumen) or through optically clear fluid (e.g., in a body cavity) and onto the target tissue (e.g., the region of the wall to be treated).

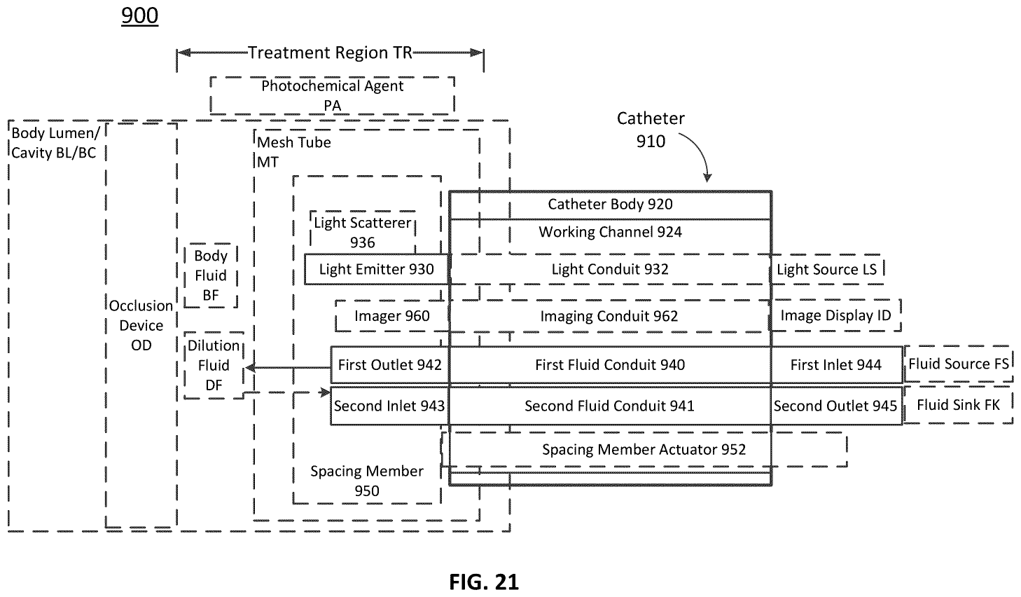

In some embodiments, as illustrated schematically in FIG. 21, a treatment system 900 can include a catheter 910, which may be operatively coupled to other devices or systems, including a light source LS, a fluid source FS, a fluid sink FK, and/or an image display ID, and may be used in conjunction with other devices, including a mesh tube MT, a helical coil, an occlusion device OD, and an introducer (not shown in FIG. 21), and with compositions such as a photochemical agent PA. The treatment system 900, and components thereof, can be the same or similar in structure and/or function to any of the treatment systems described herein, such as the treatment system 100. For example, the catheter 910 can be the same or similar in structure and/or function to any of the catheters described herein, such as the catheter 110.

Catheter 910 can have an elongate catheter body 920 with a proximal end and a distal end suitable for insertion into a body lumen BL or body cavity BC. The body lumen BL or body cavity BC can include, for example, a blood vessel, a synovial joint, or a CSF-filled space near (e.g., adjacent) to or including a treatment region TR of the body lumen BL or cavity BC. In some embodiments, as described above, the body lumen BL or body cavity BC and/or the treatment region TR can include a cSDH cavity. Catheter body 920 may define an internal working channel 924, in which other components of catheter 910 can be disposed, and may be moveable therethrough. Thus, in some embodiments, catheter body 920 may be inserted into the body of the patient until the distal end is disposed adjacent to the treatment region TR. In some embodiments, a distal end of the catheter body 920 can be translated to a body cavity BC of the patient (e.g., a cavity filled with CSF) via any of the access locations and access procedures described herein (e.g., described with respect to FIGS. 18A-20). For example, in some embodiments, the catheter body 920 can be delivered over a guidewire through the vasculature of the patient. The guidewire can be removed and one or more other components of catheter 910 can be delivered through the working channel 924 until the distal end(s) are disposed within the treatment region TR in appropriate working relation to the distal end of catheter body 920 and target tissue. In other embodiments, some or all of the other components of catheter 910 may be disposed in and/or coupled to catheter body 920 before catheter 910 is inserted into the body of the patient and the distal end delivered to the treatment region TR.

The catheter 910 includes a light emitter 930, which is disposed at the distal end of the catheter body 920 when the catheter 910 is configured for use. In some embodiments, the light emitter 930 can be translated to a location distal of the distal end of the catheter body 920 (e.g., through the working channel 924 prior to or after disposing of the distal end of the catheter body 920 in the target region TR). In some embodiments, the light emitter 930 can be coupled to the distal end of the catheter body 920 prior to disposing the distal end of the catheter body 920 in the target region TR. The light emitter 930 may be optically coupled to the light source LS by a light conduit 932, which may be disposed within catheter body 920 (e.g., in working channel 924) and may extend from the proximal to the distal end of the catheter body 920. The light conduit 932 can include, for example, one or more optical and/or electrical cables.

The catheter 910 optionally includes a first fluid conduit 940, which may be disposed within catheter body 920 and extend from a first inlet 944 at the proximal end of catheter body 920 to a first outlet 942 at the distal end of catheter body 920. The first fluid conduit 940 may be coupled to the fluid source FS via the first inlet 944. In some embodiments, the first fluid conduit 940 is defined by the catheter body 920. In some embodiments, the first fluid conduit 940 can be formed as a tube configured to be inserted through a lumen defined by the catheter body 920, such as the working channel 924. In addition to the first fluid conduit 940, catheter 910 can optionally include a second fluid conduit 941. The second fluid conduit may be disposed within catheter body 920 and can extend from a second inlet 943 at the distal end of catheter body 920 to a second outlet 945 at the proximal end of catheter body 920. The second fluid conduit 941 may be coupled to the fluid sink FK via the second outlet 945. Thus, fluid can be provided from a fluid source FS to a region distal of the catheter 910 via the first fluid conduit 940 and can be withdrawn from the region distal of the catheter 910 into the fluid sink FK via the second fluid conduit 941. Thus, optionally, a pressure can be maintained within the treatment region TR (e.g., within the body cavity BC) within a range (e.g., a safe range) via at least one of providing fluid to the treatment region TR or drawing fluid form the treatment region TR. The pressure can be maintained to maintain an equilibrium pressure at the treatment region TR (e.g., maintain a natural pressure of the treatment region TR) during the treatment process. For example, if fluid is introduced to the treatment region TR (e.g., infused) from the fluid source FS to cool the light emitter 930 and/or to dilute fluid in the treatment region TR (e.g., if the treatment region TR includes blood) to establish a translucent or transparent optical domain (also referred to as an optical field), fluid can be discharged from the treatment region TR via the fluid sink FK (e.g., proportionally to any fluid added via the first fluid conduit 940) to maintain a pressure of the treatment region TR at an equilibrium pressure or within a threshold range or the equilibrium pressure of the treatment region TR.

The catheter 910 may also include a spacing member 950 disposed at the distal end of the catheter body 920. The spacing member 950 can be the same or similar in structure and/or function to any of the spacing members described herein. The spacing member 950 can be actuated from an initial configuration to an expanded configuration in which the spacing member 950 has a larger diameter and/or lateral extent than in the initial configuration. The initial configuration can be, for example, a collapsed and/or uninflated configuration. When in the expanded configuration, the spacing member 950 can be configured to center the light emitter 930 relative to at least one axis of the spacing member 950 (e.g., relative to one, two, or three axes of the spacing member 950), to center the light emitter 930 relative to opposing walls defining the cavity or passageway of the treatment region TR, and/or to maintain the light emitter 930 at a predetermined distance from the target tissue of the treatment region TR (e.g., a predetermined distance from a target portion of the wall). In some embodiments, the spacing member 950 can be formed of a porous material, such as a mesh or a porous balloon, such that fluid can flow into and out of the spacing member 950. In some embodiments, the spacing member 950 can be formed of a non-porous balloon. In some embodiments, the spacing member 950 can be sufficiently non-porous that the spacing member 950 can prevent or obstruct fluid flow beyond the spacing member when the spacing member 950 is in the expanded configuration and in contact with walls defining the treatment region TR. In some embodiments, the spacing member 950 is sufficiently porous or defines sufficiently large openings such that at least some fluid can flow through the spacing member 950 (e.g., blood or CSF) while some flow and/or particles above a certain size are obstructed from flowing through or beyond the spacing member 950.

In some embodiments, the spacing member 950 can be coupled to the distal end of catheter body 920 and configured to be actuated (e.g., expanded from an initial configuration to the expanded configuration) by fluid delivered through the first fluid conduit 940. In some embodiments, spacing member 950 can be coupled to or integrally formed with another component of the catheter 910. For example, spacing member 950 can be formed with or coupled to an inner body (such as inner body 148 described, for example, with respect to FIG. 5A) that can function as a spacing member actuator 952 (e.g., a spacing member actuator that can be the same as or similar in structure and/or function to spacing member actuator 152) that is disposed within catheter body 920. The spacing member actuator 952 can be translated relative to the catheter body 920 (e.g., can be extended from the proximal end to the distal end of the catheter body 920 and urged distally) such that the spacing member 950 can be transitioned between a collapsed configuration and an expanded configuration (e.g., via being transitioned from being disposed within and constrained by the catheter body 920 and a region distal of the catheter body 920 such that the spacing member 950 can expand to the expanded configuration toward which the spacing member 950 may be biased).

In some embodiments, catheter 910 can also optionally include an imager 960 coupled to the distal end of catheter body 920. The imager 960 may be optically coupled to the image display ID by an imaging conduit 962 that may be disposed within the catheter body 920 and extend from the proximal end to the distal end of the catheter body 920.

Each component of the treatment system 900 can be implemented in various ways. For applications in which the catheter 910 is to be used to access the treatment region TR of a body lumen BL endovascularly, for example, the catheter 910 can be implemented as a conventional endovascular catheter, including its construction and materials, the ability to steer or not steer or deflect the distal end, to be deliverable over a guide wire or not, and include user controls and fittings at the proximal end. In some embodiments, the guide wire (not shown) can be disposed in the working channel 924 of the catheter body 920. In other embodiments, e.g., in which the catheter body 920 may be relatively large, the catheter body 920 may include a dedicated guide wire lumen, separate from the working channel 924. In some embodiments, the proximal portion of the catheter body 920 can be stiffer than the distal portion to provide sufficient rigidity for a user to push the catheter body 920 over the guide wire and through the lumen, e.g., vasculature. The more flexible distal portion can facilitate navigation of the catheter body 920 through, for example, tortuous vasculature. The catheter body 920 may be introduced into the body lumen BL, such as a blood vessel, via a cut down or other percutaneous technique for accessing the vessel lumen. In some applications, the catheter 910 may be used to access treatment region TR directly, rather than through the subject's vasculature, and may be implemented accordingly. For example, if the catheter 910 is used to access a treatment region TR directly through soft tissue, it may be implemented as a relatively rigid needle inserted through a trocar. In some embodiments, the catheter 910 can be used to access a body cavity BC, such as a CSF space (i.e., a space containing CSF) or a synovial joint space, via any of the access locations and methods described herein. For example, the catheter 910 can be used to access a body cavity BC surgically (e.g., via a burr hole or a craniectomy), via a minimally invasive approach, such as using a micropuncture kit at the cervical, thoracic, or lumbar spine level, or using a pre-existing access site.

As described above with respect to the light emitter 130, the light emitter 930 may be implemented with any known, suitable construction for emitting light of the desired wavelength and intensity from the distal end of the catheter 910 to the treatment region TR of the body lumen BL or body cavity BC. For example, in some embodiments, the light emitter 930 may simply be the end of an optical fiber, and the optical fiber may function as the light conduit 932 to convey light from the light source LS couplable to the proximal end of the catheter 910. The light source LS may be any suitable source of light of the desired wavelength and intensity, and may be a source of coherent light such as a laser (pulsed or continuous wave), or incoherent light (such as a xenon or halogen light and a suitable bandpass filter). In other embodiments, the light emitter 930 may be a relatively compact light source, e.g., a light emitting diode (LED) or a laser diode, disposed at the distal end of the catheter 910, to which electrical power is provided by a conductor extending from the proximal end of the catheter 910 through the catheter body 920 to the light source. In alternative embodiments, an LED or a laser diode can be disposed at the proximal end of catheter 910 (e.g., configured as the light source LS), and light can be conducted through a light conduit 932 and to the treatment region TR (e.g., reflected by a light emitter 930 disposed on the distal end of the light conduit 932).

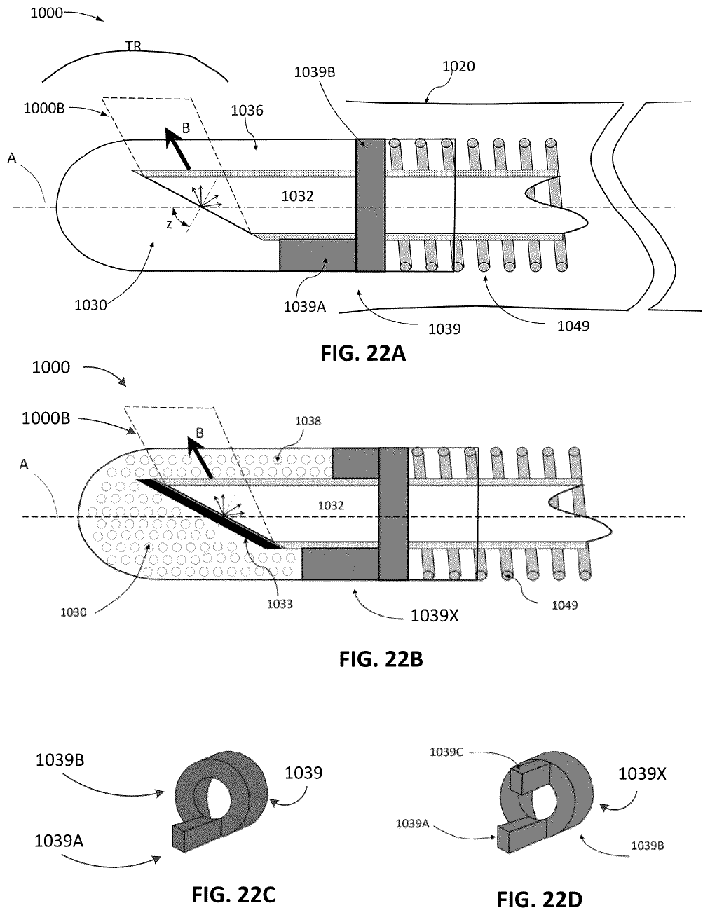

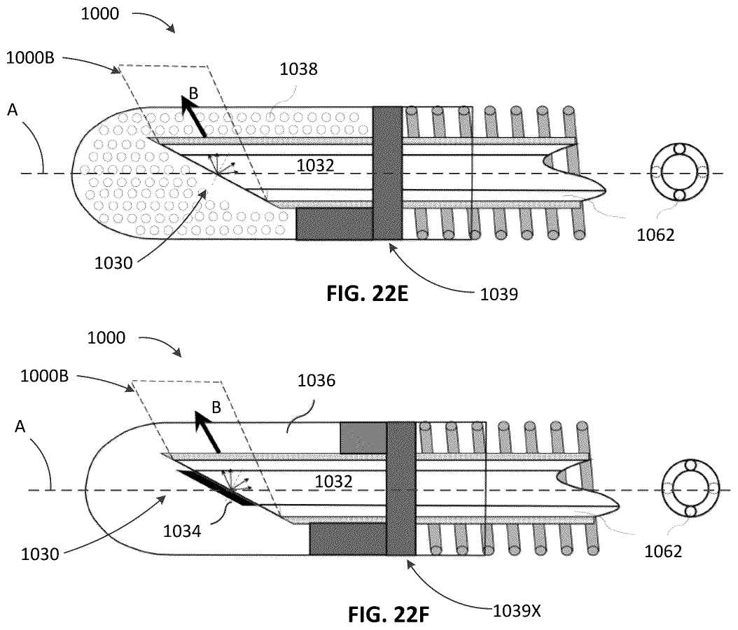

To produce a desired distribution of light at the treatment region TR (i.e., a distribution different from that produced by the light source LS and/or the light emitter 930 in combination), in some embodiments a light scatterer 936 is operatively associated with the light source LS to scatter light from the light source LS across the treatment region TR. The light scatterer 936 can be the same or similar in structure and/or function to any of the light scatterers described herein, such as the light scatterer 136. In some embodiments, for example, as shown schematically with respect to light scatterer 1036 in FIG. 22A, the light scatterer 936 may be implemented as a convex end cap on the distal tip of light conduit 932, which may be formed, for example, as an optical fiber. The end cap may include light scattering particles, illustrated, for example, as circular regions 1038 of light scatterer 1036 in FIG. 22B. Such particles may be, for example, titanium dioxide. Other light scattering materials (e.g., materials having a high refractive index of −2.5) or refracting structures may be used, such as, for example, diffraction gratings.

In use, the distal end of the catheter 910 can be disposed within a body cavity BC or body lumen BL of a subject near (e.g., adjacent) or within the treatment region TR (e.g., including a target tissue to be treated). The distal end of the catheter 910 can be disposed within the body cavity BC or the body lumen BL via any of the access locations, procedures, or devices (such as catheters) described herein, such as with respect to FIGS. 18A-20, and the body cavity BC or the body lumen BL may include any or the body cavities or body lumens described herein. The target tissue to be treated can be, for example, any of the target tissue described herein.

The spacing member 950 can be transitioned from the initial configuration to the expanded configuration. For example, the spacing member 950 can be transition to the expanded configuration such that the spacing member 950 stabilizes and maintains the light emitter 930 a particular distance (e.g., a predetermined distance) relative to the target tissue. The spacing member 950 can maintain the light emitter 930 centered within the body lumen BL or between opposing walls defining the body cavity BC.

Optionally, a cooling fluid can be provided via the catheter body 920 (e.g., via the first fluid conduit 940 or the working channel 924) to cool the light emitter 930. Optionally, a diluting fluid can be provided via the catheter body 920 (e.g., via the first fluid conduit 940 or the working channel 924) to dilute fluid within the body cavity BC or the body lumen BL to improve light transmission through the fluid to the target tissue. Optionally, a pressure (e.g., an equilibrium pressure) can be maintained within the body cavity BC or body lumen BL within a pressure range via at least one of providing fluid to the body cavity (e.g., via the first fluid conduit 940) or drawing fluid from the body cavity (e.g., via the second fluid conduit 941).

The light emitter 930 can be activated to emit light asymmetrically and at a non-zero angle relative to a central axis of the catheter body 920 onto the target tissue. The light emitter 930 can continue to emit the light until a therapeutic response or benefit is achieved. In some embodiments, the light emitter 930 may be or have a distal end that is configured to deliver the light in a lateral or sideways trajectory, rather than or in addition to axially relative to the light emitter 930 or the catheter body 920 or equally around a circumference of the light emitter 930. The light emitter 930 can illuminate a region of the interior wall that is on only one side of the light emitter 930 and/or the catheter body 920 (e.g., a minor arc portion relative to the light emitter 930), rather than illuminating a region around the entire circumference of the light emitter 930 and/or the catheter 910 or an area entirely distal and axially aligned with the light emitter 930. For example, the light emitter 930 can be configured to emit a laterally-directed beam to illuminate a region of the interior wall that has a length (e.g., an arc length) less than 5%, 10%, 15%, 20%, 30%, 40%, or 50% of the perimeter of a passageway or cavity within which the light emitter 930 is disposed taken within the same plane. Such asymmetrical and laterally emitted light distribution can better correlate to a shape of a treatment region TR or target tissue within the treatment region TR forming a body cavity BC (e.g., the surface of the brain).

In some embodiments, as described above, a system, such as any of the systems described herein, can be configured to emit light (e.g., therapeutic light) laterally and/or asymmetrically relative to a centerline of a catheter or light conduit such that the system can be used to apply targeted therapeutic light therapy to a treatment region and avoid a non-treatment region (e.g., a region adjacent to the treatment region or target tissue). In some embodiments, as described herein, a system, such as any of the systems described herein, can be configured to emit light having a first intensity (e.g., in the form of a light beam) laterally and/or asymmetrically to target a treatment region (e.g., a primary target treatment region), and to either emit light having a second intensity less than the first intensity to non-target treatment regions (e.g., secondary target treatment regions) or to not emit light to non-target treatment regions which may be adjacent to the target treatment region. For example, FIG. 22A is a schematic illustration of a portion of a system 1000. The system 1000 can be the same or similar in structure and/or function to any of the systems described herein, such as the system 900. As shown in FIG. 22A, the system 1000 includes a light conduit 1032 having a distal end forming a light emitter 1030 disposed within a light scatterer 1036 formed as a convex end cap. In some embodiments, the light scatterer 1036 can have a tubular portion and a dome-shaped distal end coupled to a distal end of the tubular portion. The light emitter 1030 can be disposed within the tubular portion and/or the dome-shaped distal end. The light conduit 1032 can be disposed within a working channel of a catheter body 1020, which can be the same or similar to any of the catheter bodies described herein. In some embodiments, the light conduit 1032 can be included in and/or the light emitter 1030 can be mounted on a distal end of an inner body, such as any of the inner bodies described herein (e.g., the inner body 148 described above). Thus, the light conduit 1032 and/or light emitter 1030 can be translatable relative to the catheter body (e.g., beyond a distal end of the catheter body of the system 1000). In some embodiments, the light scatterer 1036 can form an end cap of an inner body, the catheter body 1020, or a portion of the catheter body 1020. As shown in FIG. 22A, the light scatterer 1036 can be coupled to a flexible coil 1049 configured to provide stability to the distal end of the inner body or the catheter body 1020, particularly at the interface between the light scatterer 1036 and the catheter body 1020 or the inner body.

In some embodiments, rather than including a light scatterer 1036, the system 1000 can include a convex end cap having the same overall shape and size as the light scatterers described herein within which the light emitter 1030 can be disposed similarly as described herein relative to the light scatterers and through which light can be emitted from the light emitter 1030 to target tissue and/or non-target or secondary target tissue regions. For example, such a convex end cap can function to protect the light emitter 1030 (e.g., isolate the light emitter from a region distal of the light emitter) and can optionally have not light diffusion feature or function.

In some embodiments, as shown in FIG. 22A, the distal tip of the light conduit 1032 (e.g., the portion forming light emitter 1030) can be formed as an angled tip. For example, the distal tip of the light conduit 1032 can be cut at an angle relative to the central axis A of the light conduit 1032. The light conduit 1032 can be formed as a solid elongated member such that the angled cut at the distal end causes light traveling from a proximal end of the light conduit 1032 to reflect off of the angled distal end of the light conduit 1032 and travel laterally relative to the central axis A (e.g., at a non-zero angle relative to the central axis A) through a portion of a sidewall of the light conduit 1032 in the direction of arrow B, through the light scatterer 1036, and to the treatment region TR. In some embodiments, the angle Z (i.e., a light incident angle) between the normal to the distal surface of the light conduit 1032 and/or a light-contacting surface of the distal end of the light conduit 1032 and the central axis A (e.g., an acute angle as shown in FIG. 22A between the central axis A and the normal to the distal surface of the light conduit 1032) is sufficiently large (e.g., at or above a critical angle needed for total internal reflection) such that all of the light traveling through the light conduit 1032 will reflect from the distal surface and be emitted through the portion of sidewall of the light conduit 1032 facing the distal surface (i.e., facing a proximal side of the distal surface) (e.g., in the direction of arrow B). In some embodiments, the angle Z between the normal to the distal surface of the light conduit 1032 and/or a light-contacting surface of the distal end of the light conduit 1032 and the central axis A is small (e.g., below the critical angle needed for total internal reflection). When the angle Z is below the critical angle, a portion of the light traveling through the light conduit 1032 can be emitted distally of the light emitter 1030 (i.e., refracted along the central axis A and/or in a direction extending between the central axis A and the distal surface of the light conduit 1032) and a portion of the light traveling through the light conduit 1032 can be emitted laterally from the light emitter 1030 (e.g., reflected through the sidewall of the light conduit 1032). The light emitted laterally from the light emitter 1030 (e.g., a first portion of the light emitted from the light emitter and received via the light conduit 1032) can have a higher intensity than the light emitted distally of the light emitter (e.g., axially) (e.g., a second portion of the light emitted from the light emitter and received via the light conduit 1032). For example, FIG. 22E shows an embodiment of the system 1000 in which light can be dispersed both axially and laterally, with the light emitted in the direction of arrow B having a higher intensity than the light emitted axially along the central axis A and/or peripherally or laterally in directions other than the direction of arrow B. In some embodiments, both the first portion and the second portion of light emitted from the light emitter 1030 can travel through the light scatterer 1036, with the first portion directed through a first portion (e.g., a sidewall portion such as a sidewall of a tubular portion) of the light scatterer 1036 and the second portion directed through a second portion (e.g., a distal end portion such as a dome-shaped distal portion). In some embodiments, the angle of the distal surface of the light conduit 1032 and/or a light-contacting surface of the distal end of the light conduit 1032 can be formed at a critical angle or within a critical angle range such that rays of light passing through the light conduit 1032 and reaching the distal end of the light conduit 1032 are totally reflected and not refracted. For example, in some embodiments, the angle of the distal surface of the light conduit 1032 and/or a light-contacting surface of the distal end of the light conduit 1032 can be formed at a critical angle or within a critical angle range such that all rays of light passing through the light conduit 1032 and reaching the distal end of the light conduit 1032 are reflected along the distal surface. The critical angle can be calculated based on a numerical aperture of a fiber used as the optical fiber of the light conduit 1032 using the equation CA=sin⁻¹(NA), where CA represents the critical angle and NA represents the numerical aperture. The numerical aperture can be, for example, between 0.1 to 0.5. Thus, in some embodiments, the angle Z between the normal to the distal surface of the light conduit 1032 and the central axis A can range from about 5.74 degrees to about 30 degrees.

In some embodiments, as referenced above, the system 1000 can be configured to emit a beam of light to apply therapeutic light to a target tissue region and to simultaneously disperse light to a secondary tissue region. Such a system 1000 can thus deliver a combined effect of inhomogeneous light scattering that can be more diffused in a peripheral or outer region of a target therapy zone and relatively more focused or intense in a primary (e.g., central) portion of the target therapy zone. For example, the light emitter 1030 can emit a first portion of light emitted received via the light conduit 1032 laterally and/or asymmetrically (e.g., in the form of a beam) to be received at a primary target tissue region at a first intensity (e.g., such that the light received at the primary target tissue region has a first power density). The light emitter 1030 can simultaneously emit a second portion of the light received via the light conduit 1032 such that it is received by a secondary target tissue region at a second intensity less than the first intensity (e.g., such that the light received at the secondary target tissue region has a second power density less than the first power density). The second portion of the light emitted from the light emitter 1030 can, for example, travel distally from the light emitter 1030 to interact with (e.g., travel through) and be scattered by one or more light scatterers (e.g., the light scatterer 1036). In some embodiments, the second portion of the light scattered by the light scatterer 1036 can be dispersed spherically relative to the light scatterer(s) and/or the light emitter 1030 such that the light scatterer(s) and/or the light emitter 1030 are encircled by the secondary target tissue region that receives the diffused second portion of the light. In some embodiments, the secondary target tissue region can include portions of tissue that are distal of and/or proximal of the primary target tissue region, as well as portions of tissue that are on either side of the primary target tissue region between proximal and distal ends of the target tissue region (e.g., the secondary target tissue region can optionally surround the perimeter of the primary target tissue region). In some embodiments, the secondary target tissue region can include and/or surround the primary target tissue region such that the primary target tissue region also receives a portion of the second portion of the light. In some embodiments, for example, the system 1000 can include any suitable components described with respect to FIG. 3A and any suitable components described with respect to FIG. 22E such that light can be dispersed spherically by a diffuser (e.g., light scatterer 136 described with respect to FIG. 3A) and light can also be directed laterally (e.g., in the form of a beam) by the distal surface of the light conduit 1032 (as described with respect to FIG. 22E). In some embodiments, as shown in FIG. 22B, the light emitter 1030 can optionally include a reflective surface portion 1033 disposed at the distal end of the light conduit 1032 (e.g., disposed on the angled distal surface of the light conduit 1032). The reflective surface portion 1033 can be configured such that all light traveling to the distal end of the light conduit 1032 is directed through the sidewall of the light conduit 1032 in the direction of arrow B. The reflective surface portion 1033 can be configured to prevent or obstruct and light from traveling through the distal end of the light conduit 1032. The reflective surface portion 1033 can be, for example, a reflective prism surface. In such embodiments, the light conduit 1032 may be solid or may define a central lumen. Additionally, as shown in FIG. 22B, the light scatterer 1036 can optionally include particles (illustrated, for example, as circular regions 1038). Such particles may be, for example, titanium dioxide. Other light scattering materials (e.g., materials having a high refractive index of −2.5) or refracting structures may be used, such as, for example, diffraction gratings.

As shown in FIG. 22A, the system 1000 can optionally include a radiopaque marker 1039 coupled to the light emitter 1030 such that the user of the system 1000 can determine the location and the orientation of the light emitter 1030 prior to application of light from a light source through the light emitter 1030 (e.g., to apply radiation to the treatment region TR). As shown in FIG. 22C, which is a perspective view of the radiopaque marker 1039, the radiopaque marker 1039 can include a base portion 1039B (e.g., a circular base portion) and a projecting portion 1039A projecting from a non-central portion of the base portion 1039B. The base portion 1039B can define a central opening such that the light conduit 1032 can be disposed within the central opening of the base portion 1039B. The base portion 1039B can be coupled to the light emitter 1030 such that the projecting portion 1039A is stationary relative to the portion of the sidewall of the light conduit 1032 through which the light is directed from the distal end of the light conduit 1032 during operation of the system 1000. As shown, the projecting portion 1039A can be disposed on an opposite side of the light conduit 1032 from the portion of the sidewall through which emanating light (represented as box 1000B surrounding arrow B) is configured to be directed. Thus, when disposed within a patient, a user can visualize the location and orientation of the radiopaque marker 1039 using X-ray visualization and can determine the location and orientation of the portion of the sidewall through which light will be directed based on the location and orientation of the radiopaque marker 1039 (e.g., based on the orientation of the projecting portion 1039A relative to the base portion 1039B). If the user determines that the portion of the sidewall is not properly aligned with the treatment region TR based on the location and orientation of the radiopaque marker 1039, the user can manipulate the light emitter 1030 (e.g., via rotating and/or translating a portion of the system 1000 such as the light conduit 1032, an inner member, and/or a catheter body) relative to the treatment region TR. The radiopaque marker 1039 can be re-visualized and manipulated as needed until the portion of the sidewall of the light conduit 1032 is properly aligned with the treatment region TR such that light will be directed from the light emitter to the treatment region TR. Light can then be provided through the light conduit to the treatment region TR.

In some embodiments, as shown in FIG. 22B, the system 1000 can optionally include a radiopaque marker 1039X having two projecting portions. As shown in FIG. 22D, which is a perspective view of the radiopaque marker 1039X, the radiopaque marker 1039X can include the base portion 1039B and the projecting portion 1039A (also referred to as a first projecting portion 1039A) as shown and described above with respect to the radiopaque marker 1039 shown in FIG. 22C. Additionally, the radiopaque marker 1039X can include an additional projecting portion 1039C projecting from an opposite side of the base portion 1039B as the first projecting portion 1039A. Thus, the second projecting portion 1039C can be disposed on or adjacent a same portion or side of the light conduit 1032 through which emanating light (represented as box 1000B surrounding arrow B) is configured to be directed. As shown in FIG. 22D, the first projecting portion 1039A and the second projecting portion 1039C can have different lengths (e.g., the second projecting portion 1039C can be shorter than the first projecting portion 1039A). Thus, when disposed within a patient, a user can visualize the location and orientation of the radiopaque marker 1039X using X-ray visualization and can determine the location and orientation of the portion of the sidewall through which light will be directed based on the location and orientation of the radiopaque marker 1039X (e.g., based on the orientation of the first projecting portion 1039A relative to the second projecting portion 1039C and/or to the base portion 1039B). The inclusion of the second projecting portion 1039C can allow a user to more precisely orient the emanating beam of light from the light conduit 1032 compared to the radiopaque marker 1039. For example, the user can rotate the light conduit 1032 and radiopaque marker 1039 (which can be fixedly coupled) until the first projecting portion 1039A overlaps the second projecting portion 1039C (e.g., completely overlaps under X-ray visualization when viewing the target region TR from a location aligned orthogonally with a surface including the target region TR), which would indicate that the portion of the sidewall of the light conduit 1032 through which the light will be directed is aligned with or adjacent to the target region TR and that the system 1000 is oriented to direct light onto the target region TR (e.g., along a beam path in the direction of arrow B and represented as box 1000B in FIG. 22B). Although systems may be described herein and/or shown as including either radiopaque marker 1039 or radiopaque marker 1039X, any of the systems described herein can include one or the other.

As shown in FIG. 22E, which is a schematic illustration of a side view of a portion of the system 1000 and cross-sectional view of the light conduit 1032, in some embodiments, the light conduit 1032 can include a set of imaging conduits (e.g., optical fibers) 1062. For example, the light conduit 1032 can include or be coupled to four imaging conduits 1062. The imaging conduits 1062 can allow light and/or images from the treatment region TR to travel via the imaging conduits 1062 to an image display coupled to a proximal end of the light conduit 1032, such as the image display ID, such that a user can observe the treatment region TR using the light conduit 1032.

As shown in FIG. 22F, which is a schematic illustration of a side view of a portion of the system 1000 and cross-sectional view of the light conduit 1032, in some embodiments, the system 1000 can include an end cap 1034 coupled to the distal end of the light conduit 1032 and configured to aid in directing all light emitted from the light emitter 1030 radially relative to the central axis A of the light conduit 1032. In some embodiments, as shown in FIG. 22F, the end cap 1034 can have a sufficiently large diameter to cover at least an inner lumen defined by the light conduit 1032 while keeping the light path of imaging conduits 1062 unobstructed. In some embodiments, the end cap 1034 can be opaque. In some embodiments, the end cap 1034 can be configured to reflect light through the sidewall of the light conduit 1032 (e.g., in the direction of arrow B). In some embodiments, the end cap 1034 can include a reflective surface configured to reflect light through the sidewall (e.g., a reflective prism surface).

Although the system 1000 is shown as including the light conduit 1032, in some embodiments, the light emitter 1030 can include a light emitter, such as an LED or laser diode, disposed on the distal end of the catheter body 120 and configured to emit light that reflects off of a reflector such as the end cap 1034 or the reflective surface portion 1033 and travels laterally relative to a central axis of the light emitter and/or the catheter body 1020.

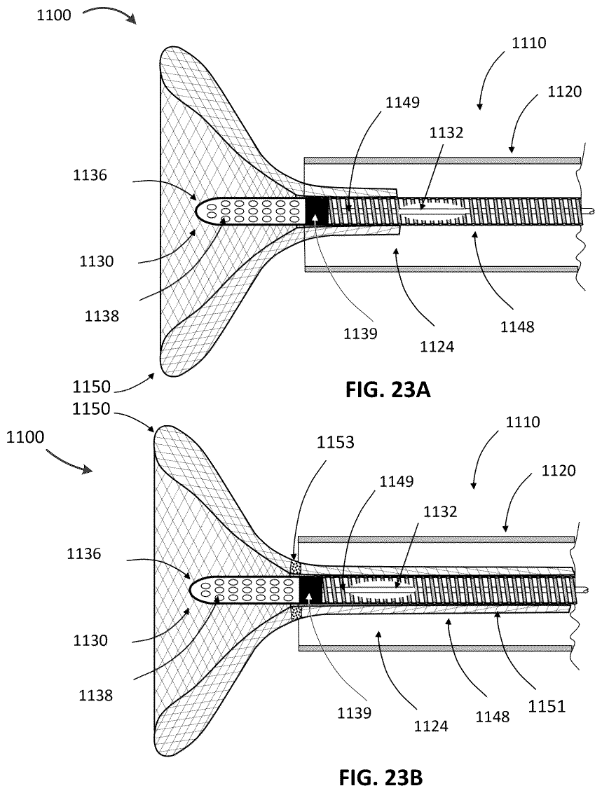

FIG. 23A is a schematic illustration of a cross-section of a portion of a system 1100. The system 1100 can be the same or similar in structure and/or function to any of the systems described herein. For example, the system 1100 can include a catheter 1110 including a catheter body 1120. The catheter body 1120 defines a working channel 1124. The system 1100 also includes an inner body 1148 within which a light conduit 1132 can be disposed. The light conduit 1132 can extend to or beyond a proximal end of the inner body 1148. A flexible coil 1149 can be included in or disposed within the inner body 1148, which can be the same or similar in structure and/or function to any of the coils described herein. The distal end of the inner body 1148 forms a light emitter 1130. A light scatterer 1136 can be disposed over the light emitter 1130. As shown, the light scatterer 1136 can optionally include light scattering portions 1138 (e.g., including light scattering particles). The system 1100 can optionally include a radiopaque marker 1139 coupled to the light emitter 1130 (e.g., disposed between the light emitter 1130 and a portion of the inner body 1148 proximal to the light emitter 1130) such that the user of the system 1100 can determine the location and/or the orientation of the light emitter 1130. The radiopaque marker 1139 can be any suitable shape or size (e.g., can have a tubular shape and can define one or more openings through which the light conduit 1132 and/or fluid can pass). In some embodiments, the radiopaque marker 1139 can be the same or similar in structure and/or function to the radiopaque marker 1039 described above.

The system 1100 includes a spacing member 1150 that can be the same or similar in structure and/or function to any of the spacing members described herein, such as the spacing member 150. The spacing member 1150 can be coupled to the inner body 1148 and/or the light scatterer 1136. For example, in some embodiments, the spacing member 1150 can include a neck portion coupled to an outer surface of the inner body 1148 along any suitable length of the inner body 1148 (e.g., a portion including the radiopaque marker and/or a portion proximal of the radiopaque marker and light emitter 1130 such as a portion having a length of about 0.5, 1, 2, 3, 4, 5, 6, 7, 8, 9, 10, 11, 12, 13, 14, 15, 16, 17, 18, 19, 20, 21, 22, 23, 24, or more centimeters proximal of the light emitter 1130). In some embodiments, the spacing member 1150 can be coupled to only a portion of the inner body 1148 containing the radiopaque marker. The spacing member 1150 can be configured to expand between an initial configuration and an expanded configuration (shown in FIG. 23A). In the expanded configuration, the spacing member 1150 can have a sufficient diameter or lateral extent such that the spacing member 1150 can contact oppositely disposed body structures (e.g., lumen or cavity walls). In some embodiments, the spacing member 1150 can be configured to maintain the light emitter 1130 a particular distance from the target tissue and/or centered between oppositely disposed body structures. Thus, the energy density of the light onto the target tissue (i.e., the energy per unit area of a cavity or vessel wall) can be controlled to be relatively uniform (i.e., to be maintained within a range of energy density values that is high enough to be therapeutically effective and low enough not to damage the cavity or vessel wall). In some embodiments, in the expanded configuration, as shown in FIG. 23A, the spacing member 1150 can be shaped such that the spacing member 1150 defines a pocket within which the light emitter 1130 can be at least partially disposed and/or from which the light emitter 1130 can project. In some embodiments, the spacing member 1150 can have a larger diameter at a distal end than at a proximal end in the expanded configuration. In some embodiments, the spacing member 1150 can have an outer surface and an inner surface that each taper from the larger diameter at the distal end to the smaller diameter at the proximal end in the expanded configuration. In some embodiments, the spacing member 1150 can be conically-shaped, cup-shaped, basket-shaped, plunger-shaped, or any other suitable shape in the expanded configuration.

For example, the spacing member 1150 can be biased toward the expanded configuration, but retained in the initial configuration within the working channel 1124 of the catheter body 1120. To transition the spacing member 1150 from the initial configuration to the expanded configuration, the inner body 1148 can be translated distally relative to the catheter body 1120 such that the spacing member 1150 is disposed distally of the distal end of the catheter body 1120 and is unconstrained such that the spacing member 1150 expands to the expanded configuration. In some embodiments, the spacing member 1150 can be configured, in the expanded configuration, to center the light emitter 1130 (e.g., the tip of an optical fiber forming the light conduit 1132) within a body lumen or body cavity. In some embodiments, the spacing member 1150 can be configured to expand to a shape and size within a body lumen or body cavity such that the spacing member 1150 contacts walls of the body lumen or body cavity to maintain the light emitter 1130 at a distance from the walls (e.g., centered within the body lumen or body cavity) but the spacing member 1150 does not urge the walls away from the light emitter 1130 such that a dimension (e.g., diameter) of the body lumen or body cavity is substantially enlarged or deformed by the spacing member 1150. Additionally or alternatively, in some embodiments, in the expanded configuration, the spacing member 1150 can be used to reduce blood flow beyond the spacing member 1150. Thus, the spacing member 1150 can be disposed relative to an aneurysm such that the spacing member 1150 reduces blood flow into the aneurysm to increase the residence time of cellular elements inside a treatment area such that the cellular elements receive a proper dose of irradiation. Additionally, reducing the blood flow beyond the spacing member 1150 can help to establish a translucent optical field within the treatment area. Furthermore, the spacing member 1150 can be used to temporarily prevent thrombi from escaping from an aneurysm during treatment.

In some embodiments, the spacing member 1150 can be configured to detach from the inner body 1148 and remain inside an aneurysm (e.g., permanently) to support the aneurysm after irradiation with the light emitter 1130. For example, the spacing member 1150 can serve as a neck supporting scaffold for progressive (e.g., reparative) tissue buildup inside the aneurysm.

The spacing member 1150 can be releasably coupled to the inner body 1148 via any suitable detachable coupling mechanism. For example, FIG. 23B is a schematic illustration of a cross-section of a portion of the system 1100 in which the spacing member 1150 includes or is couplable to an elongated neck portion 1151 via a sacrificial bond 1153 (also referred to herein as a sacrificial bonding layer 1153). The spacing member 1150 (or a distal expandable portion of the spacing member 1150) can be decoupled from the elongated neck portion 1151 at the location of the sacrificial bonding layer 1153. For example, the sacrificial bonding layer 1153 can be compromised by light emitted from the light emitter 1130. For example, at the end of a therapeutic phase of light delivery, the light intensity through the light emitter 1130 can be increased briefly to an intensity and for a duration sufficient to compromise the sacrificial bonding layer 1153 such that the spacing member 1150 (or a distal expandable portion of the spacing member distal of the sacrificial bonding layer 1153) decouples from the elongated neck portion 1151 and can be remain in the aneurysm when the remainder of the system 1100 is withdrawn relative to the aneurysm. As shown in FIG. 23B, the sacrificial bonding layer 1153 can be disposed in contact with and/or circumferentially surrounding a portion of the light emitter 1130 such that light from the light emitter can contact the sacrificial bonding layer 1153. Also as shown in FIG. 23B, the sacrificial bonding layer 1153 can optionally be disposed distally of some or all of the radiopaque marker 1139.

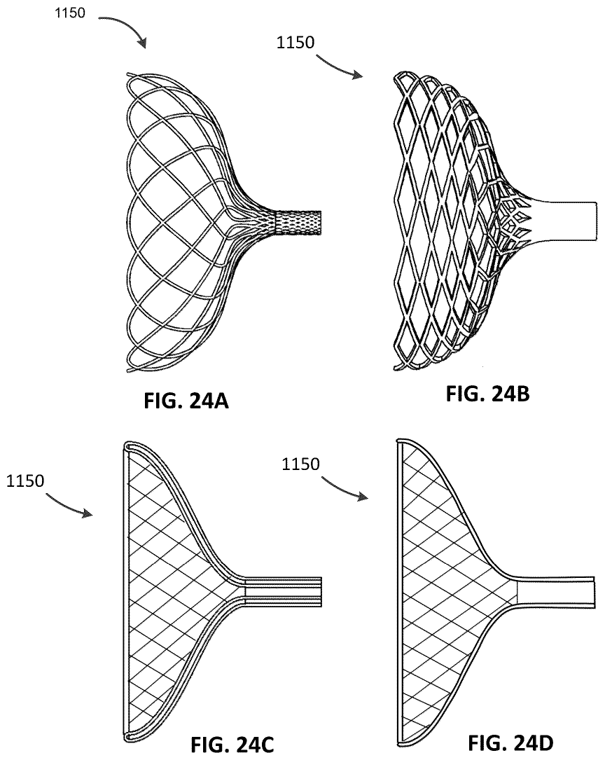

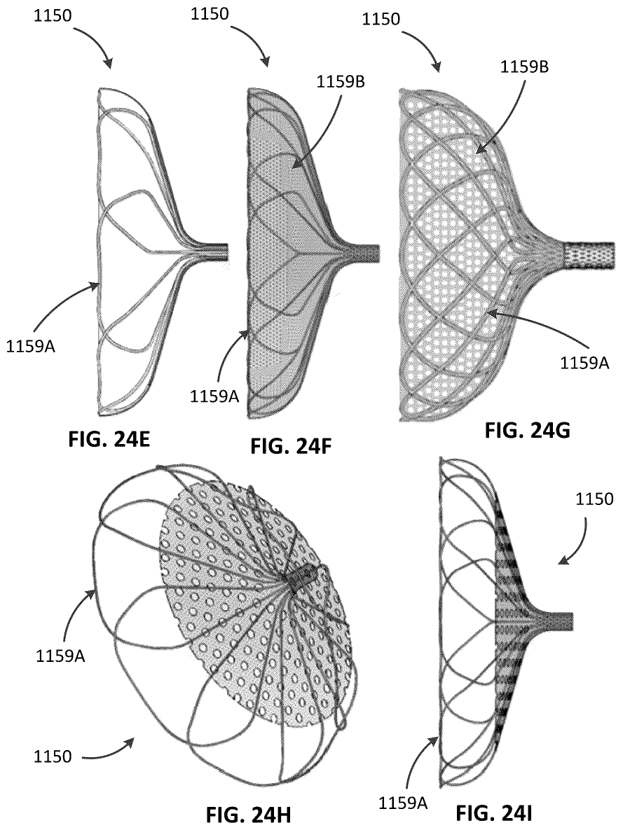

In some embodiments, the spacing member 1150 can include any suitable shape or structure, be made of any suitable material, and be formed using any suitable manufacturing method. For example, FIGS. 24A-24G are schematic illustrations of cross-sectional views of various spacing member 1150 embodiments. FIGS. 24H and 241 are a perspective view and a cross-sectional illustration of another version of the spacing member 1150, according to an embodiment. As shown in FIG. 24A, in some embodiments, the spacing member 1150 can be braided from a metal (e.g., Nitinol) or polymer wire. As shown in FIG. 24B, in some embodiments, the spacing member 1150 can be fabricated by laser cutting of a metal or a polymer sheet. As shown in FIG. 24C, in some embodiments, the spacing member 1150 can made of multiple layers. In some embodiments, the multiple layers can be distinct layers each having perimeters and/or ends distinct from those of other layers. In some embodiments, the multiple layers can be formed by one or more layers folded over themselves one or more times. Thus, for example, a distalmost end portion of the spacing member 1150 can be formed from a middle portion of a layer formed by a fold of the layer such that the distalmost end portion of the spacing member 1150 has a curved and/or continuous distal contact surface rather than including or being formed of a free end (e.g., a sharp or discontinuous end) of a layer or material. As shown in FIG. 24D, in some embodiments, the spacing member 1150 can made of a single layer of material.

In some embodiments, the spacing member 1150 can be fabricated as a composite of multiple elements. For example, the spacing member 1150 can include a first element (e.g., a frame) and second element (e.g., a cover) coupled to the frame and extending partially or completely from a proximal end to a distal end of the spacing member 1150. The first element can, for example, be fabricated by laser cutting of a metal or polymer sheet, be braided from metal (e.g., Nitinol) or a polymer wire, or be manually wound over a suitable jig in a braided or non-braided form. For example, FIGS. 24E and 24F each show a manually wound first element 1159A. As shown in FIG. 24F, for example, second element 1159B can be fabricated from a membranous polymer (e.g., PTFE) and be bonded to the first element 1159A. The surface of the membranous second element 1159B can be texturized to include mico-pores of known dimension, as shown in FIG. 24F, or can include macro-pores, as shown in FIG. 24G, with dimensions an order of magnitude or larger than the micro-pores providing texture to the surface to further accommodate hemodynamics and cellular proliferation on the surface. In some embodiments, the macro-pores can be sized the same as or similarly to the windows W described above with respect to FIG. 6. In some embodiments, as shown in FIGS. 24H and 241, which are a perspective view and cross-sectional side view of an embodiment of the spacing member 1150, the membranous second element 1159B can include both micro-pores and macro-pores. Alternatively, in some embodiments, the second element 1159B can be formed as an aggregation of polymer fibers such as an electro-spun mesh. The individual fibers can have a microtexture and macro holes/porosity can be provided by the density and arrangement of the fibers. The texturized membrane 1159B can span the entire surface of the spacing member 1150 or provide partial surface coverage (e.g., coverage of a first, proximal conical portion but not a second portion distal of the first portion) as shown in FIGS. 24H and 241. Thus, the membrane 1159B can extend at least partially from a proximal end to a distal end of the spacing member 1150.

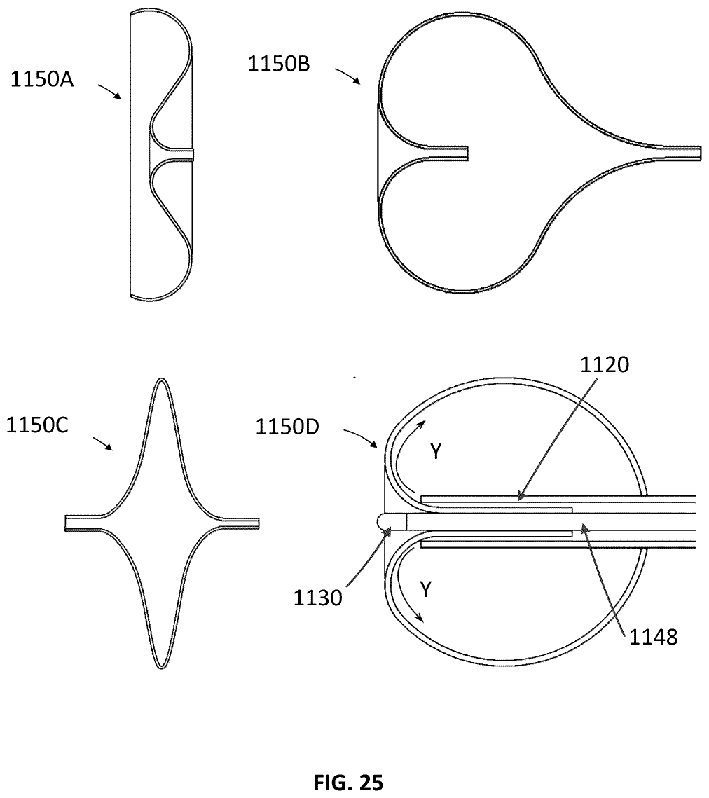

In some embodiments, the spacing member 1150 can have any suitable shape in the expanded configuration. For example, FIG. 25 includes schematic illustrations of cross-sectional views of various spacing member 1150 embodiments in an expanded configuration. As shown, each of the spacing members 1150 shown in FIG. 25 have different geometrical configurations. For example, the spacing member 1150A can assume in the expanded configuration (also referred to as the “open position”) a configuration that can reside at the neck of an aneurysm. As another example, the spacing member 1150B can assume the shape of a “pear” in the open position to expand and conform to the shape of the aneurysm after deployment. As another example, the spacing member 1150C can be formed of a continuous layer and can be configured to function as a neck bridging device having symmetry that allows for improved stability when deployed. As another example, the spacing member 1150D can assume a substantially spherical shape in the open position. The spacing member 1150D can be configured to deploy along the direction of the arrows Y such that the spacing member 1150D spreads out from a deployment point at a distal end of the catheter body 1120 and travels along a curved path proximally (e.g., back toward the proximal side of an aneurysm) toward a proximal portion of the catheter body 1120 (e.g., in a breaststroke fashion).

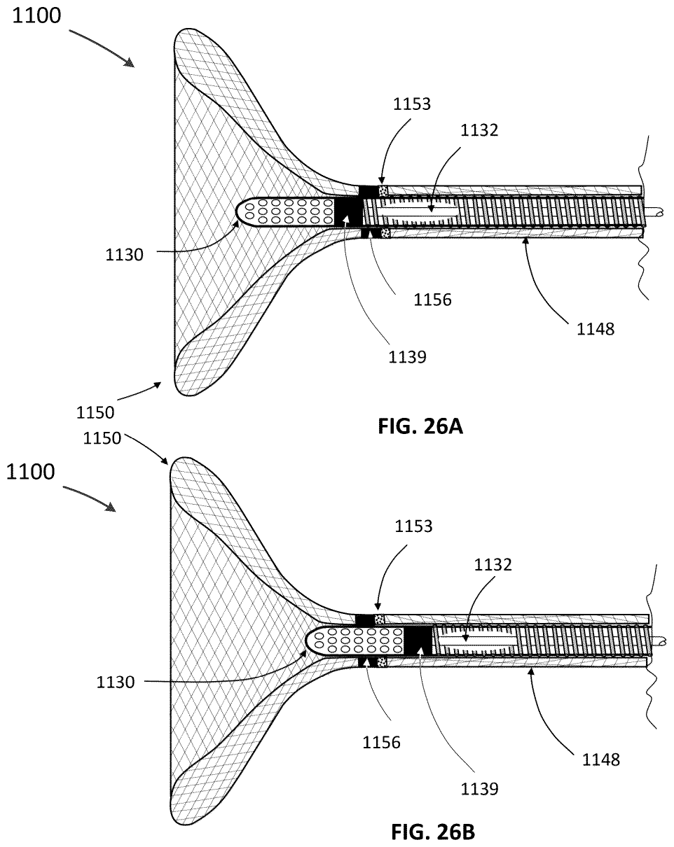

In some embodiments, the spacing member 1150 can be coupled to inner body 1148 and can be decouplable from the inner body 1148 via breaking up of a sacrificial bonding layer 1153. The light emitter 1130 can be translated relative to the sacrificial bonding layer 1153 to align the light emitter 1130 with the sacrificial bonding layer 1153 for breaking up of the sacrificial bonding layer 1153. For example, FIGS. 26A and 26B are schematic illustrations of a cross-section of a portion of the system 1100 in a first configuration and a second configuration, respectively. As shown, the spacing member 1150 is capable of being detached from the inner body 1148 by the use of a sacrificial bonding layer 1153. Compromising (e.g., breaking up) of the bonding layer 1153 is achieved by retracting the light emitter 1130 from a treatment position relative to the bonding layer 1153 (shown in FIG. 26A) to a detachment position relative to the bonding layer 1153 (shown in FIG. 26B). In the detachment position, a light source couple to the light emitter 1130 can be activated with the sufficient power to break up (e.g., dissolve) the sacrificial bonding layer 1153. In some embodiments, the detachment position (e.g., the position of the light emitter 1130 relative to the bonding layer 1153) can be confirmed prior to the activation of the detachment process by visualizing a change in location of the radiopaque marker 1139 and a radiopaque marker 1156 coupled to the spacing member 1150 and adjacent to the bonding layer 1153 relative to each other compared with their locations relative to each other in the treatment position.

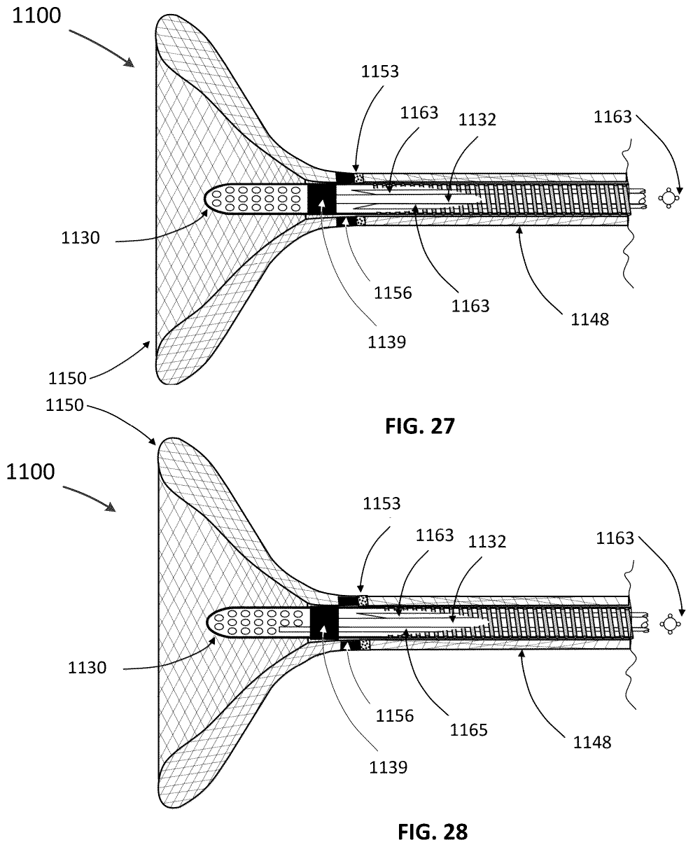

FIG. 27 is a schematic illustration of the system 1100 in which a plurality of optical fibers 1163 are embedded in the inner body 1148 and disposed around the light conduit 1132 (e.g., a central optical fiber) that delivers light to the light emitter 1130. The distal end of each of the optical fibers 1163 can all be polished at an angle smaller than the critical angle (e.g., the angle associated with total internal reflection) such that the light emanates from the distal end of the fibers in the direction of the sacrificial bond layer 1153. Additionally, the distal polished surface of the fibers 1163 can optionally be coated with a reflective layer (such as a mirror) to enhance the refraction of the light towards the sacrificial bond layer 1153. The sacrificial bond layer 1153 itself can be mixed with a light absorption substance to expedite the break up of the sacrificial bond layer 1153 when exposed to light from the fibers 1163. Once detached, the retraction of the inner body 1148 can be monitored by observing a change in the location of the radiopaque marker 1139 of the inner body 1148 relative to the location of the marker 1156 of the spacing member 1150. In some embodiments, the optical fibers 1163 can be used during the treatment phase to collect the scattered light that is imparted to the treatment area and transmit it as feedback to the user.

In some embodiments, rather than all of the optical fibers 1163 surrounding the light conduit 1132, one or more of the optical fibers 1163 can be elongated and can be at least partially embedded in the light emitter 1130. For example, FIG. 28 is a schematic illustration of the system 1100 in which an optical fiber 1165 of the plurality of optical fibers 1163 extends into the light emitter 1130 (e.g., beyond the radiopaque marker 1139). The optical fiber 1165 can be used as a light conduit that provides feedback on the amount of power that reaches the light emitter 1130 from the light source and is imparted to the treatment area. As before, the detachment of the spacing member 1150 can be monitored by the observing the change in the relative positioning of the radiopaque marker 1139 and the radiopaque marker 1156 relative to each other.

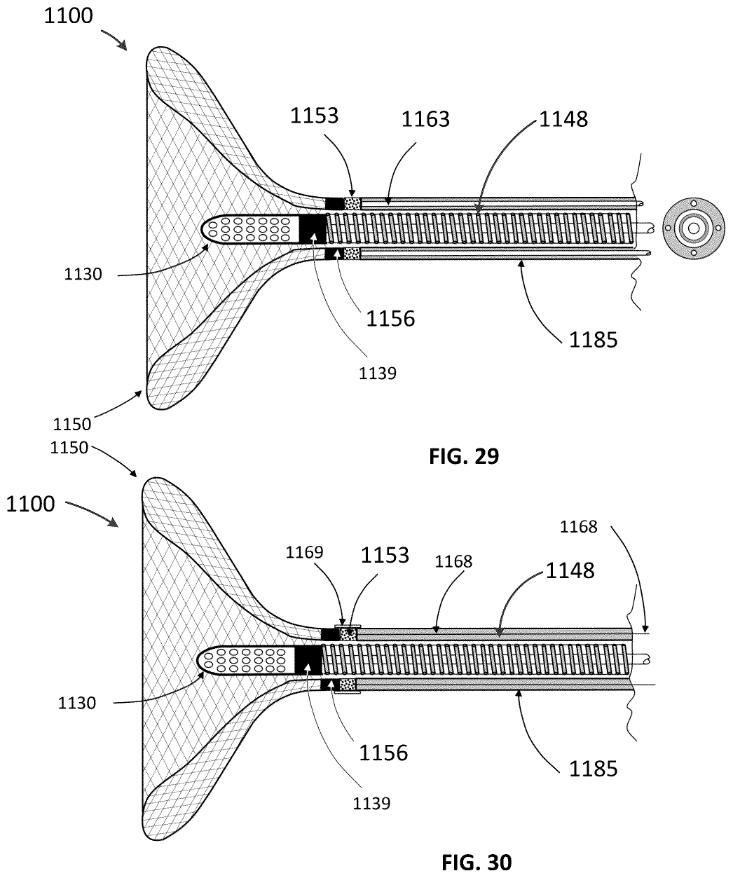

In some embodiments, rather than being detachably coupled to the inner body 1148, the spacing member 1150 can be detachably coupled to an outer body which can be an independent conduit within which the inner body 1148 can be disposed and translated. The outer body can be, for example, catheter body 1120 or another tubular body (e.g., a tubular body configured to be disposed within and translated relative to the catheter body 1020. For example, FIG. 29 is a schematic illustration of a cross-section of a portion of the system 1100 in which the spacing member 1150 is detachably coupled to the outer body 1185 via the sacrificial bond 1153. As shown in FIG. 29, the inner body 1148 is encased in the independent outer body 1185. As shown, the optical fibers 1163 that can transmit light energy to the sacrificial bond 1153 can be imbedded in the outer body 1185 instead of being located inside the inner body 1148. As shown, the distal ends of the optical fibers 1163 can be disposed adjacent to or in contact with the sacrificial bond 1153.

In some embodiments, the system 1100 can include electrical wires (e.g., within the catheter body 1120 or another independent outer conduit) to provide the energy needed to compromise the sacrificial bond 1153. For example, FIG. 30 is a schematic illustration of a cross-section of a portion of the system 1100 in which electrical wires 1168 are embedded inside the outer body 1185 to provide the energy needed to compromise the sacrificial bond 1153. As shown, insulation 1169 can be included to ensure that all the electrical energy passes through the sacrificial bond.

Another function realizable in this embodiment is a feedback system that can indicate the amount of energy that is provided by the light emitter 1130 to the tissue during the therapeutic phase by sensing the change in resistance of the spacing member 1150 and its connected electric leads 1168 in response to low level voltage across it. For example, the spacing member 1150 and its connected electrical leads can serve as a variable resistor (or one leg) of a Wheatstone bridge during exposure of the spacing member to light energy.

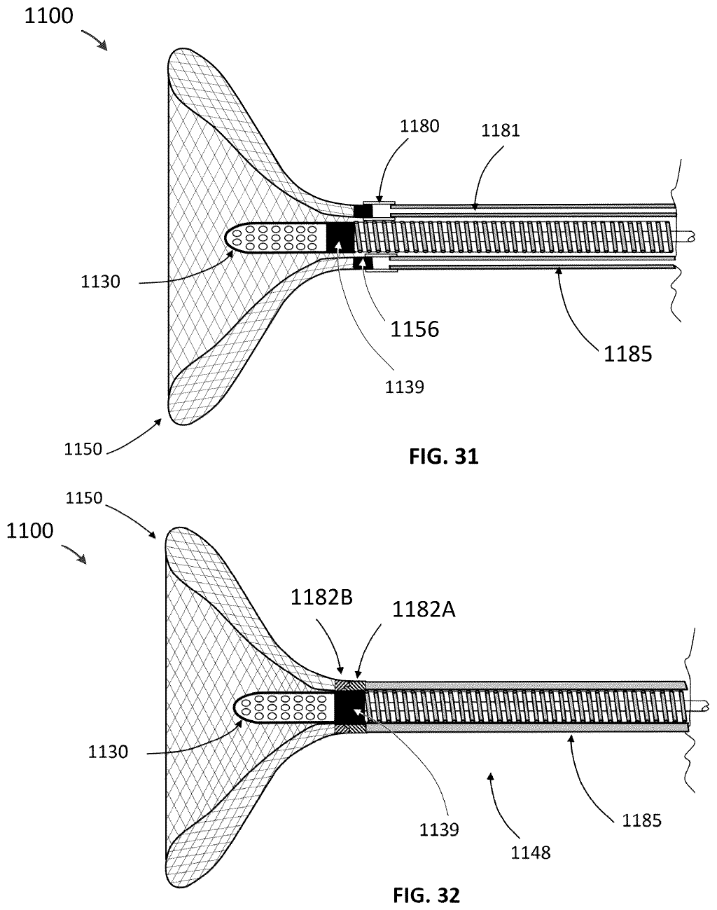

In some embodiments, the spacing member 1150 can be detachably coupled to the inner body and/or an outer body via a pressure rupturable hollow tube. For example, FIG. 31 is a schematic illustration of a cross-section of a portion of the system 1100 in which the spacing member 1150 (also referred to as a “centering mechanism” or a “centering member”) is detachably coupled to the inner body 1148 via a short length flexible hollow tube 1180. The hollow tube can be supplied with fluid through channels 1181 embedded in the outer conduit 1185. When detachment is desired, the fluid in the channels 1181 and the tube 1180 can be pressurized, causing the tube 1180 to expand radially and disconnecting the spacing member 1150 from the inner body 1148. In some embodiments, the tube 1180 can rupture to disconnect the spacing member 1150 from the outer conduit 1185.

FIG. 32 is a schematic illustration of a cross-section of a portion of the system 1100 including a separable friction fit between the spacing member 1150 (also referred to as a “centering mechanism” or a “centering member”) and the light emitter 1130 and/or the inner body 1148. For example, a neck portion of the spacing member 1150 can include a friction fit portion 1182B (e.g., a circumferential friction fit portion), the conduit 1185 can include a friction fit portion 1182A (e.g., a circumferential friction fit portion), and the light emitter 1130 and/or the marker 1139 and/or the inner body 1148 can include or function as a friction fit portion configured such that the external surface of the light emitter 1130 and/or the marker 1139 and/or the inner body 1148 and the internal surface of the spacing member 1150 and the conduit 1185 can be fit together tightly and held in place by friction (e.g., a neck portion of the spacing member 1150 can be coupled via a friction fit to the marker 1139 and the conduit 1185 can be coupled via a friction fit to the marker 1139). By pulling the inner body 1148 proximally while preventing the motion of independent conduit 1185 (e.g., holding conduit 1185 stationary), the friction fit between the light emitter 1130, the marker 1139, and/or the inner body 1148 and a neck portion of the centering member 1150 can be compromised. Thus, the inner body 1148 can be retracted and removed, the independent conduit 1185 can be retracted and removed relative to the centering member 1150, and the centering member 1150 can remain in the treatment region. In some implementations, the independent conduit 1185 can be held in place after the inner body 1148 is completely removed and can be used as a conduit for access to the treatment site within which the centering member 1150 is disposed for further treatment (e.g., delivery and/or aspiration of fluids and/or light treatment). Once the treatment is complete, the independent conduit 1185 can be removed.

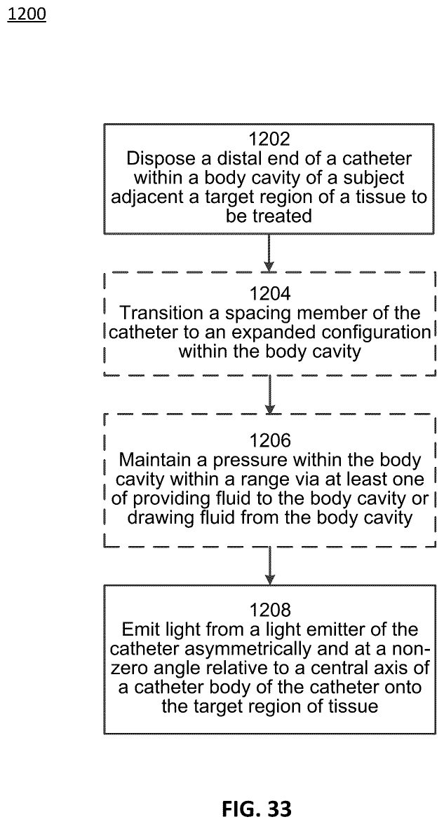

FIG. 33 is a flow chart illustrating a method 1200 of treating a treatment region of a body cavity. At 1202, a distal end of a catheter can be disposed within a body cavity of a subject near (e.g., adjacent) a target region of tissue to be treated. The catheter can include, for example, a catheter body and a light emitter. The light emitter can be configured to emit light asymmetrically and at a non-zero angle relative to a central axis of the catheter body. At 1204, optionally, an optional spacing member of the catheter can be transitioned to an expanded configuration within the body cavity. At 1206, optionally, a pressure (e.g., an equilibrium pressure) can be maintained within the body cavity (e.g., within a range) via at least one of providing fluid to the body cavity or drawing fluid form the body cavity. In some embodiments, the providing the fluid to the body cavity and the drawing fluid from the body cavity can be performed simultaneously. At 1208, light can be emitted from the light emitter asymmetrically and at a non-zero angle relative to a central axis of the catheter body onto the target region of tissue. In some embodiments, although the light is described as being emitted asymmetrically, the light can be emitted symmetrically relative to the central axis of the catheter body onto the target region of tissue. Optionally, in some embodiments, as described above, light can also be dispersed to a region outside of the target region of tissue at an intensity smaller than the intensity of the light emitted to the target region of tissue.

In some embodiments, the target region can include brain tissue, and the body cavity can be a space adjacent the brain tissue including cerebrospinal fluid. In some embodiments, the body cavity can be a synovial joint cavity. In some embodiment, the target region includes spinal disks. In some embodiments, the target region includes damaged brain tissue, such as any of the damaged brain tissue described herein. In some embodiments, the target region can include the thalamus, and the distal end of the catheter can be disposed adjacent the target region by translating the distal end of the catheter through a CSF space of a third ventricle of the subject. In some embodiments, the target region can include the spinal cord, and the distal end of the catheter can be disposed adjacent the target region by translating the distal end of the catheter through a CSF space at least one of anterior or posterior of the spinal cord. In some embodiments, the target region is at least one of the thalamus, the subthalamic nucleus, or the caudate nucleus, and the distal end of the catheter can be disposed adjacent the target region by translating the distal end of the catheter through a CSF space of the ventricular system. In some embodiments, the target region or body cavity can include a cSDH cavity.

In some embodiments, the catheter can include a first fluid conduit and a second fluid conduit, and the method 1200 can include maintaining a pressure within the body cavity within a range via at least one of providing fluid to the body cavity via the first fluid conduit or drawing fluid from the body cavity via the second fluid conduit. In some embodiments, the providing the fluid to the body cavity and the drawing fluid from the body cavity can be performed simultaneously.

In some embodiments, the catheter can include a light conduit at least partially disposed within the catheter body, the light emitter disposed at a distal end of the light conduit, and light can be configured to be provided to the light emitter via the light conduit. In some embodiments, the light emitter can be formed by a distal end portion of the light conduit, and the emitting light includes emitting light through a sidewall of the light conduit.

In some embodiments, the catheter can include a spacing member configured to transition between a collapsed configuration and an expanded configuration. In some embodiments, the spacing member can be configured to prevent the light emitter from contacting tissue walls defining the body cavity (e.g., the target region). The spacing member can be configured to maintain the light emitter approximately centered with respect to at least one axis of the spacing member in the expanded configuration. The method 1200 can include transitioning the spacing member from the collapsed configuration to the expanded configuration within the body cavity such that the light emitter is centered between opposing tissue walls defining the body cavity. In some embodiments, the spacing member is at least partially transmissive and/or transflective of the light emitted from the light emitter, and the emitting light includes emitting light through the spacing member.

In some embodiments, the method includes emitting light at a wavelength in the visible portion of the spectrum. In some embodiments, the method includes emitting light at a wavelength in the near infrared portion of the spectrum. In some embodiments, the emitting light includes emitting light at a power and for a duration sufficient to deliver to the target region of tissue an amount of light energy sufficient to recruit stem cells locally and/or remotely, initiate activation, differentiation, and proliferation of the cells including multipotent stem cells, blood forming stem cells, and/or mesenchymal stem cells, vascular stem cells, endothelial precursor or progenitor cells, neuronal and glial progenitor cells, neural stem cells, and/or differentiated cells such as fibroblasts and collagen to produce the photochemical effect to the target region of tissue. In some embodiments, the light is emitted at a wavelength between 400 nm and 1,100 nm. In some embodiments, the light is emitted at a wavelength, for example, of 532 nm. In some embodiments, the power of the light applied to the target region may be in the range from 1 mW to 500 mW. In some embodiments, the power can be a range of 100 mW to 200 mW. In some embodiments, the power density of the light applied to the target region may be in the range of 1 mW/cm² to 5 W/cm². In some embodiments, the power density can be in a range of 5 mW/cm² to 500 mW/cm². In some embodiments, the power density can be in a range of 50 mW/cm² to 500 mW/cm². In some embodiments, the power density can be in a range of 175 mW/cm² to 200 mW/cm². In some embodiments, the light can be emitted to the target region in a single irradiation or multiple irradiations in a single session, and in a single session or over multiple sessions. In some embodiments, the light emitted from the light emitter can be pulsed. In some embodiments, the energy dose (e.g., a total energy dose delivered in a single irradiation session or procedure or a total cumulative energy dose delivered over multiple sessions) to a target region can be between 0.05 J/cm² and 250 J/cm². In some embodiments, the emitting light includes not emitting light onto a non-target region of tissue adjacent to the target region of tissue. In some embodiments, the emitting light includes emitting light at a lower intensity (e.g., to result in a lower power density) onto a non-target region of tissue (e.g., a secondary region) adjacent to the target region of tissue.

In some embodiments, the catheter includes a radiopaque marker coupled to the light emitter, and the method 1200 includes visualizing the radiopaque marker within the body cavity and manipulating the catheter based on the visualization of the radiopaque marker to adjust at least one of the orientation or the location of the light emitter relative to the target region of tissue.

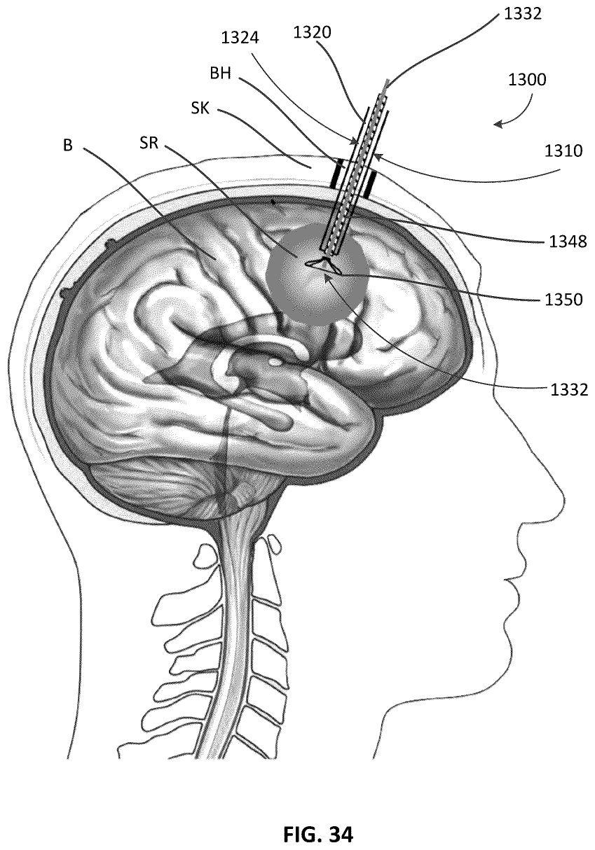

FIG. 34 is a schematic illustration of a treatment system 1300, which may be the same or similar as any of the treatment systems or devices described herein, being used to treat a stroke region SR of a brain B of a subject. The stroke region SR can include, for example, stroke damaged tissue. The embodiments and particular components of the treatment system 1300 shown and described with respect to FIG. 33 can be constructed the same as or similar to and include the same or similar features as corresponding components of any of the systems or devices described herein, such as the system 100, the system 900, and the system 1100 described above. The treatment system 1300 can be used to treat stroke damaged tissue with light energy using, for example, any of the methods described herein to achieve any of the intended effects or results described herein.

As shown in FIG. 34, the treatment system 1300 can include a catheter 1310 shown with a distal end portion of the catheter 1310 disposed within the stroke region SR. The catheter 1310 may include any of the features described above, for example with respect to catheters 110, 910, or 1110. In some embodiments, catheter 1310 can be a blunt needle. In some embodiments, catheter 1310 can be inserted over a trocar into the stroke region SR. As shown, a burr hole BH can be formed through a skull SK of the subject, and the catheter 1310 can be inserted through the burr hole BH to dispose a distal end of the catheter in or near the stroke region SR.

The treatment system 1300 can include a light emitter that can be the same or similar in structure and/or function to any of the light emitters described herein. In some embodiments, the treatment system 1300 can also include an inner body 1348. The inner body 1348 can be the same or similar in structure and/or function to any of the inner bodies described herein. For example, the inner body 1348 can include or be coupled to the light emitter. Additionally, the inner body 1348 can include and/or define a lumen configured to receive a light conduit 1332 (e.g., an optical fiber) coupled to the light emitter. The light emitter (and optional light conduit 1332) can be coupled to a light source (not shown).

The treatment system 1300 can include a spacing member 1350 that can be the same or similar in structure and/or function to any of the spacing members described herein. For example, the spacing member 1350 can be disposable at the distal end of a catheter body 1320 of the catheter 1310 (e.g., via a working channel 1324 of the catheter body 1320). The spacing member 1350 can be deployable to prevent the light emitter from directly contacting brain tissue of the brain B. For example, the spacing member 1350 can be deployable to maintain a minimum spacing between the light emitter of the treatment system 1300 and the walls of the treatment region defining the stroke region SR.

In treatment approach shown in FIG. 34, as described above, the catheter 1310 can be inserted into the stroke region SR through the burr hole BH. The spacing member 1350 can be deployed via any suitable method described herein. Light can then be emitted from the light emitter via any suitable method described herein to target stroke damaged brain tissue for treatment.

In some embodiments, the working channel 1324 of the catheter body 1320 (e.g., a first fluid conduit of the working channel 1324) can be used to introduce or inject fluid (e.g., saline) from a fluid source (not shown) into the treatment region, i.e., the stroke region SR. Since fluid in the stroke region SR of the brain B is typically transparent or translucent, no dilution of such fluid with a transparent fluid such as saline may be necessary in order to have the light irradiation reach the brain tissue to be treated. The fluid can be used to cool the stroke region SR (e.g., during the application of light from the light emitter). In some embodiments, if light scattering material that is fully transmissive, e.g., diamond dust, is used (e.g., a light scatterer such as any of the light scatterers described herein coupled to a distal end of the inner body 1348 and/or covering the light emitter), no cooling may be required. The catheter body 1320 can define or include a second fluid conduit to aspirate (e.g., remove) excess fluid and/or other material from the treatment region (e.g., continuously or intermittently, such as when during periods in which fluid is being introduced via the first fluid conduit). Additionally, fluid delivery through the first fluid conduit and aspiration through second fluid conduit can be controlled to avoid an increase in pressure (e.g., CSF pressure) in the treatment region (e.g., outside of an unsafe pressure range). For example, fluid can be delivered through the first fluid conduit and aspirated through the second fluid conduit at about the same flow rate such that the pressure in the treatment region remains unchanged or within a safe range during the procedure.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Where schematics and/or embodiments described above indicate certain components arranged in certain orientations or positions, the arrangement of components may be modified. While the embodiments have been particularly shown and described, it will be understood that various changes in form and details may be made. Any portion of the apparatus and/or methods described herein may be combined in any combination, except mutually exclusive combinations. The embodiments described herein can include various combinations and/or sub-combinations of the functions, components, and/or features of the different embodiments described.

1. A system, comprising:
a catheter body;
a light conduit at least partially disposed within the catheter body;
a light emitter disposed at a distal end of the light conduit and, when disposed in a body cavity of a patient near a target tissue region, configured to emit a first portion of light received through the light conduit asymmetrically and at a non-zero angle relative to a central axis of the light conduit such that the first portion is transmitted to the target tissue region at a first intensity and to emit a second portion of the light received through the light conduit distally of the light emitter; and
a light scatterer coupled to the light emitter and configured to diffuse the second portion of light such that the second portion of light is transmitted to a non-target tissue region at second intensity lower than the first intensity.

2. The system of claim 1, wherein the light emitter includes a distal end surface of the light conduit, the distal end surface disposed at a non-zero angle relative to a central axis of the light conduit such that the first portion of light transmitted through the light conduit to the distal end surface is reflected through a sidewall of the light conduit by the distal end surface and the second portion of light travels through the distal end surface.

3. (canceled)

4. The system of claim 1, wherein the light scatterer and the light emitter are configured such that the first portion is transmitted to the target tissue region through a sidewall of the light emitter.

5.-6. (canceled)

7. The system of claim 1, wherein the light scatterer and the light emitter are configured such that the second portion of light is diffused by the light scatterer axially and laterally relative to a central axis of the light scatterer, the light scatterer and the light conduit being coaxial.

8. (canceled)

9. The system of claim 1, further comprising a spacing member configured to transition between a collapsed configuration and an expanded configuration, in the expanded configuration the spacing member configured to be disposed about the light emitter to maintain the light emitter approximately centered with respect to at least one axis of the spacing member, the spacing member configured to maintain the light emitter centered between opposing tissue walls defining the body cavity in the expanded configuration.

10.-11. (canceled)

12. The system of claim 9, wherein the spacing member has a conical shape in the expanded configuration such that the spacing member defines a conical space within which the light emitter is at least partially disposed.

13. The system of claim 9, wherein the spacing member has an open distal end and defines an interior space in the expanded configuration within which the light emitter is at least partially disposed, the spacing member including a frame portion and a membranous cover portion.

14.-17. (canceled)

18. The system of claim 1, further comprising a light source coupleable to a proximal end of the light conduit and configured to generate light at a wavelength in the visible portion of the electromagnetic spectrum and such that the target tissue region receiving the light transmitted from the light source to the target tissue region via the light conduit and the light emitter is provided an energy dose of between 0.05 J/cm² and 250 J/cm² to the target tissue region.

19. (canceled)

20. A system, comprising:
a catheter body;
a light conduit at least partially disposed within the catheter body; and
a light emitter disposed at a distal end of the light conduit and configured to emit light asymmetrically and at a non-zero angle relative to a central axis of the light conduit such that the light is transmitted to a target tissue region when the light emitter is disposed in a body cavity of a patient near the target tissue region.

21. (canceled)

22. The system of claim 20, wherein the catheter body defines a first fluid conduit configured to be fluidically coupled to a fluid source and a second fluid conduit configured to be coupled to a fluid sink, the first fluid conduit and the second fluid conduit configured to be in fluid communication with the body cavity such that a pressure within the body cavity can be maintained within a range via at least one of providing fluid from the fluid source to the body cavity via the first fluid conduit or drawing fluid from the body cavity to the fluid sink via the second fluid conduit.

23. (canceled)

24. The system of claim 20, wherein the light conduit is formed as a solid elongated member and the light emitter includes a distal end surface of the light conduit, the distal end surface disposed at a non-zero angle relative to a central axis of the light conduit such that light transmitted through the light conduit to the distal end surface is reflected through a sidewall of the light conduit by the distal end surface.

25. (canceled)

26. The system of claim 20, wherein the light emitter includes a distal end surface of the light conduit and a reflective surface portion disposed on the distal end surface, the distal end surface and the reflective surface portion disposed at a non-zero angle relative to a central axis of the light conduit such that light transmitted through the light conduit to the distal end surface is reflected by the reflective surface portion through a sidewall of the light conduit.

27. (canceled)

28. The system of claim 20, further comprising a spacing member configured to transition between a collapsed configuration and an expanded configuration, in the expanded configuration the spacing member configured to be disposed about the light emitter to maintain the light emitter approximately centered with respect to at least one axis of the spacing member, the spacing member configured to maintain the light emitter centered between opposing tissue walls defining the body cavity in the expanded configuration.

29.-30. (canceled)

31. The system of claim 28, wherein the spacing member has a conical shape in the expanded configuration such that the spacing member defines a conical space within which the light emitter is at least partially disposed.

32.-36. (canceled)

37. The system of claim 20, further comprising a light source coupleable to a proximal end of the light conduit and configured to generate light at a wavelength in the visible portion of the electromagnetic spectrum and such that the target tissue region receiving the light transmitted from the light source to the target tissue region via the light conduit and the light emitter is provided an energy dose of between 0.05 J/cm² and 250 J/cm² to the target tissue region.

38. (canceled)

39. A system, comprising:
a catheter body defining a working channel;
an inner body translatable within the working channel and including a light emitter on a distal end of the inner body; and
a spacing member coupled to the inner body proximal of the light emitter, the spacing member configured to transition between a collapsed configuration and an expanded configuration, the spacing member having a conical shape in the expanded configuration such that the spacing member defines a conical space within which the light emitter is at least partially disposed, the spacing member configured to maintain the light emitter approximately centered with respect to a central axis of the spacing member when the spacing member is in the expanded configuration within a body lumen or between opposing walls of a body cavity.

40. The system of claim 39, wherein the spacing member is biased toward the expanded configuration, the spacing member configured to be retained in the collapsed configuration by the catheter body when disposed within the working channel of the catheter body and to expand upon the inner body being translated distally relative to the catheter body such that the spacing member is disposed distally of the catheter body.

41. The system of claim 39, wherein the spacing member is configured to prevent fluid from flowing from a region distal of the spacing member to a region proximal of the spacing member in the expanded configuration.

42. The system of claim 39, wherein the spacing member is configured to prevent objects above a threshold size from flowing from a region distal of the spacing member to a region proximal of the spacing member in the expanded configuration, while allowing fluid to flow from the region distal of the spacing member to the region proximal of the spacing member.

43. The system of claim 39, wherein the spacing member has a conical shape in the expanded configuration such that the spacing member defines a conical space within which the light emitter is at least partially disposed.

44.-45. (canceled)

46. A method, comprising:
disposing a distal end of a catheter within a body cavity of a subject near a target region of tissue to be treated, the catheter including:
a catheter body, and
a light emitter configured to emit light asymmetrically and at a non-zero angle relative to a central axis of the catheter body; and
emitting light from the light emitter asymmetrically and at a non-zero angle relative to the central axis of the catheter body onto the target region of tissue.

47. The method of claim 46, wherein the target region of tissue includes brain tissue, and the body cavity is a space adjacent the brain tissue including cerebrospinal fluid.

48. (canceled)

49. The method of claim 46, wherein the catheter includes a first fluid conduit and a second fluid conduit, the method further comprising:
maintaining a pressure within the body cavity within a range via at least one of providing fluid to the body cavity via the first fluid conduit or drawing fluid from the body cavity via the second fluid conduit.

50.-51. (canceled)

52. The method of claim 46, wherein the catheter includes a spacing member configured to transition between a collapsed configuration and an expanded configuration, in the expanded configuration the spacing member configured to maintain the light emitter approximately centered with respect to at least one axis of the spacing member, and further comprising:
transitioning the spacing member from the collapsed configuration to the expanded configuration within the body cavity such that the light emitter is centered between opposing tissue walls defining the body cavity.

53. The method of claim 52, wherein the spacing member is at least partially transmissive and/or transflective of the light emitted from the light emitter, and the emitting light includes emitting light through the spacing member.

54. (canceled)

55. The method of claim 46, wherein the emitting light includes emitting light at a power and for a duration sufficient to deliver to the target region of tissue an amount of light energy sufficient to recruit stem cells locally and/or remotely, and to initiate differentiation, activation and proliferation of the cells including multipotent stem cells, blood forming stem cells, and/or mesenchymal stem cells, vascular stem cells, endothelial precursor or progenitor cells, and/or differentiated cells such as fibroblasts and collagen to produce a photochemical effect in the target region of tissue.

56. The method of claim 46, wherein the emitting light includes emitting light at a wavelength between 400 nm and 1,100 nm.

57.-58. (canceled)

59. The method of claim 46, wherein the emitting light includes emitting light to provide an energy dose of between 0.05 J/cm² and 250 J/cm² to the target region of tissue.

60. The method of claim 46, wherein the emitting light includes not emitting light onto a non-target region of tissue adjacent to the target region of tissue.

61. (canceled)

62. The method of claim 46, wherein the target region of tissue includes at least a portion of a chronic subdural hematoma cavity.

63. (canceled)
